SEQUENCE LISTING (23 sequences)

SEQ ID NO: 1 - protein, 20 aa, synthetic construct
  REGION 1..20: note = Description of Artificial Sequence: Syntheticpeptide
  MOD_RES 10: note = D-amino acid
VKVKVKVKVPPTKVKVKVKV

SEQ ID NO: 2 - protein, 20 aa, synthetic construct
  REGION 1..20: note = Description of Artificial Sequence: Syntheticpeptide
  MOD_RES 10: note = D-amino acid
VKVRVRVRVPPTRVRVRVKV

SEQ ID NO: 3 - protein, 26 aa, synthetic construct
  REGION 1..26: note = Description of Artificial Sequence: Syntheticpeptide
  MOD_RES 10: note = D-amino acid
VKVRVRVRVPPTRVRVRVKVGRGDSP

SEQ ID NO: 4 - protein, 20 aa, synthetic construct
  REGION 1..20: note = Description of Artificial Sequence: Syntheticpeptide
  MOD_RES 10: note = D-amino acid
VRVRVKVRVPPTRVEVRVRV

SEQ ID NO: 5 - protein, 20 aa, synthetic construct
  REGION 1..20: note = Description of Artificial Sequence: Syntheticpeptide
  MOD_RES 10: note = D-amino acid
VRVRVRVKVPPTKVRVRVRV

SEQ ID NO: 6 - protein, 24 aa, synthetic construct
  REGION 1..24: note = Description of Artificial Sequence: Syntheticpeptide
  MOD_RES 14: note = D-amino acid
RGDSVRVRVRVKVPPTKVRVRVRV

SEQ ID NO: 7 - protein, 36 aa, synthetic construct
  REGION 1..36: note = Description of Artificial Sequence: Syntheticpolypeptide
  MOD_RES 10: note = D-amino acid
VKVRVRVRVPPTRVRVRVKVGKLTWQELYQLKYKGI

SEQ ID NO: 8 - protein, 31 aa, synthetic construct
  REGION 1..31: note = Description of Artificial Sequence: Syntheticpolypeptide
  MOD_RES 10: note = D-amino acid
VKVRVRVRVPPTRVRVRVKVRPKPQQFFGLM

SEQ ID NO: 9 - protein, 17 aa, synthetic construct
  REGION 1..17: note = Description of Artificial Sequence: Syntheticpeptide
VKVRVRVRVRVRVRVKV

SEQ ID NO: 10 - protein, 20 aa, synthetic construct
  REGION 1..20: note = Description of Artificial Sequence: Syntheticpeptide
  MOD_RES 10: note = D-amino acid
VKVKVRVKVPPTKVKVRVKV

SEQ ID NO: 11 - protein, 20 aa, synthetic construct
  REGION 1..20: note = Description of Artificial Sequence: Syntheticpeptide
VKVRVRVRYNGTRVRVRVKV

SEQ ID NO: 12 - protein, 28 aa, synthetic construct
  REGION 1..28: note = Description of Artificial Sequence: Syntheticpeptide
  MOD_RES 18: note = D-amino acid
LKKTETQGVKVRVRVRVPPTRVRVRVKV

SEQ ID NO: 13 - protein, 25 aa, synthetic construct
  REGION 1..25: note = Description of Artificial Sequence: Syntheticpeptide
  MOD_RES 15: note = D-amino acid
SDKPGVLVRVRVRVPPTRVRVRVKV

SEQ ID NO: 14 - protein, 24 aa, synthetic construct
  REGION 1..24: note = Description of Artificial Sequence: Syntheticpeptide
  MOD_RES 14: note = D-amino acid
RGDSVRVRVRVKVPPTKVRVRVRV

SEQ ID NO: 15 - protein, 20 aa, synthetic construct
  REGION 1..20: note = Description of Artificial Sequence: Syntheticpeptide
  MOD_RES 10: note = D-amino acid
VKVKVKVKVPPTKVEVKVKV

SEQ ID NO: 16 - protein, 20 aa, synthetic construct
  REGION 1..20: note = Description of Artificial Sequence: Syntheticpeptide
  MOD_RES 10: note = D-amino acid
VLTKVKTKVPPTKVEVKVLV

SEQ ID NO: 17 - protein, 28 aa, synthetic construct
  REGION 1..28: note = Description of Artificial Sequence: Syntheticpeptide
  MOD_RES 10: note = D-amino acid
VLTKVKTKVPPTKVEVKVLVGGGGRGDV

SEQ ID NO: 18 - protein, 24 aa, synthetic construct
  REGION 1..24: note = Description of Artificial Sequence: Syntheticpeptide
  MOD_RES 14: note = D-amino acid
RGDSVKVRVRVRVPPTRVRVRVKV

SEQ ID NO: 19 - protein, 26 aa, synthetic construct
  REGION 1..26: note = Description of Artificial Sequence: Syntheticpeptide
  MOD_RES 16: note = D-amino acid
IKVAVGVKVRVRVRVPPTRVRVRVKV

SEQ ID NO: 20 - protein, 26 aa, synthetic construct
  REGION 1..26: note = Description of Artificial Sequence: Syntheticpeptide
  MOD_RES 16: note = D-amino acid
YIGSRGVKVRVRVRVPPTRVRVRVKN

SEQ ID NO: 21 - protein, 18 aa, synthetic construct
  REGION 1..18: note = Description of Artificial Sequence: Syntheticpeptide
  MOD_RES 9: note = D-amino acid
VLTKVKTVPPTKVEVKVL

SEQ ID NO: 22 - protein, 28 aa, synthetic construct
  REGION 1..28: note = Description of Artificial Sequence: Syntheticpeptide
  MOD_RES 10: note = D-amino acid
VEVSVSVEVPPTEVSVEVEVGGGGRGDV

SEQ ID NO: 23 - protein, 20 aa, synthetic construct
  REGION 1..20: note = Description of Artificial Sequence: Syntheticpeptide
  MOD_RES 10: note = D-amino acid
VEVSVSVEVPPTEVSVEVEV

(19) United States
(12) Patent Application Publication
(10) Pub. No.: US 2025/0009931 A1
(43) Pub. Date: Jan. 9, 2025

(54) FILMS FORMED FROM SELF-ASSEMBLING PEPTIDE HYDROGELS

(71) Applicant: GEL4MED, INC., Brighton, MA (US)

(72) Inventors: Manav Mehta, Newton, MA (US); Seyedeh Zahra Moafi Madani, Burlington, MA (US); Hung-Yi Liu, Lowell, MA (US)

(21) Appl. No.: 18/682,498

(22) PCT Filed: Aug. 9, 2022
(86) PCT No.: PCT/US2022/074719

§ 371 Date: Feb. 9, 2024

Related U.S. Application Data
(60) Provisional application No. 63/231,160, filed on Aug. 9, 2021.

(51) Int. Cl. A61L 26/00; C07K 7/08; C07K 14/00

(52) U.S. Cl. CPC A61L 26/0028; A61L 26/0019; A61L 26/008; A61L 26/0085; C07K 7/08; C07K 14/001

(57) ABSTRACT

Preparations capable of forming films are disclosed. The preparations include a biocompatible polymer and a purified amphiphilic peptide including a folding group having a plurality of charged amino acid residues and hydrophobic amino acid residues arranged in a substantially alternating pattern and a turn sequence, and at least one functional group available for crosslinking. The purified amphiphilic peptide is crosslinked with the biocompatible polymer to form the film. Kits for producing the film are also disclosed. Methods of producing the film are also disclosed.

### CROSS-REFERENCE TO RELATED APPLICATIONS

This application claims priority under 35 U.S.C. § 119(e) to U.S. Provisional Application Ser. No. 63/231,160, titled “FILMS FORMED FROM SELF-ASSEMBLING PEPTIDE HYDROGELS,” filed on Aug. 9, 2021, which is incorporated herein by reference in its entirety for all purposes.

### STATEMENT REGARDING FEDERALLY SPONSORED RESEARCH

This invention was made with government support under Grant No. 1843682, awarded by the National Science Foundation (NSF). The government has certain rights in the invention.

### SEQUENCE LISTING

The instant application contains a Sequence Listing which has been submitted electronically in XML (eXtensible Markup Language) and is hereby incorporated by reference in its entirety. Said XML copy, created on Aug. 9, 2022, is named G2093-7007WO_SL.xml.

### FIELD OF TECHNOLOGY

Aspects and embodiments disclosed herein are directed toward systems and methods for the administration of self-assembling peptides.

### BACKGROUND

Tissue engineering involves the use of materials with suitable biochemical and physiological properties to replace, repair, and/or enhance biological tissues. The particular tissue involved may have certain mechanical and structural requirements for proper functioning. There is a need for materials which are easily tunable for use with a particular target tissue and have suitable biochemical and physiological properties for tissue engineering.

### SUMMARY

In accordance with one aspect, there is provided a preparation. The preparation may comprise a biocompatible polymer having at least two functional groups capable of undergoing covalent crosslinking with a peptide group. The preparation may comprise a purified amphiphilic peptide comprising a folding group having a plurality of charged amino acid residues and hydrophobic amino acid residues arranged in a substantially alternating pattern and a turn sequence, and at least one functional group available for covalent crosslinking. The purified amphiphilic peptide may be capable of undergoing covalent crosslinking with the biocompatible polymer by a chemical crosslinker molecule or a coupling chemical agent to form a film. The film may be porous.

In accordance with another aspect, there is provided a preparation. The preparation may comprise a biocompatible polymer capable of undergoing ionic or physical crosslinking with a peptide group. The preparation may comprise a purified amphiphilic peptide comprising a folding group having a plurality of charged amino acid residues and hydrophobic amino acid residues arranged in a substantially alternating pattern and a turn sequence, and at least one functional group available for crosslinking. The purified amphiphilic peptide may be capable of undergoing ionic or physical crosslinking with the biocompatible polymer to form a film. The film may be porous.

In some embodiments, the preparation may further comprise a buffer comprising an effective amount of an ionic salt to induce the purified amphiphilic peptide to form a predetermined secondary structure. The purified amphiphilic peptide and the buffer may be in the form of the predetermined secondary structure.

The preparation may be formulated as a film comprising a hydrogel in the form of at least one of a cryogel, a dehydrated hydrogel, and a hydrated hydrogel.

The preparation may comprise between about 0.15% by weight to about 10% by weight of the purified amphiphilic peptide.

In some embodiments, the purified amphiphilic peptide may have between about 10-200 amino acid residues.

In some embodiments, the folding group may have between about 10-50 amino acid residues.

The hydrophobic amino acid residues may be independently selected from glycine, alanine, valine, leucine, isoleucine, proline, phenylalanine, methionine, threonine, tryptophan, and combinations thereof.

In some embodiments, the hydrophobic amino acid residue may be valine.

The charged amino acid residues may be independently selected from arginine, lysine, histidine, and combinations thereof.

The folding group may have between 2 and 10 positively charged amino acid residues.

The folding group may have 6 positively charged amino acid residues selected from arginine and lysine.

In some embodiments, the charged amino acid residues may be negatively charged amino acid residues.

In some embodiments, the charged amino acid residues may be independently selected from aspartic acid, glutamic acid, and combinations thereof.

In some embodiments, the at least one functional group of the purified amphiphilic peptide comprises an amine, carboxyl, thiol, succinimidyl ester, maleimide, alkene, transcyclooctene, alkyne, ring-strained alkyne, dibenzylcyclooctyne, tetrazine, azide, or combinations thereof.

In some embodiments, at least one of the N-terminus and the C-terminus of the purified amphiphilic peptide is modified.

In some embodiments, the modification is an amidation.

In some embodiments, the amidation is a cysteine moiety.

In some embodiments, at least one of the N-terminus and the C-terminus of the purified amphiphilic peptide is free.

In some embodiments, the folding group has a sequence comprising Y[AY]_{N}[T][YA]_{M}Y, where A is 1-3 amino acids selected from one or more of basic, neutral, aliphatic, aromatic, polar, and charged amino acids, Y is 1-3 hydrophobic amino acids, T is 2-8 turn sequence amino acids, and N and M are each independently between 2 and 10.

In some embodiments, the folding group has a sequence comprising Y[XY]_{N}[T][YX]_{M}Y, where X is 1-3 charged amino acids, Y is 1-3 hydrophobic amino acids, T is 2-8 turn sequence amino acids, and N and M are each independently between 2 and 10.

In some embodiments, the turn sequence has 2-8 amino acid residues independently selected from a D-proline, an L-proline, aspartic acid, threonine, and asparagine.

In some embodiments, the turn sequence has 1-4 proline residues.

In some embodiments, the folding group has a sequence comprising (Z)c(Y)b(X)a-[(d)PP, (d)PG, or NG]-(X)a(Y)b(Z)c, where the turn sequence is (d)PP, (d)PG, or NG, (d)P is a D-proline, X is a charged amino acid, Y is a hydrophobic amino acid, Z is a hydrophobic amino acid or a polar amino acid, and a, b, and c are each independently an integer from 1-10.

In some embodiments, the peptide comprises an effective amount of charge balancing counterions.

In some embodiments, the counterions comprise at least one of acetate, citrate, and chloride counterions.

In some embodiments, the counterions comprise acetate counterions.

In some embodiments, the purified amphiphilic peptide is substantially free of chloride counterions.

In some embodiments, the purified amphiphilic peptide is at least 80% purified, for example, at least 85%, at least 90%, at least 92%, at least 95%, at least 98%, at least 99%, or at least 99.9%.

In some embodiments, the purified amphiphilic peptide has less than 10% residual organic solvent by weight, for example, less than 8%, less than 5%, less than 2%, less than 1%, or less than 0.1%.

In some embodiments, the purified amphiphilic peptide has a residual Trifluoroacetic acid (TFA) concentration of less than about 1% w/v.

In some embodiments, the purified amphiphilic peptide has a residual acetonitrile concentration of less than about 410 ppm.

In some embodiments, the purified amphiphilic peptide has a residual N,N-Dimethylformamide concentration of less than about 880 ppm.

In some embodiments, the purified amphiphilic peptide has a residual triethylamine concentration of less than about 5000 ppm.

In some embodiments, the purified amphiphilic peptide has a residual Ethyl Ether concentration of less than about 1000 ppm.

In some embodiments, the purified amphiphilic peptide has a residual isopropanol concentration of less than about 100 ppm.

In some embodiments, the purified amphiphilic peptide is lyophilized.

In some embodiments, purified amphiphilic peptide has a net charge of from −9 to +9, for example, +5 to +9.

In some embodiments, the purified amphiphilic peptide has between 70% w/v and 99.9% w/v nitrogen.

In some embodiments, the purified amphiphilic peptide has a bacterial endotoxin level of less than about 10 EU/mg.

In some embodiments, the purified amphiphilic peptide has a water content of between about 1% w/v and about 15% w/v.

In some embodiments, the buffer further comprises water, an acid, a base, a mineral, or any combination thereof.

In some embodiments, the buffer has a substantially physiological pH.

In some embodiments, the buffer comprises from about 5 mM to about 200 mM of the ionic salts.

In some embodiments, the ionic salt dissociates into at least one of sodium, potassium, calcium, magnesium, chloride, and sulfate ions.

In some embodiments, the ionic salts comprise sodium chloride, ammonium chloride, magnesium chloride, potassium chloride, calcium chloride, ammonium sulfate, magnesium sulfate, sodium sulfate, potassium sulfate, calcium sulfate, or combinations thereof.

In some embodiments, the buffer comprises from about 10 mM to about 150 mM sodium chloride.

In some embodiments, the buffer comprises from about 1 mM to about 150 mM of a biological buffering agent.

In some embodiments, the biological buffering agent is selected from Bis-tris propane (BTP), 4-(2-hydroxyethyl)-1-piperazineethanesulfonic acid (HEPES), Dulbecco's Modified Eagle Medium (DMEM), tris(hydroxymethyl)aminomethane (TRIS), 2-(N-Morpholino)ethanesulfonic acid hemisodium salt, 4-Morpholineethanesulfonic acid hemisodium salt (MES), 3-(N morpholino)propanesulfonic acid (MOPS), and 3-(N-morpholino)propanesulfonic acid (MOBS), Tricine, Bicine, (tris(hydroxymethyl)methylamino)propanesulfonic acid (TAPS), N-(2-Acetamido)-2-aminoethanesulfonic acid (ACES), β-Hydroxy-4-morpholinepropanesulfonic acid, 3-Morpholino-2-hydroxypropanesulfonic acid (MOPSO), (N,N-bis(2-hydroxyethyl)-2-aminoethanesulfonic acid) (BES), and combinations thereof.

In some embodiments, the buffer comprises from about 10 mM to about 100 mM BTP.

In some embodiments, the predetermined secondary structure comprises a structure preselected from at least one of a β-strand, β-sheet, an α-helix, and a random coil.

In some embodiments, the preselected structure comprises a β-hairpin.

In some embodiments, the folding group is configured to adopt a β-hairpin secondary structure.

In some embodiments, the purified amphiphilic peptide further comprises a bioactive functional group.

In some embodiments, the bioactive functional group has between 3 and 30 amino acid residues.

In some embodiments, the bioactive functional group is engineered to control or alter charge of the peptide.

In some embodiments, the bioactive functional group has a sequence selected from RGD, IKVAV, YIGSR, LKKTETQ, SNKPGVL, PKPQQFFGLM, GKLTWQELYQLKYKGI, GGG, and combinations thereof.

In some embodiments, the purified amphiphilic peptide further includes a modification selected from a linker, a spacer, and combinations thereof.

The preparation may comprise from about 0.5% by weight to about 10% by weight of the biocompatible polymer.

In some embodiments, the biocompatible polymer has a molecular weight of less than about 50 kDa.

In some embodiments, the biocompatible polymer is at least partially biodegradable, non-biodegradable, or combinations thereof.

In some embodiments, the at least two functional groups of the biocompatible polymer are independently selected from an amine, carboxyl, hydroxyl, thiol, succinimidyl ester, alkene, transcyclooctene, alkyne, ring-strained alkyne, dibenzylcyclooctyne, tetrazine, azide, maleimide, and combinations thereof.

In some embodiments, the biocompatible polymer comprises a homobifunctional linear polymer, a heterobifunctional linear polymer, a homofunctional branched polymer, a heterfunctional branched polymer, a homofunctional star polymer, a heterfunctional star polymer, a homofunctional dendritic polymer, a heterofunctional dendritic polymer, a copolymer, a random copolymer, a block copolymer, a diblock compolymer, a triblock copolymer, or combinations thereof.

In some embodiments, the biocompatible polymer is selected from a polyethylene glycol (PEG), derivative thereof or peptide conjugate thereof, polyethylene glycol-poly(lactide-co-glycolide) copolymer (PEG-PLGA), poly(ethylene glycol)-co-poly(glycolic acid) copolymer (PEG-co-PGA), polyvinyl alcohol (PVA), derivative thereof or peptide conjugate thereof, poly(2-hydroxyethyl methacrylate) (PHEMA), poly(N-isopropylacrylamide) (PNIPAAm), poly(acrylic acid) (PAAc), polyurethane, poloxamer, pluronics, polyurethane, polysaccharide, cellulose, carboxymethylcellulose, dextran, oxidized dextran, alginate, oxidized alginate, hyaluronic acid, chitosan, gelatin, elastin, collagen, carob gum, pullulan, and combinations thereof.

In some embodiments, the biocompatible polymer is selected to provide a controllable mechanical structure to the film.

In some embodiments, the at least one chemical crosslinker molecule comprises glutaraldehyde.

The film may comprise from about 0.1% by volume to about 2% by volume of the glutaraldehyde.

The film may have a concentration of glutaraldehyde effective to control gelation kinetics of the film.

In some embodiments, the at least one chemical crosslinker molecule comprises genipin.

In some embodiments, the at least one chemical crosslinker molecule is a photo-crosslinker.

In some embodiments, the coupling chemical agent comprises an amide bond forming agent, a carbodiimide activation agent, a click chemistry agent, a copper-free click chemistry agent, a Michael-type addition agent, or combinations thereof.

In some embodiments, the crosslinking is configured to reach completion in less than about 60 minutes, for example, less than about 30 minutes, less than about 15 minutes, less than about 10 minutes, or less than about 5 minutes, less than about 2 minutes, less than about 60 seconds, less than about 30 seconds, less than about 10 seconds, less than about 3 seconds, or less than about 1 second.

In some embodiments, the biocompatible peptide may be hydrophilic.

In some embodiments, the biocompatible peptide may be hydrophobic.

In some embodiments, the preparation may be formulated as a film.

In some embodiments, the film may have a loss modulus (G′) of from about 0.1 Pa to about 10,000 Pa.

The film may have a storage modulus (G′) of from about 0.1 Pa to about 10,000 Pa.

The film may have a thickness of from about 0.1 mm to about 100 mm when hydrated.

The film may have a thickness of from about 5 μm to 1000 μm when dehydrated.

The film may be at least partially nanoporous, microporous, macroporous, or combinations thereof.

The film may have at least 75% pores by volume.

The film may comprise interconnected pores.

The film may be at least partially cryogelated, lyophilized, or combinations thereof, effective to control porosity.

The film may be formulated for topical, buccal, or parenteral administration.

The film may be formulated for treatment of a microbial infection or elimination or inhibition of proliferation of a target microorganism.

In some embodiments, the target microorganism is a pathogenic microorganism.

The film may be formulated for management or inhibition of a microbial bioburden.

The film may be formulated for treatment of a fungal infection.

The film may be formulated for treatment of a viral infection.

The film may be formulated for treatment of a bacterial infection.

The film may be formulated for treatment of infected wounds and/or treatment or inhibition of biofilm.

The film may be formulated for wound and/or biofilm management.

The film may be formulated for tissue hydration, moisture management, and/or exudate management of wounds or tissues.

The film may be formulated to enable cell attachment and/or tissue adhesion.

The film may be formulated to enable tissue regeneration.

The film may be formulated to enable cell and tissue infiltration within at least 3 days of administration.

In some embodiments, at least 5% by volume of the film is formulated to allow infiltration by cells.

The film may be formulated as a barrier, barrier dressing, and/or hemostat.

In some embodiments, the film may further comprise an active agent, for example, at least one of: an antibacterial composition, an antifungal composition, an antiviral composition, a hemostat, a growth factor, a cytokine, a chemokine, an anti-inflammatory composition, an analgesic composition, a local anesthetic composition, or a pain-relief composition.

The film may be thermally stable between −20° C. and 150° C.

The film may be sterilized by terminal and/or autoclave sterilization.

The film may have a shelf-life of at least about 1-5 years at room temperature.

In some embodiments, the film is physically stable, chemically stable, biologically stable, and/or non-biodegradable.

In some embodiments, the film is biodegradable.

The film may be biodegradable by hydrolysis, proteolysis, or combinations thereof.

In some embodiments, the film is in a hydrated state.

In some embodiments, the hydrated state is at least 85% water by volume.

In some embodiments, the film is capable of being dehydrated and rehydrated.

In some embodiments, the film is in a cryogel state.

In some embodiments, the cryogel state is at least 90% water by volume.

In some embodiments, the film is in a dehydrated state.

In some embodiments, the dehydrated state is less than 25% water by volume.

In some embodiments, the film is capable of rehydration by interaction with a physiological fluid.

The preparation may be substantially free of a preservative.

The preparation may be substantially biocompatible.

The film may be capable of being hydrated in situ.

The film may have a swelling ratio of at least 10-fold.

The film may have a gel fraction of at least 70%.

The film may be capable of at least 10% strain.

The film may have an interpenetrating network hydrogel. The interpenetrating network hydrogel may comprise a first network having covalent crosslinking coupled to a second network having non-covalent crosslinking.

In accordance with another embodiment, there is provided a kit for producing a film. The kit may comprise a biocompatible polymer having at least two functional groups capable of undergoing covalent crosslinking with a peptide group. The kit may comprise a purified amphiphilic peptide comprising a folding group having a plurality of charged amino acid residues and hydrophobic amino acid residues arranged in a substantially alternating pattern and a turn sequence, and at least one functional group available for covalent crosslinking. The kit may comprise a buffer comprising an effective amount of an ionic salt to induce the purified amphiphilic peptide to form a predetermined secondary structure. The kit may comprise instructions to form the film by covalently crosslinking the purified amphiphilic peptide with the biocompatible polymer by a chemical crosslinker molecule or a coupling chemical agent.

In accordance with another aspect, there is provided a kit for producing a film. The kit may comprise a biocompatible polymer. The biocompatible polymer may be capable of undergoing ionic or physical crosslinking with a peptide group. The kit may comprise a purified amphiphilic peptide comprising a folding group having a plurality of charged amino acid residues and hydrophobic amino acid residues arranged in a substantially alternating pattern and a turn sequence, and at least one functional group available for crosslinking. The kit may comprise a buffer comprising an effective amount of an ionic salt to induce the purified amphiphilic peptide to form a predetermined secondary structure. The kit may comprise instructions to form the film by ionically or physically crosslinking the purified amphiphilic peptide with the biocompatible polymer.

In some embodiments, the kit may further comprise instructions to induce the purified amphiphilic peptide to form the predetermined secondary structure by combining the purified amphiphilic peptide with the buffer.

In accordance with another aspect, there is provided a method of producing a film. The method may comprise covalently crosslinking a purified amphiphilic peptide and a biocompatible polymer with a chemical crosslinker molecule or a coupling chemical agent. In some embodiments, the biocompatible polymer has at least two functional groups capable of undergoing covalent crosslinking with a peptide group, and the purified amphiphilic peptide comprises a folding group having a plurality of charged amino acid residues and hydrophobic amino acid residues arranged in a substantially alternating pattern and a turn sequence, and at least one functional group available for covalent crosslinking.

In accordance with another aspect, there is provided a method of producing a film. The method may comprise ionically or physically crosslinking a purified amphiphilic peptide and a biocompatible polymer. In some embodiments, the biocompatible polymer is capable of undergoing ionic or physical crosslinking with a peptide group, and the purified amphiphilic peptide comprises a folding group having a plurality of charged amino acid residues and hydrophobic amino acid residues arranged in a substantially alternating pattern and a turn sequence, and at least one functional group available for crosslinking.

In some embodiments, the method may comprise crosslinking the purified amphiphilic peptide and the biocompatible polymer in situ.

In some embodiments, the method may further comprise inducing the purified amphiphilic peptide to form a predetermined secondary structure by combining the purified amphiphilic peptide with a buffer comprising an effective amount of an ionic salt.

In some embodiments, the method may comprise inducing the purified amphiphilic peptide to form the predetermined secondary structure in situ.

In some embodiments, the method may comprise depositing a first layer comprising the purified amphiphilic peptide and depositing a second layer comprising the biocompatible polymer adjacent the first layer.

The disclosure contemplates all combinations of any one or more of the foregoing aspects and/or embodiments, as well as combinations with any one or more of the embodiments set forth in the detailed description and any examples.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A includes schematic and microscopic images of an assembled peptide hydrogel matrix with encapsulated cells as compared to collagen, according to one embodiment;

FIG. 1B includes a schematic drawing of a mixing device and a schematic representation of cells in a hydrogel matrix, according to one embodiment;

FIG. 2 includes images of sustained therapeutic activity of the administered peptide hydrogel compared to conventional polymer, according to one embodiment;

FIG. 3 is a microscopy image of positively charged peptide hydrogels, according to one embodiment;

FIG. 4 is a graph showing the antimicrobial activity of the peptide hydrogel, in accordance with one embodiment;

FIG. 5 includes images of a mouse model post-burn injury with bacterial infection showing antimicrobial activity of the peptide hydrogel, in accordance with one embodiment;

FIG. 6 is a photograph of the preparation provided in an end-use container, according to one embodiment;

FIG. 7A is a graph of static light scattering (SLS) at 266 nm of exemplary peptides as a function of temperature, according to some embodiments;

FIG. 7B is a graph of static light scattering (SLS) at 266 nm of exemplary peptides as a function of temperature, according to some embodiments;

FIG. 8 includes graphs showing absorbance of a peptide hydrogel as a function of peptide concentration, according to one embodiment;

FIG. 9 is a graph showing net charge of a peptide preparation as a function of pH value, according to one embodiment;

FIG. 10 is a visual representation of net peptide charge at a pH of 7.4 for several amino acid residues, according to one embodiment;

FIGS. 11A-11F are graphs showing modulus for varying film formulations, according to certain embodiments;

FIGS. 12A-12D are graphs showing physicochemical properties of varying film formulations, optionally under varying conditions, according to certain embodiments;

FIGS. 13A-13B are graphs showing antimicrobial activity of varying film formulations, according to certain embodiments;

FIG. 14 includes cytotoxicity information of the film formulation, according to certain embodiments;

FIG. 15 includes microscopic images of DAPI stained cells with several film formulations, according to certain embodiments;

FIG. 16 is a graph showing inflammation score of several film formulations, in accordance with certain embodiments;

FIGS. 17A-17B include microscopic images showing implanted material in mouse models, according to certain embodiments; and

FIG. 18 is a graph showing biodegradability of film formulations, according to certain embodiments.

### DETAILED DESCRIPTION

Preparations comprising self-assembling peptide hydrogels are disclosed herein. The self-assembled peptide may be amphiphilic. The peptide may generally have a folding group having a plurality of charged amino acid residues and hydrophobic residues arranged in a substantially alternating pattern. The peptide may include functional groups to provide desired physical or chemical properties upon administration. The purified peptide may include counterions that improve biocompatibility of the preparation. The counterions may control the self-assembly, physical and chemical properties of the peptide. The counterions may enhance the therapeutic functional properties of the peptide. The preparation may include the peptide in an aqueous biocompatible solution. The preparation may include a buffer solution capable of inducing self-assembly of the peptide upon contact. The buffer solution may contain a buffering agent and ionic salts. The buffer solution composition may be designed to control the assembled hydrogel's physical or chemical properties. The preparation may be designed to be thermally stable.

In general, the preparation may have shear-thinning properties and a substantially physiological pH level. The self-assembled hydrogel may have antimicrobial, antiviral, and/or antifungal properties. The preparation may be administered topically or parenterally. The preparation may be administered for tissue engineering applications. Certain exemplary applications include cell delivery, cell culture, treatment and prevention of fungal infections, treatment and prevention of bacterial infections, wound healing, biofilm treatment, biofilm management, and prevention of biofilm and wound infection, including infection of chronic wounds. Other tissue engineering applications are within the scope of the disclosure.

Methods of administering the preparation to a subject are disclosed herein. The methods may generally include selecting a target site for administration and administering the preparation to the target site. Methods of administering the preparation may also include mixing the preparation with a buffer configured to induce self-assembly of the peptide to form the hydrogel and administering the hydrogel to the target site. In certain exemplary embodiments, the preparation and/or hydrogel may be administered by spray, aerosol, dropper, tube, ampule, instillation, injection, or syringe.

In certain embodiments, methods of administering cells to a subject are disclosed herein. The methods may generally include suspending the cells in a solution comprising a self-assembling peptide and administering an effective amount of the suspension to a target site of the subject. The methods may comprise combining the solution with a buffer configured to induce self-assembly of the peptide. The solution may be combined with the buffer prior to administration, concurrently with administration, or after administration. The buffer may generally comprise an effective amount of an ionic salt and a biological buffering agent.

Unlike other peptides in aqueous solution, the peptides disclosed herein undergo self-assembly. The self-assembly may enable the peptides to be administered in a concentrated or localized manner to a target tissue. For example, self-assembling peptides may be administered at higher concentrations when compared to free floating peptides. The self-assembling peptides may exhibit the clinical benefit of reducing offsite toxicity of the peptides, due to the localizing effect upon administration. Additionally, the therapeutic dosage of peptides may be increased in the vicinity of the target administration site.

Unlike other polymers in aqueous solution, the peptides disclosed herein may undergo self-assembly in situ at the target site. The in situ self-assembly may enable the peptides to be administered to a target tissue and allow to physically or ionically crosslink, for example, within seconds of administration. For example, self-assembling peptides may be administered directly to target site. Conventional free-floating peptides or polymers usually need a crosslinking agent or exogenous added covalent crosslinking agent. Thus, the self-assembling peptides disclosed herein may provide the clinical benefit of reducing product application and complexity. Additionally, the ionic crosslinking of peptides upon self-assembly may provide the benefit of selecting between product removal and permanent adherence to a target administration site.

### Select Definitions

Hydrogels are a class of materials that have significant promise for use in soft tissue and bone engineering. The general characteristic of hydrogels that make them important materials for these applications are their well hydrated, porous structure. Hydrogels may be designed to be compatible with the adhesion and proliferation of various cell types, e.g., fibroblasts and osteoblasts, making them potential tissue engineering scaffolds for generating connective tissue, such as cartilage, tendons, and ligaments, and bone.

The hydrogel material may be cytocompatible. Cytocompatibility, defined herein, means that the hydrogel must not be adverse to desired cells, in vitro and/or in vivo. Adversity to cells may be measured by cytotoxicity, cell adhesion, proliferation, phenotype maintenance, and/or differentiation of progenitor cells.

The hydrogel material may be biocompatible. “Biocompatible,” defined herein, means that a material does not cause a significant immunological and/or inflammatory response if placed in vivo. Biocompatibility may be measured according to International Organization for Standardization (ISO) 10993 standards.

The hydrogel material may be biodegradable affording non-toxic species. The hydrogel material may be proteolytically biodegradable. “Proteolytic” biodegradation, defined herein, refers to local degradation of the material in response to the presence of cell-derived proteases and/or gradual degradation with the proliferation of cells. The hydrogel material may be hydrolytically biodegradable. “Hydrolytic” biodegradation, defined herein, refers to polymer degradation without assistance from enzyme under biologic conditions.

The hydrogel material may be bioresorbable. Bioresorbable, defined herein, means that the hydrogel material breaks down into remnants that are natural products readily absorbed into the body, resulting in complete loss of original mass.

The hydrogel material may be shear-thinning. “Shear-thinning,” as described herein, refers to a variable apparent viscosity, in particular, a decreasing viscosity with increasing applied stress. For instance, the shear-thinning hydrogel may exhibit non-Newtonian fluid properties. In particular the hydrogels disclosed herein may be administered through a needle or catheter and rapidly resume gelation after removal of the mechanical force.

The hydrogel and/or other materials disclosed herein may be referred to as having one or more physiological properties. As disclosed herein, physiological properties or values refer to those which are compatible with the subject. In particular, physiological properties or values may refer to those which are compatible with a particular target tissue. In certain embodiments, physiological properties or values may refer to those which are substantially similar to the properties or values of the target tissue. Physiological properties may include one or more of pH value, temperature, net charge, water content, stiffness, and others.

“Self-assembling” peptides include such peptides which, typically, after being exposed to a stimulus, will assume a desired secondary structure. The peptides may self-assemble into a higher order structure, for example a three-dimensional network and, consequently, a hydrogel. The self-assembled hydrogel may contain peptides in a tertiary and/or quaternary structure through charge screening, hydrophobic, and disulfide interactions. Peptides have been observed to self-assemble into helical ribbons, nanofibers, nanotubes and vesicles, surface-assembled structures and others. Self-assembling peptides may assemble responsive to certain environmental conditions, e.g., pH, temperature, net charge, exposure to light, applied sound wave, or presence or absence of environmental factors. The environmental conditions may occur upon administration to a subject or by combination with a buffer. In other embodiments, the peptide may assemble spontaneously in solution under neutral pH level. The peptide may assemble spontaneously in solution under physiological conditions and/or in the presence of a cation and/or anion.

The self-assembling peptides may assemble into an alpha helix, pi-helix, beta sheet, random coil, turn, beta pleated parallel, antiparallel, twist, bulge, or strand connection secondary structure and combinations of thereof. For example, a 20 amino acid peptide which self-assembles into β-strands may comprise alternating valine and lysine residues flanking a tetrapeptide sequence (-VDPPT-). When dissolved in low ionic strength and buffered aqueous solution, the exemplary peptide resides in an ensemble of random coil conformers due to electrostatic repulsions of the positively charged lysine residues. Upon increasing the ionic strength and/or pH of the solution, the lysine-based positive charge is relieved due to either screening of the charge or deprotonating a sufficient amount of the side chain amines. This exemplary action enables peptide folding into an amphiphilic β-hairpin. In the folded state, the exemplary peptide self-assembles via lateral and facial associations of the hairpins to form a non-covalently crosslinked hydrogel containing β-sheet rich fibrils. Thus, the self-assembling peptides may be designed to undergo hydrogelation under varying conditions through rational design of the peptide sequence.

The self-assembling peptides disclosed herein may assemble into a nano-porous tertiary structure. As disclosed herein, the nano-porous structure is a three-dimensional matrix containing pores having an average size of 1-1000 nm. The pores or voids may constitute between 10% and 90% of the three-dimensional matrix by volume. For example, the pores or voids may constitute 10%, 20%, 30%, 40%, 50%, 60%, 70%, 80%, or 90% of the three-dimensional matrix by volume. The pores may be permeable and allow diffusion of liquid and/or gas. The nano-porous structure is constructed by physical crosslinks, allowing ionic bonds to be broken and reformed upon asserted stress. These nano-porous structure may allow for cells to attach and/or migrate through the matrix. The nano-porous structure may also mimic the endogenous extra-cellular matrix environment of tissues and, optionally, be selected to mimic a specific tissue.

“Disassembly” of the peptides may refer to the ability of the peptide to assume a lower order structure after being exposed to a stimulus. Disassembly may also refer to the ability of the physically crosslinked peptide to temporarily break hydrophobic and disulfide bonds to assume a lower order structure after being exposed to a stimulus. For example, a tertiary structure protein may disassemble into a secondary structure protein, and further disassemble into a primary structure peptide. In accordance with certain embodiments, self-assembly and disassembly of the peptide may be reversible.

Preparations and formulations disclosed herein may generally be referred to as peptide preparations. The peptide preparations may include a self-assembling peptide and/or a self-assembled hydrogel as disclosed herein. The peptide preparation may include a cytocompatible and/or biocompatible solution. The preparation may include a buffer. While reference is made to a solution, it should be understood that the preparation may be in the form of a liquid, gel, or solid particle. In certain embodiments, for example, the preparation may be in the form of the assembled hydrogel. In other embodiments, the preparation may be in the form of a lyophilized powder.

The peptide preparation may further include one or more bioactive components for tissue engineering, such as, functionalized peptides, cells, media, serum, collagen and other structure-imparting components, antibodies and antigens, bioactive small molecules, and other bioactive drugs. “Bioactivity” as described herein refers to the ability of a compound to impart a biological effect.

Cell containing preparations and formulations disclosed herein may be referred to as cell suspensions. Cell suspensions include a plurality of cells, e.g., living cells, suspended in a solution. The solution may be or comprise water, media, or buffer. The suspension may generally further comprise a self-assembling peptide and/or a self-assembled hydrogel, as disclosed herein. While reference is made to cells, it should be understood that the suspension may contain cell fragments and/or tissue, e.g. tissue grafts, in addition to or instead of the cells. For example, the suspension may contain live or dead cells or cell fragments, spheroids, and/or cell aggregates.

The cells may be isolated from living tissue and subsequently maintained and/or grown in cell culture. The cell culture conditions may vary, but generally include maintaining the cells in a suitable vessel with a substrate or medium that supplies the essential nutrients, e.g., amino acids, carbohydrates, vitamins, minerals, growth factors, hormones, and gases, e.g., CO₂ and O₂, and regulating the physio-chemical environment, e.g., pH, osmotic pressure, temperature. The cells may be maintained in live cell lines, e.g., a population of HeLa cells descended from a single cell and containing the same genetic makeup.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, “treatment” of an injury, condition, or disease refers to reducing the severity or frequency of at least one symptom of that injury, condition, or disease, compared to a similar but untreated subject. Treatment can also refer to halting, slowing, or reversing the progression of an injury, condition, or disease, compared to a similar but untreated subject. Treatment may comprise addressing the root cause of the injury, condition, or disease and/or one or more symptoms. “Management” of an injury, condition, or disease may refer to reducing the severity or frequency of at least one symptom of that injury, condition, or disease, to a tolerable level, as determined by the subject or a health care provider.

As used herein an effective amount refers to a dose sufficient to achieve a desired result. For example, the effective amount may refer to a concentration sufficient to achieve self-assembly of the hydrogel and/or provide desired properties. An effective amount may refer to a dose sufficient to prevent advancement, or to cause regression of an injury, condition, or disease, or which is capable of relieving a symptom of an injury, condition, or disease, or which is capable of achieving a desired result. An effective amount can be measured, for example, as a concentration of peptide or other component in the preparation, solution, or buffer. An effective amount can be measured, for example, as a concentration of bioactive agent or an effect or byproduct of a bioactive agent. An effective amount can be measured, for example, as a number of cells or number of viable cells, or a mass of cells (e.g., in milligrams, grams, or kilograms), or a volume of cells (e.g., in mm³).

Throughout this disclosure, formulation may refer to a composition or preparation or product.

Administered “in combination,” as used herein, means that two (or more) different treatments are delivered to the subject during the course of the subject's affliction with the injury, e.g., the preparation is delivered with a second agent after the subject has been diagnosed with the condition or injury and before the condition or injury has been cured or eliminated. In certain embodiments, administration in combination means the preparation additionally comprises one or more second agent. In some embodiments, the delivery of one treatment is still occurring when the delivery of the second begins, so that there is overlap. This is sometimes referred to herein as “simultaneous” or “concomitant” or “concurrent delivery.” In other embodiments, the delivery of one treatment ends before the delivery of the other treatment begins. This is sometimes referred to herein as “successive” or “sequential delivery.”

In embodiments of either case, the treatment is more effective because of combined administration. For example, the second agent is a more effective, e.g., an equivalent effect is seen with less of the second agent, or the second agent reduces symptoms to a greater extent, than would be seen if the second agent were administered in the absence of the preparations disclosed herein, or the analogous situation is seen with the preparation. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the administration of the preparation is still detectable when the second agent is delivered. In some embodiments, one or more treatment may be delivered prior to diagnosis of the patient with the injury.

As used herein, a subject may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others. The term “non-human animals” includes research animals, for example, for example, mouse, rat, rabbit, dog, cat, pig, among others.

### Properties of the Peptide Sequence and Secondary Structure

The peptides disclosed herein may have a sequence configured to fold into a desired secondary structure. The secondary structure may refer to a three-dimensional form of local segments of proteins. The secondary structure may comprise, for example, pleated sheet, helical ribbon, nanotube and vesicle, surface-assembled structure, and others. The peptides disclosed herein may have a sequence configured to self-assemble into a desired tertiary structure. The tertiary structure may refer to a three-dimensional organization of secondary structure protein forms. The tertiary structure may comprise, for example, three-dimensional matrix, porous matrix, nano-porous matrix.

Self-assembling peptides disclosed may be designed to adopt a secondary, for example, β-hairpin, and/or tertiary structure in response to one or more signals. Typically, after adopting the secondary structure, the peptides will self-assemble into a higher order structure, e.g., a hydrogel. In certain embodiments, the self-assembly does not take place unless side chains on the peptide molecules are uniquely presented in the secondary structure conformation. The self-assembling peptides may assemble responsive to certain environmental conditions, e.g., pH, temperature, net charge, exposure to light, applied sound wave, or presence or absence of environmental factors. The environmental conditions which induce self-assembly may occur upon administration to a subject, e.g., upon contact with a target tissue. In some embodiments, the environmental conditions which induce self-assembly may occur upon combination of the peptide preparation with a buffer configured to induce self-assembly. The buffer may have a pH or composition configured to induce self-assembly. For example, the buffer may have a concentration of ions configured to induce self-assembly.

Self-assembly of the peptides disclosed herein may produce compact structures that exhibit biophysical structural relationships with the intended function of the peptide. For example, a compact tertiary structure may have a higher number of active amino acid residues per unit area, compared to unassembled peptides. In the particular example of antimicrobial peptides, the tertiary structure may enable a higher concentration of charged, e.g., positively charged, amino acid residues per area, increasing antimicrobial properties (e.g., bacterial membrane destabilization and disruption).

In certain embodiments, the self-assembling peptide hydrogels may include those disclosed in and/or prepared by the methods disclosed in any of U.S. Pat. Nos. 8,221,773; 7,884,185; 8,426,559; 7,858,585; and 8,834,926, incorporated herein by reference in their entireties for all purposes. For example, the self-assembling peptide hydrogels may be or comprise any of SEQ ID NOS: 1-20 from U.S. Pat. Nos. 8,221,773, 7,884,185, and 7,858,585; and SEQ ID NOS: 1-33 from U.S. Pat. No. 8,834,926. Other self-assembling peptides are known and may be employed to bring about the methods disclosed herein.

The desired properties of the self-assembling peptides may be controlled by peptide design. The self-assembling peptides may be small peptides, e.g., from about 6 to about 200 residues or from about 6 to about 50 residues or from about 10 to about 50 residues. Any of the amino acid residues may be a D isoform. Any of the amino acid residues may be an L isoform.

Self-assembling peptides disclosed herein may be designed to be substantially amphiphilic when assembled into the tertiary structure. “Amphiphilic” molecules, e.g., macromolecules or polymers, as disclosed herein, typically contain hydrophobic and hydrophilic components. Peptide amphiphiles are one exemplary class of amphiphilic molecules. Peptide amphiphiles are peptide-based molecules that typically have the tendency to self-assemble into high-aspect-ratio nanostructures under certain conditions. The exemplary conditions may comprise selected pH, temperature, and ionic strength values. One particular type of peptide amphiphiles comprise alternating charged, neutral, and hydrophobic residues, in a repeated pattern, for example, as disclosed herein. A combination of intermolecular hydrogen bonding and hydrophobic and electrostatic interactions may be designed to form well-defined self-assembled nanostructures by assembly of the disclosed peptide amphiphiles.

The self-assembling peptides may include additional amino acids, for example, an epitope. For example, the self-assembling peptides may include additional functional groups, optionally selected by peptide design. Exemplary functional groups disclosed herein comprise a biologically derived motif, for example, having an effect on biological processes such as cell signal transduction, cell adhesion in the extra-cellular matrix (ECM), cell growth, and cell mobility. The peptide may include one or more modifications, for example, a linker or spacer.

In some embodiments, at least one of the N-terminus and the C-terminus may be modified. For example, at least one of the N-terminus and the C-terminus may be amidated. At least one of the N-terminus and the C-terminus may comprise a cysteine moiety. At least one of the N-terminus and the C-terminus may be acetylated. In certain exemplary embodiments, the C-terminus may be amidated and/or the N-terminus may be acetylated. In some embodiments, at least one of the N-terminus and the C-terminus may be free.

One exemplary amidated self-assembling peptide is CVKVRVRVRV(d)PPTRVRVRV KV—NH₂. Any of the peptides disclosed herein may be amidated. One exemplary acetylated and amidated self-assembling peptide is Ac—CVKVRVRVRV(d)PPTRVRVRVKVC—NH₂. Any of the peptides disclosed herein may be amidated, acetylated, or both.

In general, the self-assembling peptides may have a folding group configured to adopt the secondary and/or higher order structure. Exemplary self-assembling peptides may have a folding group designed to adopt a β-hairpin secondary structure. Exemplary self-assembling peptides may have a folding group designed to adopt a three-dimensional nano-porous matrix tertiary structure. Self-assembling peptides disclosed herein may be designed to adopt a β-hairpin secondary structure and/or nano-porous matrix tertiary structure in response to one or more environmental stimulus at the target site, e.g., at a topical or parenteral site. The self-assembling peptides may also be designed to self-assemble into a range of other self-assembled structures, such as spherical micelles, vesicles, bilayers (lamellar structures), nanofibers, nanotubes, and ribbons.

The self-assembly folding group may have between about 2 and about 200 residues, for example, between about 2 and about 50 residues, between about 10 and about 30 residues, between about 15 and about 25 residues, for example, about 20 residues.

In accordance with some embodiments, the self-assembling folding group may include hydrophobic amino acids. “Hydrophobic” amino acid residues are those which tend to repel water. Such hydrophobic amino acids may include glycine, alanine, valine, leucine, isoleucine, proline, phenylalanine, methionine, threonine, and tryptophan. In certain embodiments, the hydrophobic amino acid residues may comprise valine.

The folding group may be functionalized by addition of other functional residues as described herein, or conserved for self-assembly. Exemplary functional residues include basic, neutral, aliphatic, aromatic, and polar amino acid residues.

The folding group may have a plurality of basic, neutral, aliphatic, aromatic, polar, charged amino acid residues. The folding group may have a plurality of hydrophobic amino acid residues arranged in a substantially alternating pattern with non-hydrophobic amino acid residues. In certain embodiments, the folding group may have a plurality of hydrophobic amino acid residues arranged in a substantially alternating pattern with a plurality of charged amino acid residues.

The folding group may comprise a turn sequence. The turn sequence may include one or more internal amino acid residues within the folding group. In certain embodiments, the turn sequence may be substantially centrally located within the folding group.

The turn sequence may have between about 2 and about 20 residues, for example, between about 2 and about 10 residues, between about 2 and about 8 residues, between about 2 and about 5 residues, for example, about 2 residues, about 3 residues, about 4 residues, or about 5 residues.

In exemplary embodiments, the turn sequence may include one or more of proline, aspartic acid, threonine, and asparagine. The turn sequence may include D-proline and/or L-proline. In some embodiments, the turn sequence may have 1-4 proline residues, for example, 1 proline residue, 2 proline residues, 3 proline residues, or 4 proline residues.

Exemplary self-assembling peptides may have a folding group sequence comprising [AY]_{N}[T][YA]_{M}, where A is 1-3 amino acids selected from one or more of basic, neutral, aliphatic, aromatic, polar, and charged amino acids, Y is 1-3 hydrophobic amino acids, T is 2-8 turn sequence amino acids, and N and M are each independently between 2 and 10. Y amino acids may independently be selected from glycine, alanine, valine, leucine, isoleucine, proline, phenylalanine, methionine, threonine, and tryptophan. In some embodiments, the folding group sequence may be Y[AY]_{N}[T][YA]_{M}Y—NH₂.

Certain exemplary self-assembling peptides may have a folding group sequence comprising [XY]_{N}[T][YX]_{M}, where X is 1-3 charged amino acids, Y is 1-3 hydrophobic amino acids, T is 2-8 turn sequence amino acids, and N and M are each independently between 2 and 10. X amino acids may independently be selected from arginine, lysine, tryptophan, and histidine. Y amino acids may independently be selected from glycine, alanine, valine, leucine, isoleucine, proline, phenylalanine, methionine, threonine, and tryptophan. In some embodiments, the folding group sequence may be Y[XY]_{N}[T][YX]_{M}Y—NH₂.

Certain exemplary self-assembling peptides may have a folding group sequence comprising [ZY]_{N}[T][YZ]_{M}, where Z is 1-3 polar or charged amino acids, Y is 1-3 hydrophobic amino acids, T is 2-8 turn sequence amino acids, and N and M are each independently between 2 and 10. Z amino acids may independently be selected from glutamine, asparagine, histidine, serine, threonine, tyrosine, cysteine, alanine, valine, leucine, isoleucine, proline, phenylalanine, arginine, lysine, aspartic acid, and glutamic acid. Y amino acids may independently be selected from glycine, alanine, valine, leucine, isoleucine, proline, phenylalanine, methionine, threonine, and tryptophan. In some embodiments, the folding group sequence may be Y[ZY]_{N}[T][YZ]_{M}Y—NH₂.

One exemplary self-assembling peptide may have a folding group comprising -RYRYRYTYRYRYR- where R is an arginine residue, Y is 1-3 hydrophobic amino acids, and T is 2-6 turn sequence amino acids.

One exemplary self-assembling peptide may have a folding group comprising -VXVXVXVXVTXVXVXVXV- where V is a valine residue, X may be independently selected from charged and neutral amino acid residues serine, glutamic acid, lysine, tryptophan, and histidine, and T is 2-8 turn sequence amino acids. In some embodiments, the exemplary folding group may comprise a series of hydrophobic valine amino acid residues alternating with independently selected hydrophilic and/or neutral amino acid residues.

One exemplary self-assembling peptide may have a folding group comprising -KYKYKYTYKYKYK- where R is an arginine residue, Y is 1-3 hydrophobic amino acids, and T is 2-6 turn sequence amino acids.

One exemplary self-assembling peptide may have a folding group comprising -VZVZVZVTVZVZVZV- where V is a valine residue, Z is 1-3 hydrophilic amino acids, and T is 2-6 turn sequence amino acids.

Exemplary self-assembling peptides may have a turn sequence comprising 2-8 turn sequence amino acids, for example 2-5 turn sequence amino acids. The turn sequence amino acids may be selected from proline, for example D-proline and/or L-proline, aspartic acid, and asparagine. In some embodiments, the turn sequence may be (d)PP, (d)PG, or NG.

Exemplary self-assembling peptides having a turn sequence include VKVRVRVRV(d)PPTRVRVRVKV—NH₂ and VLTKVKTKV(d)PPTKVEVKVLV—NH₂. In the exemplary peptides, the tetrapeptide turn sequence (—V(d)PPT-) was selected to adopt a type II′ turn and positioned within the middle of the peptide sequence. This four-residue turn sequence occupies the i, i+1, i+2 and i+3 positions of the turn. The heterochiral sequence ((d)P(i+1)−P (i+2)) dipeptide was selected for its tendency to adopt dihedral angles consistent with type II′ turns. Incorporation of a bulky β-branched residue (valine) at the i position of the turn sequence enforces the formation of a trans prolyl amide bond at the i+1 position. The placement of valine at this position is selected to design against the formation of a cis prolyl bond, which results in β-strands that adopt an extended conformation rather than the intended hairpin. Threonine exhibits a statistical propensity to reside at the i+3 position. Therefore, threonine was selected to be incorporated at this position within the tetrapeptide sequence, which bears a side-chain hydroxyl group capable of hydrogen bonding to the amide backbone carbonyl at the i position, to further stabilize the turn.

The exemplary folding peptides may be designed to include high propensity β-sheet forming residues flanking the type II′ turn sequence. The selection of alternation of hydrophobic and hydrophilic residues along the strands provides an amphiphilic β-sheet when the peptide folds. For example, lysine may be chosen as a hydrophilic residue to provide a side chain pKa value of about 10.5. Side chain amines are generally protonated when dissolved under slightly acid conditions, forming unfavorable electrostatic interactions between β-strands of the hairpin and inhibiting peptide folding and self-assembly. However, as pH is increased to about pH 9, a sufficient portion of the lysine side chains become deprotonated allowing the peptide to fold into an amphiphilic β-hairpin. The electrostatic interactions may be employed to design pH responsiveness of the disclosed peptides.

While not wishing to be bound by theory, it is believed the amphiphilic β-hairpin is stabilized in the intramolecular folded state by van der Waals contacts between neighboring amino acid side chains within the same hairpin. The formation of intramolecular hydrogen bonds between cross β-strands of the hairpin and the propensity for the turn sequence to adopt at type II′ turn may further stabilize the folded conformation. Once in the folded state, the lateral and facial associations of the β-hairpins may be selected to design self-assembly. For example, lateral association of β-hairpins promotes the formation of intermolecular hydrogen bonds and van der Waals contacts between neighboring amino acids.

Exemplary self-assembling peptides may have a folding group sequence comprising (X)a(Y)b(Z)c-[(d)PP, (d)PG, or NG]-(Z)c(Y)b(X)a, where the turn sequence is (d)PP, (d)PG, or NG, (d)P is a D-proline, X is a charged amino acid, Y is a hydrophobic amino acid, Z is a hydrophobic amino acid or a polar amino acid, and a, b, and c are each independently an integer from 1-10. In certain embodiments, X is independently selected from valine, leucine, isoleucine, phenylalanine, tryptophan, tyrosine, threonine, and combinations thereof. In certain embodiments, Y is independently selected from glutamic acid, serine, alanine, proline, aspartic acid, and combinations thereof. In some embodiments, Z is independently selected from glutamine, glutamic acid, lysine, arginine, and combinations thereof.

Exemplary self-assembling peptides may have a folding group sequence comprising (Z)c(Y)b(X)a-[(d)PP, (d)PG, or NG]-(X)a(Y)b(Z)c, where the turn sequence is (d)PP, (d)PG, or NG, (d)P is a D-proline, X is a charged amino acid, Y is a hydrophobic amino acid, Z is a hydrophobic amino acid or a polar amino acid, and a, b, and c are each independently an integer from 1-10. In certain embodiments, X is independently selected from valine, leucine, isoleucine, phenylalanine, tryptophan, tyrosine, threonine, and combinations thereof. In certain embodiments, Y is independently selected from glutamic acid, serine, alanine, proline, aspartic acid, and combinations thereof. In some embodiments, Z is independently selected from glutamine, glutamic acid, lysine, arginine, and combinations thereof.

Any of the charged, hydrophobic, polar, or amphipathic amino acids disclosed herein may derive one or more of their properties from the composition of the biocompatible solution.

Hydrophobic amino acids are those which tend to repel water. Hydrophobic amino acids include alanine, valine, leucine, isoleucine, proline, tyrosine, tryptophan, phenylalanine, methionine, and cysteine. The hydrophobic amino acids may be independently selected from alanine, valine, leucine, isoleucine, proline, phenylalanine, methionine, cysteine, and combinations thereof. In some embodiments, the hydrophobic amino acids comprise valine.

Charged amino acids are those which tend to have an electric charge under the given conditions. Charged amino acids may have side chains which form salt bridges. Charged amino acids include alanine, valine, leucine, isoleucine, proline, phenylalanine, cysteine, arginine, lysine, histidine, aspartic acid, and glutamic acid. The folding group may comprise 2-10 charged amino acids, for example 2-8 charged amino acids.

The charged amino acids may be positively charged amino acids. The folding group may comprise 2-10 charged amino acids, for example 2-8 charged amino acids. The positively charged amino acids may be independently selected from arginine, lysine, histidine, and combinations thereof. The folding group may comprise 2-8 arginine residues, lysine residues, or a combination of arginine and lysine residues. In some embodiments, the folding group may comprise 6 positively charged residues selected from arginine, lysine, or a combination of arginine and lysine.

The charged amino acids may be negatively charged amino acids. The folding group may comprise 2-10 negatively charged amino acids, for example, 2-8 negatively charged amino acids. In some embodiments, the negatively charged amino acids may be independently selected from aspartic acid, glutamic acid, and combinations thereof.

Polar amino acids are those which have an uneven charge distribution. Polar amino acids may tend to form hydrogen bonds as proton donors or acceptors. Polar amino acids include glutamine, asparagine, histidine, serine, threonine, tyrosine, and cysteine.

Amphipathic amino acids are those which have both a polar and non-polar component. Amphipathic amino acids may be found at the surface of proteins or lipid membranes. Amphipathic amino acids include tryptophan, tyrosine, and methionine.

Exemplary self-assembling peptides may have a folding group sequence of any of SEQ ID NOS: 1-23. The self-assembling peptide may have a folding group sequence of SEQ ID NO: 1. In certain embodiments, the self-assembling peptide may have a folding group sequence of SEQ ID NO: 2. The self-assembling peptide may have a folding group sequence of SEQ ID NO: 3. The self-assembling peptide may have a folding group sequence of SEQ ID NO: 4. The self-assembling peptide may have a folding group sequence of SEQ ID NO: 5. The self-assembling peptide may have a folding group sequence of SEQ ID NO: 6. The self-assembling peptide may have a folding group sequence of SEQ ID NO: 7. The self-assembling peptide may have a folding group sequence of SEQ ID NO: 8. The self-assembling peptide may have a folding group sequence of SEQ ID NO: 9. The self-assembling peptide may have a folding group sequence of SEQ ID NO: 10. The self-assembling peptide may have a folding group sequence of SEQ ID NO: 11. The self-assembling peptide may have a folding group sequence of SEQ ID NO: 12. The self-assembling peptide may have a folding group sequence of SEQ ID NO: 13. The self-assembling peptide may have a folding group sequence of SEQ ID NO: 14. The self-assembling peptide may have a folding group sequence of SEQ ID NO: 15. The self-assembling peptide may have a folding group sequence of SEQ ID NO: 16. The self-assembling peptide may have a folding group sequence of SEQ ID NO: 17. The self-assembling peptide may have a folding group sequence of SEQ ID NO: 18. The self-assembling peptide may have a folding group sequence of SEQ ID NO: 19. The self-assembling peptide may have a folding group sequence of SEQ ID NO: 20. The self-assembling peptide may have a folding group sequence of SEQ ID NO: 21. The self-assembling peptide may have a folding group sequence of SEQ ID NO: 22. The self-assembling peptide may have a folding group sequence of SEQ ID NO: 23.

Exemplary self-assembling peptides which have shear-thinning properties include VKVRVRVRV(d)PPTRVRVRVKV—NH₂, and VKVRVRVRV(d)PPTRVEVRVKV—NH₂ (which has a single substitution of glutamic acid at position 15 on the hydrophilic face). The glutamic acid substitution results in a faster rate of gelation of the self-assembling peptide gel in the presence of ionic salts in the biocompatible solution. The negatively charged glutamic acid lowers the overall positive charge of the peptide and enables faster folding and self-assembly.

Exemplary self-assembling peptides which have shear-thinning properties that can be tuned for net peptide charge include VKVRVRVRV(d)PPTRVEVRVKV—NH₂, and VKVKVKVKV(d)PPTKVEVKVKV—NH₂, (which has an arginine substituted for lysine on the hydrophilic face). The lysine substitution lowers the peptide net charge at physiological pH that allows for better mammalian cell cytocompatibility when compared to peptides with high arginine content (higher net charge). The exemplary peptides are antimicrobial self-assembling peptides.

Exemplary self-assembling peptides which have shear-thinning properties that can be tuned for peptide gels with faster rate of gelation and increased stiffness include FKFRFRFRV-(d)PPTRFRFRFKF—NH₂, (which has valine substituted for phenylalanine on the hydrophobic face). The phenylalanine substitution increases the hydrophobic face of the peptide that allows for stiffer and faster gelation of peptide gels. The exemplary peptides are antimicrobial self-assembling peptides.

Exemplary self-assembling peptides which have shear-thinning properties include enantiomer forms of the exemplary sequences listed above, such as an enantiomer form of VKVRVRVRV(d)PPTRVRVRVKV—NH₂, (d)V(d)K(d)V(d)R(d)V(d)R(d)V(d)R(d)V(L)P(d)P (d)T(d)R(d)V(d)R(d)V(d)R(d)V(d)K(d)V—NH₂, (which has D isoforms of the sequence and an L isoform of P). The isoform substitution may provide control of peptide degradation and increased stability without compromising peptide net charge at physiological pH. The sequence may provide better compatibility with mammalian cells. The peptide may be a complete enantiomer (as shown above) or a partial enantiomer such that any one or more of the amino acids may be an enantiomer of the sequences listed above. The exemplary peptides are antimicrobial self-assembling peptides.

Other exemplary self-assembling peptides include Ac-VEVSVSVEV(d)PPTEVSVEVEV GGGGRGDV—NH₂ and VEVSVSVEVdPPTEVSVEVEV—NH₂.

The self-assembling peptide may comprise at least one guanidine moiety. The guanidine moiety may be associated with an organic molecule which makes up part of the peptide chain. In exemplary embodiments, a guanidine group may be incorporated as part of the side chain of an arginine residue. However, the peptide may comprise guanidine moieties which are not associated with arginine residues.

A guanidine moiety is generally a highly polar group which, when positioned on a cationic peptide, may allow for pairing with hydrophobic and hydrophilic groups forming salt bridges and hydrogen bonds. Such a peptide may display a high capacity to penetrate cell membranes and provide antimicrobial activity. The guanidine moiety may also promote peptide stability by improving peptide folding, physical characteristics and thermal stability of the peptide and/or hydrogel.

The peptide may generally have 20-50% guanidium content, as measured by number of guanidine groups by total number of amino acid residues of the peptide. For instance, an exemplary peptide sequence having 20 amino acid residues, of which 6 are arginine residues having a guanidine group, has 30% guanidium content. The exemplary peptides may penetrate and disrupt cell membranes.

### Properties of the Peptide Hydrogel Preparation

The preparation may generally comprise the self-assembling peptide in a biocompatible solution. For example, the peptide may be dissolved or substantially dissolved in the biocompatible solution. The preparation may comprise between about 0.1% w/v and about 10.0% w/v of the peptide. The preparation may be formulated for a target indication. For instance, the concentration of the self-assembling peptide may be selected based on a target indication. For example, an exemplary preparation having antimicrobial properties may comprise less than 1.5% w/v of the peptide, for example, between about 0.5% w/v of the peptide and 1.0% w/v of the peptide.

Exemplary preparations may comprise between about 0.1% w/v and about 10.0% w/v of the peptide, for example, between about 0.15% w/v and about 10.0% w/v of the peptide, between about 0.1% w/v and about 8.0% w/v of the peptide, between about 0.25% w/v and about 6.0% w/v of the peptide, between about 0.5% w/v and about 6.0% w/v of the peptide. When the peptide is purified, the preparation may comprise up to about 6.0% w/v of the peptide. In certain embodiments, the preparation may comprise less than about 3.0% w/v of the peptide, for example, between about 0.25% w/v and about 3.0% w/v of the peptide, between about 0.25% w/v and about 2.0% w/v of the peptide, between about 0.25% w/v and about 1.25% w/v of the peptide, or between about 0.5% w/v of the peptide and about 1.5% w/v of the peptide. The preparation may comprise between about 0.5% w/v and about 1.0% w/v of the peptide, between about 0.7% w/v and about 2.0% w/v of the peptide, or between about 0.7% w/v and about 0.8% w/v of the peptide. For instance, the preparation may comprise about 0.25% w/v, about 0.5% w/v, about 0.7% w/v, about 0.75% w/v, about 0.8% w/v, about 1.0% w/v, about 1.5% w/v of the peptide, about 2.0% w/v, or about 3.0% w/v. In particular embodiments, the preparation may comprise less than about 1.5% w/v of the peptide. The preparation may comprise less than about 1.25% w/v of the peptide or less than about 1.0% w/v of the peptide. In one exemplary embodiment, the preparation may comprise about 0.75% w/v of the peptide.

After combination with the buffer, the hydrogel may have between about 0.05% w/v and 6.0% w/v of the peptide. For example, the hydrogel may have between about 0.1% w/v, and 6.0% w/v of the peptide, between about 0.25% w/v and 6.0% w/v of the peptide, between about 1.5% w/v and 6.0% w/v of the peptide, between about 0.25% w/v and 3.0% w/v of the peptide, between about 0.25% w/v and 1.0% w/v of the peptide, between about 0.25% w/v and 0.5% w/v of the peptide, or between about 0.3% w/v and 0.4% w/v of the peptide. The peptide preparation and buffer may be combined to form the hydrogel at a ratio of between about 2:1 to 0.5:1 peptide preparation to buffer. In some embodiments, the peptide preparation and buffer may be combined to form the hydrogel at a ratio of about 1:1.

The peptides in the preparation may be purified. As disclosed herein, “purified” may refer to compositions treated for removal of contaminants. In particular, the purified peptides may have a composition suitable for clinical application. For example, the peptides may be purified to meet health and/or regulatory standards for clinical administration. The peptide may be at least 80% purified, for example, at least 85%, at least 90%, at least 92%, at least 95%, at least 98%, at least 99%, or at least 99.9%.

In certain embodiments, the peptides may be purified to remove or reduce residual organic solvent content from solid phase synthesis of the peptides. The peptide may comprise less than 20% residual organic solvent by weight. The peptide may comprise less than 15% residual organic solvent by weight. The peptide may comprise less than 10% residual organic solvent by weight. For example, the peptide may comprise less than 8%, less than 5%, less than 2%, less than 1%, or less than 0.1% residual organic solvent by weight. Exemplary organic solvents which may be removed or reduced from the synthesized peptide include trifluoroacetic acid (TFA), acetonitrile, isopropanol, N,N-Dimethylformamide, triethylamine, Ethyl Ether, and acetic acid.

The purified peptides may be substantially free of Trifluoroacetic acid (TFA). For example, the purified peptides may have less than 1% w/v residual TFA, or between about 0.005% w/v and 1% w/v residual TFA.

The purified peptide may be substantially free of acetonitrile. In some embodiments, the purified peptide may have less than about 410 ppm residual acetonitrile. The purified peptide may have between about 0.005 ppm and about 410 ppm residual acetonitrile.

The purified peptide may be substantially free of isopropanol. In some embodiments, the purified peptide may have less than about 400 ppm residual isopropanol. The purified peptide may have less than about 100 ppm residual isopropanol. The purified peptide may have between about 0.005 ppm and 100 ppm residual isopropanol.

The purified peptide may be substantially free of N,N-Dimethylformamide. In some embodiments, the purified peptide may have less than about 880 ppm residual N,N-Dimethylformamide. The purified may have between about 0.005 ppm and about 880 ppm residual N,N-Dimethylformamide.

The purified peptide may be substantially free of triethylamine. In some embodiments, the purified peptide may have less than about 5000 ppm residual triethylamine. The purified peptide may have between about 0.005 ppm and about 5000 ppm residual triethylamine.

The purified peptide may be substantially free of Ethyl Ether. In some embodiments, the purified peptide may have less than about 1000 ppm residual Ethyl Ether. The purified peptide may have between about 0.005 ppm and about 1000 ppm residual Ethyl Ether.

The purified peptides may be substantially free of acetic acid. For example, the purified peptides may have less than 2% w/v residual acetic acid, for example, less than 1% w/v residual acetic acid, less than 0.5% w/v residual acetic acid, less than 0.1% w/v residual acetic acid, between about 0.0001% w/v and 2% w/v residual acetic acid, or between about 0.005% w/v and 0.1% w/v residual acetic acid.

In general, the purified peptide and/or biocompatible solution may have properties consistent with regulatory limits defined by the International Council for Harmonization of Technical Requirements for Pharmaceuticals for Human Use (ICH).

The biocompatible solution of the preparation may refer to a substantially liquid carrier for the peptide. The biocompatible solution may generally be an aqueous solution. The biocompatible solution may comprise water, for example, deionized water. Deionized water may have a resistivity of greater than about 18 MΩ and a conductivity of less than about 0.056 μS at 25° C. Deionized water may have a maximum endotoxin specification of 0.03 endotoxin units (EU)/ml and 1 CFU/mL microbial action or less. Deionized water may have a total organic carbon (TOC) concentration of 10 ppb or less. The biocompatible solution may comprise pharmaceutical grade water. Pharmaceutical grade water may have 500 ppb total organic carbon (TOC) or less and 100 CFU/ml microbial action or less. The biocompatible solution may comprise injection grade water. Injection grade water may have a maximum endotoxin specification of 0.25 endotoxin units (EU)/ml and 10 CFU/10 ml microbial action or less. In certain embodiments, the preparation or biocompatible solution may be substantially free of chloride ions.

The preparation or peptide may comprise counterions. As disclosed herein, a counterion may refer to a charge balancing ion. The preparation or peptide may have an effective amount of counterions to render the preparation substantially electrically neutral. The preparation or peptide may have an effective amount of counterions to render the preparation substantially biocompatible and/or stable. The preparation or peptide may have an effective amount of counterions to control repulsion of anionic or cationic residues of the peptide. The concentration of counterions may be dependent on the peptide sequence and concentration of the peptide and any additives. In exemplary embodiments, the peptide may comprise between 0.1-20% counterions. Additionally, the charge of the counterions may be dependent on the charge of the peptide and any additives. Thus, the counterions may be anions or cations. In general, the counterions may be cytocompatible. In certain embodiments, the counterions may be biocompatible. For instance, the counterions may comprise acetate, citrate, ammonium, fluoride, or chloride. In other embodiments, the preparation or peptide may be substantially free of chloride counterions.

In exemplary embodiments, the preparation or peptide may comprise an effective amount of acetate counterions. In particular, preparations having a peptide concentration which comprises residual TFA may have an amount of acetate counterions sufficient to balance the residual TFA. Briefly, TFA is commonly used to release synthesized peptides from solid-phase resins. TFA is also commonly used during reversed-phase HPLC purification of peptides. However, residual TFA or fluoride may be toxic and undesirable in peptides intended for clinical use. Furthermore, TFA may interact with the free amine group at the N-terminus and side chains of positively charged residues (for example, lysine, histidine, and arginine). The presence of TFA-salt counterions in the peptide preparation may be detrimental for biological material and may negatively affect the accuracy and reproducibility of the intended peptide activity.

TFA-acetate salt exchange by acetate or hydrochloride may be employed to counteract some or all of the negative effects of TFA described above. The inventors have determined the acetate counterion is surprisingly well suited for maintaining biological activity of the peptide preparation and for controlling solubility of the peptide and charge for self-assembly of the peptide. Furthermore, acetic acid (pKa=4.5) is weaker than both trifluoroacetic acid (pKa about 0) and hydrochloric acid (pKa=−7). Acetate counterions may additionally control pH of the peptide preparations to be physiologically neutral.

The preparation may have variable hydrogelation kinetics. In accordance with certain embodiments, the hydrogelation kinetics of the preparation may be designed for a particular mode of administration. The preparation may be administered as a liquid. The preparation may be administered as a solid or semi-solid. The preparation may be administered as a gel. The preparation may be administered as a combination of hydrogel suspended in a liquid. The preparation may have a variable apparent viscosity. For instance, the preparation may have an apparent viscosity effective to allow injection under the conditions of administration. In certain embodiments, the preparation may have an apparent viscosity which decreases with increasing shear stress.

The preparation may be configured to reversibly self-assemble and disassemble in response to applied stress, for example, applied mechanical force. The solid or gel preparation may become disrupted with increasing applied stress, to be later restored once the applied stress is reduced. The solid or gel may become fluid in response to applied stress, for example, during delivery through a delivery device. The peptide may be capable of undergoing sequential phase transitions in response to applied stress. The peptide may be capable of recovering after each one or more sequential phase transitions.

The preparation may be configured to reversibly self-assemble and disassemble responsive to at least one of change in temperature, change in pH, contact with an ion chelator, dilution with a solvent, applied sound wave, lyophilization, vacuum drying, and air drying. The administered fluid may conform to tissue voids before reforming as a solid or gel. Thus, the solid or gel preparation may be injectable, flowable, or sprayable under the appropriate shear stress. Once administered, the preparation may be restored to a solid or gel form, substantially conforming to the target site. The formation may occur within less than a minute, about one minute, less than about 2 minutes, less than about 3 minutes, less than about 5 minutes, or less than about 10 minutes. The formation may occur within about one minute, less than about 30 seconds, less about 10 seconds, or about 3 to 5 seconds.

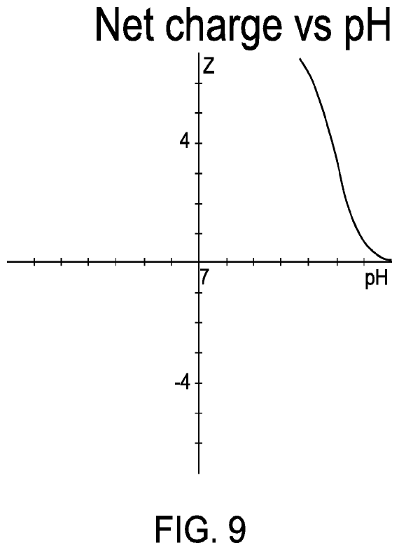

The peptide may be purified. For example, the peptide may be lyophilized. As shown in FIG. 9, net charge may be quantified as a function of pH value. The exemplary peptide measured in FIG. 9 is an arginine-rich peptide having two lysine residues. The exemplary peptide of FIG. 9 has a net charge of +9 at a pH of 7. Other peptides are within the scope of the disclosure. For example, the purified peptide may have a net charge between −9 to +11 at pH 7, for example, −7 to +9 at pH 7. As disclosed herein, “net charge” may refer to a total electric charge of the peptide as a biophysical and biochemical property, typically as measured at a pH of 7.

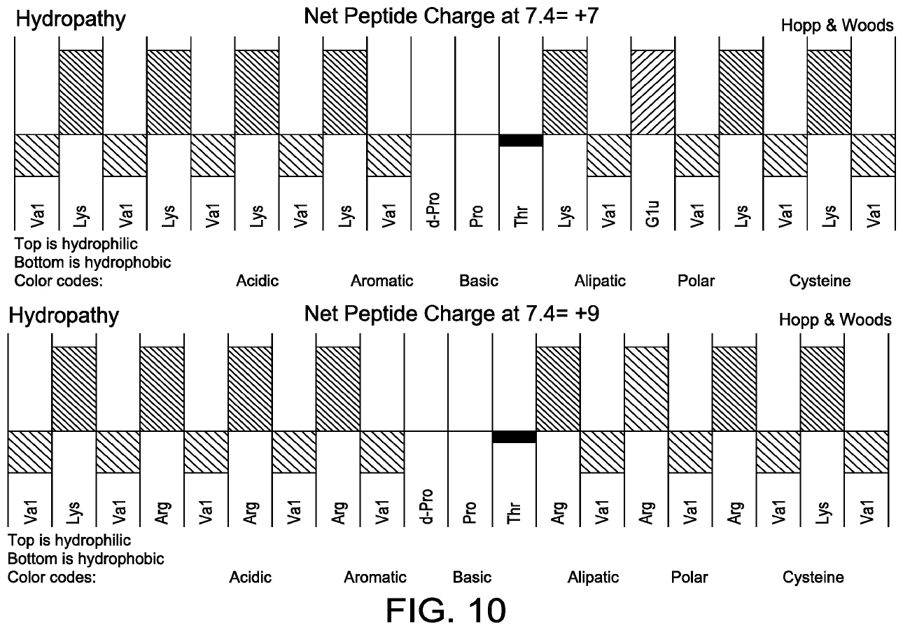

The purified peptide may have a net charge of from −7 to +11 at pH 7. In some embodiments, the peptide may have a net charge of from +2 to +9, for example, +5 to +9 or +7 to +9. The purified peptide may have a charge of about +5, +6, +7, +8, +9, +10, or +11 at pH 7. Exemplary peptides having a charge of +5 to +9 include VLTKVKTKV(d)PPTKVEVKVLV, VKVRVRVRV(d)PPTRVRVRVKV, and VKVRVRVRV(d)PPTRVEVRVKV. In other embodiments, the purified peptide may be substantially neutral. In other embodiments, the peptide may have a net negative charge. An exemplary peptide having a net negative charge is VEVSVSVEV(d)PPTEVSVEVEV. As shown in FIG. 10, a single substitution of glutamic acid in the peptide sequence may alter net peptide charge from +7 (top panel) to +9 (bottom panel) at pH 7, as well as alter isoelectric point from 11.45 to 14. Net charge may be selected by peptide design. Design of electrostatic charge in the peptide hydrogel may allow control of charge interaction with cell membrane and proteins.

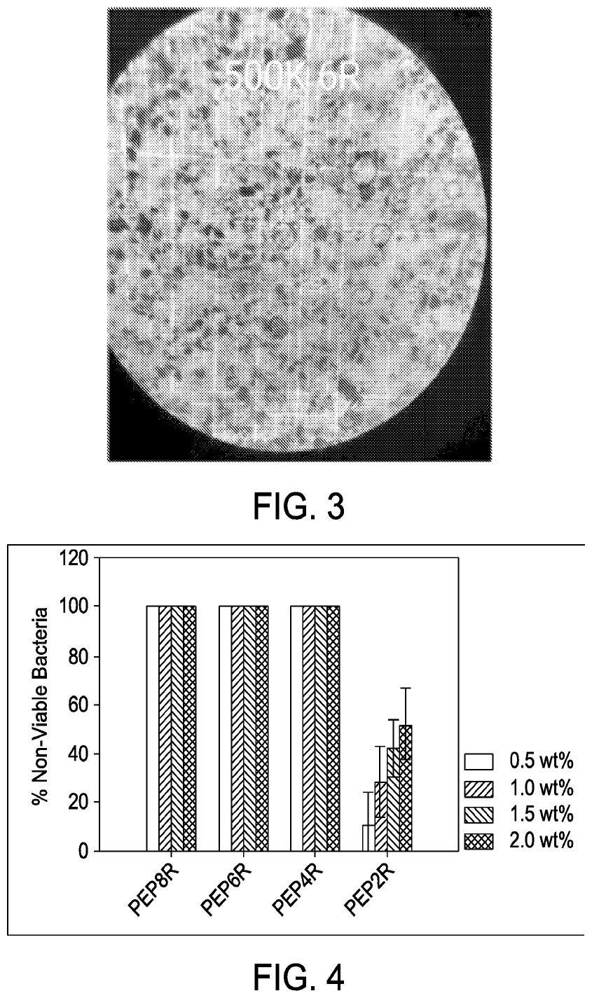

The peptide may be designed to have a charge that adsorbs and/or promotes deactivation of proteins at a target site of administration. For instance, positively charged peptide hydrogels may promote adsorption of negatively and neutrally charged molecules such as small molecules, proteins, and extravesicular membranes. Negatively charged peptide hydrogels may promote adsorption of positively and neutrally charged molecules such as small molecules, proteins, and extravesicular membranes. Furthermore, the peptide may be designed to have regions of positive, neutral, or negative charge, to varying degrees. In certain embodiments, the peptide charge may be designed such that when placed into a rich solution of charged molecules, the peptide may soak out or absorb the molecules into the hydrogels attaching the molecules to the peptides by adsorption. FIG. 3 is a microscopy image showing negatively charged Trypan blue adsorbed on a positively charged hydrogel.

The purified peptide may have greater than 70% w/v, greater than 80% w/v, or greater than 90% w/v nitrogen, for example, between 70% w/v and 99.9% w/v nitrogen.

The purified peptide may have a bacterial endotoxin level of less than about 10 EU/mg, for example, less than about 5 EU/mg, less than about 2 EU/mg, or less than about 1 EU/mg. In other embodiments, the purified peptide may have an endotoxin level of between about −0.010 to −0.015 EU/ml. For instance, the purified peptide may have an OD at 410 nm of between 0.004 to 0.008, for example, about 0.006. The peptide hydrogel may have an OD at 410 nm of between 0.010 to 0.020, for example, about 0.015. In some embodiments, the purified peptide and/or preparation may be substantially free of endotoxins.

The purified peptide in the biocompatible solution may have a water content of between about 1% w/v and about 20% w/v, for example, at least about 10% w/v, or less than about 15% w/v.

The purified peptide may have an isoelectric point of between about 7-14. For example, the purified peptide may have an isoelectric point of about 7, 8, 9, 10, 11, 12, 13, or 14.

The purified peptide may be configured to self-assemble into a hydrogel having a shear modulus of between about 2 Pa to 3500 Pa as determined by rheology testing. For example, the purified peptide may self-assemble into a hydrogel having a shear modulus of greater than 100 Pa, between 100 Pa and 3500 Pa, between 100 Pa and 3000 Pa, between 2 Pa and 1000 Pa, or between 2 Pa and 500 Pa. For example, a formulation having 0.75% w/v peptide may have a shear modulus of between about 2 Pa and 500 Pa. A formulation having 1.5% w/v peptide may have a shear modulus of between about 100 Pa and 3000 Pa. A formulation having 3.0% w/v peptide may have a shear modulus of between about 1000 Pa and 10000 Pa. Thus, shear modulus of the hydrogel may be controlled by selection of peptide concentration in the formulation.

The peptide may be designed to adopt a predetermined secondary structure. For example, the peptide may be designed to adopt a β-hairpin secondary structure, as previously described. The predetermined secondary structure may comprise a structure preselected from at least one of a β-sheet, an α-helix, and a random coil. In exemplary embodiments, the hydrophobic amino acid residues (for example, quantity, placement, and/or structure of the hydrophobic amino acid residues) may be selected to self-assemble the peptide into a polymer having a majority of β-sheet structures. In particular embodiments, the hydrophobic amino acid residues may be selected to control stiffness of the hydrogel. For example, an amount and type of hydrophobic amino acid residues may be selected to control stiffness of the hydrogel.

In some embodiments, an external stimulus such as temperature, change in pH, light, and applied sound wave may be used to control and promote preferential secondary structure formation of the self-assembling peptide. Control of the secondary structure formation may enhance biological, biophysical, and chemical therapeutic functions of the peptide. For example, higher cell membrane penetration of self-assembling peptides may be achieved by exposing β-hairpin peptides to high pH (for example, at least pH 9) or high temperatures (for example, at least 125° C.) or low temperatures (for example, 4° C. or lower). The result is hydrogels with a peptide secondary structure having a majority β-sheet or α-helix formation.

The peptide may be designed to give the preparation shear-thinning properties. In particular, the peptide may be designed to be injectable. For instance, the peptide may be designed to be an injectable solid or gel by employing shear-thinning kinetics. The preparation, in the form of a solid or gel prior to application, may be configured to shear-thin to a flowable state under an effective shear stress applied during administration by the delivery device. In some exemplary embodiments, the solid or gel may shear-thin to a flowable state during injection or topical application with a syringe. Other modes of administration may be employed. The solid or gel may shear-thin to a flowable state during endoscopic administration. The solid or gel may be configured to shear-thin to flow through an anatomical lumen, for example, an artery, vein, gastrointestinal tract, bronchus, renal tube, genital tract, etc. In some embodiments, the shear thinning properties may be employed during transluminal procedures. The peptide may be designed to be sprayable. For example, the peptide may be designed for administration as a spray or other liquid droplet, for example, other propelled liquid droplet, by employing shear-thinning kinetics, as previously described.

The shear-thinning kinetics of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions. In particular, shear-thinning may be controlled by altering the peptide purity to achieve the desired shear-thinning kinetics. The net charge of the peptide may be positive. The net charge of the peptide may be negative.

Shear-thinning may be induced by mechanical agitation to the hydrogel or environmental stimulus. Mechanical agitation may be induced, for example, through delivery or sonication mixing. Environmental stimulus may be induced by addition of heat, light, ionic agents, chelator agents, buffers, or proteins, or altering pH level.

Thus, the preparations may be substantially flowable. The methods may include dispensing the preparation through a cannula or needle. The methods may include conformally filling wound beds of any size and shape. The peptide hydrogels may have shear-thinning mechanical properties. The shear-thinning mechanical properties may allow the gel network to be disrupted and become a fluid during administration, for example, during injection from a needle or administration with a spray nozzle. When the applied stress ceases, the gel network may reform and the elastic modulus may be restored within a predetermined period of time, for example, several minutes. The shear-thinning peptide hydrogels may be employed to protect cells from damage during injection, showing an improved viability over direct injection in saline or media. The shear-thinning hydrogel may display non-Newtonian fluid flow, which may allow for effective mixing of excipients, for example, within minutes to a couple hours. In some embodiments, dyes, small molecules, and large molecules may be substantially homogeneously dispersed within the hydrogel in less than 120 minutes, for example, between 30-120 minutes.

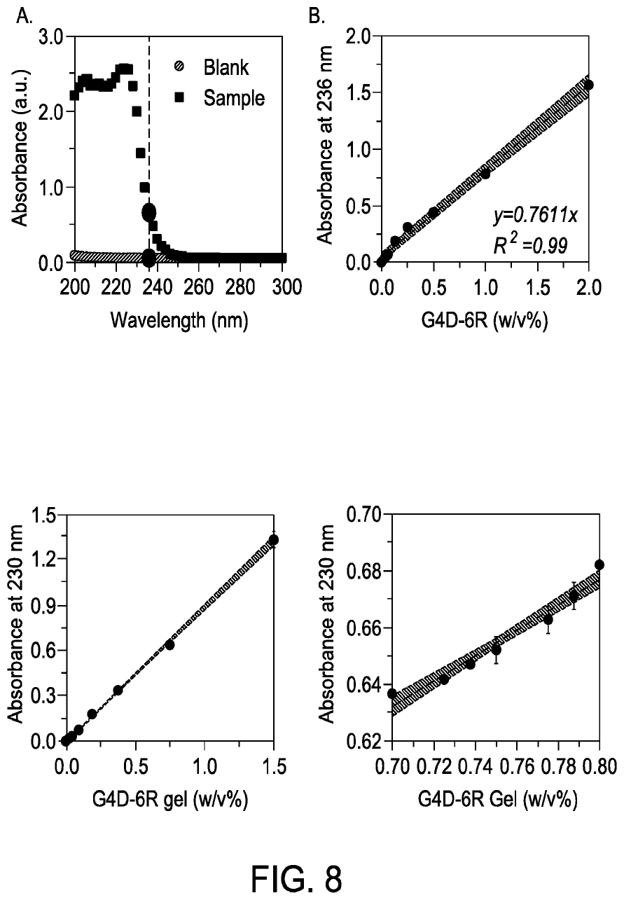

The peptide may self-assemble into a translucent hydrogel. In some embodiments, the peptide may self-assemble into a substantially transparent hydrogel. The transparency of the hydrogel may enable a user or healthcare provider to view surrounding tissues through the hydrogel. In exemplary embodiments, a surgical procedure may be performed without substantial obstruction of view by the hydrogel. The hydrogel may have at least about 70%, at least about 80%, at least about 90%, at least about 95%, at least about 99%, or about 100% light transmittance. The hydrogel may be colorless. The light transmittance and color of the hydrogel may be engineered by tuning the sequence of the peptide and/or the composition of the preparation or solution. As shown in the graphs of FIG. 8, transparency of the peptide hydrogels may be quantified by absorbance measurements. The exemplary peptide hydrogels measured in FIG. 8 are substantially transparent.

In some embodiments, the preparation may include a dye. The dye may be a food-grade dye or a pharmaceutical-grade dye. The dye may be cytocompatible. The dye may be biocompatible. In general, the dye may assist the user or healthcare provider to view the hydrogel after application. The preparation may include an effective amount of the dye to provide a desired opacity of the hydrogel. The hydrogel may comprise an effective amount of the dye to have a light transmittance of less than about 60%, less than about 50%, less than about 40%, less than about 30%, less than about 20%, or less than about 10%. The hydrogel may be substantially opaque when including the dye.

The peptide may self-assemble into a substantially ionically-crosslinked hydrogel. “Ionic crosslinkage” may refer to ionic bonds between peptides to form secondary structure proteins and/or between secondary structure proteins that form the hydrogel tertiary structure. The shear-thinning properties of the hydrogel may be enabled by physical crosslinks, allowing ionic bonds to be broken and reformed. In accordance with certain embodiments, the hydrogel is formed of a majority of ionic crosslinks. For example, more than 50%, more than 60%, more than 70%, more than 80%, more than 90%, more than 95%, more than 99%, or substantially all of the physical crosslinks of the formed hydrogel may be ionic in nature.

The preparation and/or assembled hydrogel may be designed to have a substantially physiological pH level. The preparation or hydrogel may have a pH level of between about 4.0 and 9.0. In some embodiments, the preparation or hydrogel may have a pH level of between about 7.0 and 8.0. The preparation or hydrogel may have a pH level of between about 7.3 and 7.5. The substantially physiological pH may allow administration of the preparation at the time of gelation. In some embodiments, the hydrogel may be prepared at a point of care. The methods may comprise mixing the preparation with a buffer configured to induce self-assembly, optionally agitating the mixture, and administering the preparation or hydrogel at a point of care. The administration may be topical or parenteral, as described in more detail below.

The peptide may be designed to self-assemble in response to a stimulus. The stimulus may be an environmental stimulus, e.g., change in temperature (e.g., application of heat), exposure to light, change in pH, applied sound waves, or exposure to ionic agents, chelator agents, or proteins. The stimulus may be mechanical agitation, e.g., induced through delivery, sonication, or mixing. In some embodiments, the methods may comprise administering the preparation as a liquid. The methods may comprise administering the preparation as a gel. The methods may comprise administering the preparation as a solid or semi-solid.

In some embodiments, the preparation may be designed to self-assemble after a lapsed period of time. For example, the preparation may be designed such that the peptide is configured to begin self-assembly in less than about 5 minutes, in less than about 3 minutes, in less than about 2 minutes, in less than about 30 seconds, in less than about 10 seconds, or in less than about 3 seconds. In certain embodiments, the preparation may be designed such that the peptide is configured to self-assemble, i.e. be substantially self-assembled, within about 60 minutes, within about 30 minutes, within about 15 minutes, within about 10 minutes, within about 5 minutes, within about 3 minutes, within about 2 minutes, within about 30 seconds, within about seconds, within about 5 seconds, or within about 3 seconds. The preparation may have a composition configured to control timing of self-assembly. For example, the preparation may have a composition configured for timed release of ionic agents or pH-altering agents. In certain embodiments, the sequence or structure of the peptide may be designed to control self-assembly of the peptide.

In some embodiments, the methods may comprise combining the preparation with a buffer. The “buffer” may refer to an agent configured to induce gelation, prior to, subsequently to, or concurrently with administration of the preparation to the subject. Thus, in some embodiments, the preparation may comprise a buffer. For example, the preparation may comprise or be combined with up to about 1000 mM of the buffer. The buffer may comprise an effective amount of ionic salts and a buffering agent, for example, to induce gelation and/or provide desired properties. For example, the buffer may be formulated to control or maintain pH of the preparation.

In particular embodiments, the buffer may have an effective amount of ionic salts to control stiffness of the hydrogel. The “ionic salt” may refer to a compound which dissociates into ions in solution. The buffer may comprise between about 5 mM and 400 mM ionic salts. For example, the buffer may comprise between about 5 mM and 200 mM ionic salts, between about 50 mM and 400 mM ionic salts, between about 50 mM and 200 mM ionic salts, or between about 50 mM and 100 mM ionic salts. The ionic salt may be one that dissociates into at least one of sodium, potassium, calcium, magnesium, iron, ammonium, pyridium, quaternary ammonium, chloride, citrate, acetate, and sulfate ions. The ionic salts may comprise sodium chloride, ammonium chloride, magnesium chloride, potassium chloride, calcium chloride, ammonium sulfate, magnesium sulfate, sodium sulfate, potassium sulfate, calcium sulfate, sodium bicarbonate, and combinations thereof.

In exemplary embodiments, the buffer may comprise between about 1 mM and about 200 mM sodium chloride. For example, the buffer may comprise between about 10 mM and about 150 mM sodium chloride, for example between about 50 mM and about 100 mM sodium chloride.

The buffer may comprise counterions. The buffer may have an effective amount of counterions to render the hydrogel substantially electrically neutral. The buffer may have an effective amount of counterions to induce self-assembly of the peptide. The concentration of counterions may be dependent on the composition of the peptide preparation. Additionally, the charge of the counterions may be dependent on the charge of the peptide preparation. Thus, the counterions may be anions or cations. In general, the counterions may be cytocompatible. In certain embodiments, the counterions may be biocompatible. For instance, the counterions may comprise acetate or chloride. In other embodiments, the biocompatible solution may be substantially free of chloride counterions.

The buffer may comprise from about 1 mM to about 150 mM of a biological buffering agent. For example, the buffer may comprise from about 1 mM to about 100 mM of a biological buffering agent, from about 1 mM to about 40 mM of a biological buffering agent, or from about 10 mM to about 20 mM of a biological buffering agent. The biological buffering agent may be selected from Bis-tris propane (BTP), 4-(2-hydroxyethyl)-1-piperazineethanesulfonic acid (HEPES), Dulbecco's Modified Eagle Medium (DMEM), tris(hydroxymethyl)aminomethane (TRIS), 2-(N-Morpholino)ethanesulfonic acid hemisodium salt, 4-Morpholineethanesulfonic acid hemisodium salt (MES), 3-(N morpholino)propanesulfonic acid (MOPS), and 3-(N-morpholino)propanesulfonic acid (MOBS), Tricine, Bicine, (tris(hydroxymethyl)methylamino)propanesulfonic acid (TAPS), N-(2-Acetamido)-2-aminoethanesulfonic acid (ACES), β-Hydroxy-4-morpholinepropanesulfonic acid, 3-Morpholino-2-hydroxypropanesulfonic acid (MOPSO), (N,N-bis(2-hydroxyethyl)-2-aminoethanesulfonic acid) (BES) and combinations thereof. Other biological buffering agents are within the scope of the disclosure.

In exemplary embodiments, the buffer may comprise from about 1 mM to about 150 mM of BTP. The buffer may comprise from about 10 mM to about 100 mM BTP, for example, from about 10 mM to about 50 mM BTP, from about 10 mM to about 40 mM, from about 20 mM to about 40 mM, or from about 20 mM to about 40 mM.

The buffer may additionally comprise at least one of water, an acid, and a base. The acid and/or base may be added in an amount effective to control pH of the buffer to be a substantially physiological pH. In other embodiments, the buffer may be acidic, alkali, or substantially neutral. The buffer may be selected to control pH of the hydrogel and maintain a desired pH at the target site. For example, to control pH of the hydrogel to be a substantially physiological pH at the target site. Thus, the properties of the buffer may be selected based on the target site. The buffer may have additional properties as selected, for example, net charge, presence or absence of additional proteins, etc. The buffer may additionally comprise one or more minerals.

The preparation may further comprise an effective amount of a mineral clay. The preparation may comprise between about 0.1% w/v to about 20% w/v of the mineral clay. For example, the preparation may comprise 0.75% w/v, 1.5% w/v, 2% w/v, 3% w/v, 4% w/v, 8% w/v, 10% w/v, or 20% w/v of the mineral clay. The amount of the mineral clay may be effective to provide desired rheological properties for the target site of application. The amount of the mineral clay may be effective to form a film. The mineral clay may be natural or synthetic. The mineral clay may comprise at least one of laponite and montmorillonite. In some embodiments, the preparation may comprise from a 1:1 to 1:2 ratio (w/v) of the peptide to mineral clay. For example, the ratio of peptide to mineral clay in the preparation may be 1:1, 3:4, 3:8, or 1:2 (w/v).

The preparation may be formulated for a target indication. For instance, the preparation may be formulated for treatment of a microbial infection or inhibition of proliferation of a microorganism, such as a pathogenic microorganism. The preparation may be formulated for treatment of a fungal infection or inhibition of proliferation of a fungal organism. The preparation may be formulated for cell culture and/or cell delivery. The preparation may be formulated for treatment or inhibition of a wound, such as a chronic wound, or biofilm. The preparation may be formulated by engineering the peptide as described in more detail below. The preparation may be formulated by selecting the biocompatible solution and/or additives.

In certain embodiments, the preparation may be formulated for a combination treatment. The preparation may include at least one active agent configured to provide a combination treatment. In some embodiments, the preparation may exhibit synergistic results with combination of the active agent. The active agent may be, for example, an antibacterial composition, an antiviral composition, an antifungal composition, an anti-tumor composition, an anti-inflammatory composition, a hemostat, a cell culture media, a cell culture serum, an anti-odor composition, an analgesic, local anesthetic, or a pain-relief composition. The preparation may be formulated for administration in conjunction with one of the aforementioned compositions. The preparation may be formulated for simultaneous or concurrent combination administration. The preparation may be formulated for sequential combination administration.

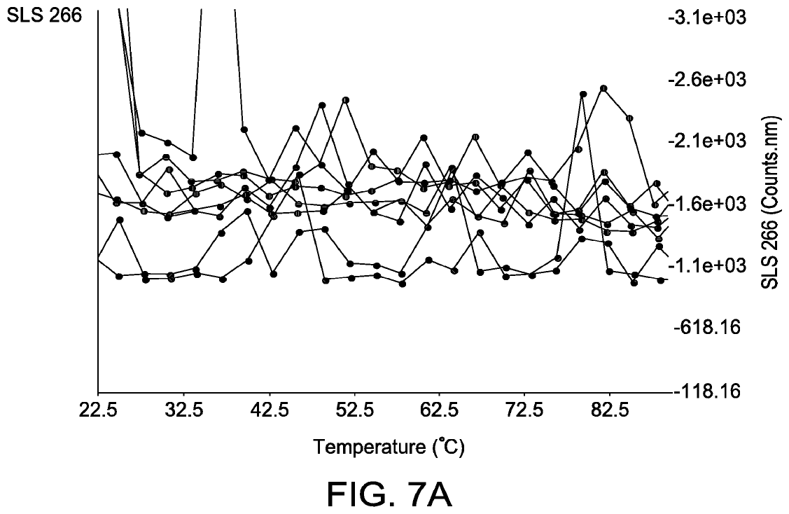

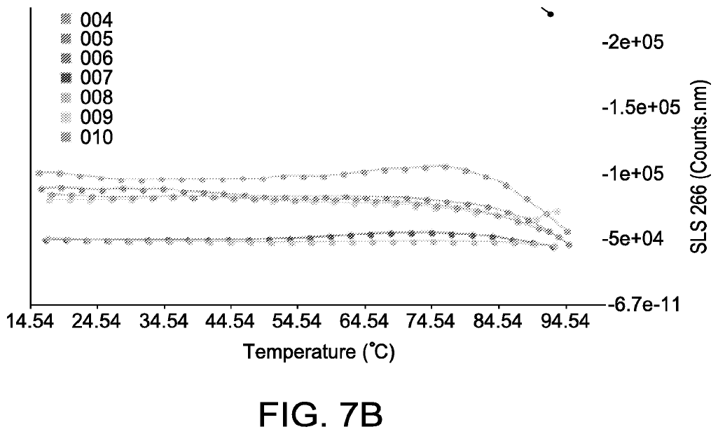

In some embodiments, the preparation and/or hydrogel may be designed to be thermally stable between −20° C. and 150° C., between −20° C. and 125° C., between −20° C. and 100° C., between 2° C. and 125° C., and between 37° C. and 125° C. As disclosed herein, “thermal stability” refers to the ability to withstand temperature treatment without substantial degradation, loss of biological activity, or loss of chemical activity. The graphs of FIGS. 7A-7B show peptide aggregation as measured by static light scattering (SLS) at 266 nm of exemplary peptides as a function of temperature. The exemplary peptides include arginine, lysine, valine, threonine, and proline residues. As shown in the graphs of FIGS. 7A-7B, the peptide hydrogels and peptides are thermostable as a function of temperature.

In certain embodiments, the preparation and/or peptide may be mechanically stable. For instance, the preparation may be shear thinned or sonicated. The preparation may be sonicated without substantial degradation, loss of biological or chemical activity. The preparation may be shear thinned without substantial degradation, loss of biological or chemical activity.

In certain embodiments, the preparation and/or peptide may be sterile or sterilized. The preparation and/or peptide may be sterilized by autoclave sterilization. During autoclave sterilization, the preparation and/or peptide may be heated to a temperature of between 120° C. to 150° C., for example, up to 125° C., up to 135° C., or up to 150° C. The preparation and/or peptide may be held at autoclave temperature for at least about 2 minutes, for example, between about 2-20 minutes or between about 10-160 minutes. The autoclave sterilization may be sufficient to sterilize at least about 90%, 95%, 99%, 99.9%, 99.99%, 99.999%, or 100% of any pathogenic microorganism. The preparation and/or peptide may remain stable during and after autoclave sterilization. For instance, the preparation and/or peptide may remain physically, chemically, biologically, and/or functionally stable after autoclave sterilization.

In certain embodiments, the preparation and/or peptide may be pasteurized. During pasteurization, the preparation and/or peptide may be heated to a temperature of between 50° C. to 100° C., for example, up to 60° C., up to 70° C., or up to 100° C. The preparation and/or peptide may be held at pasteurization temperature for at least about 15 seconds, for example, between about 1-30 minutes or between about 3-15 minutes. The pasteurization may be sufficient to sterilize at least about 90%, 95%, 99%, 99.9%, 99.99%, or 99.999% of any pathogenic microorganism.

In certain embodiments, the preparation may be sterilized by ultra-high temperature (UHT) or high temperature/short time (HTST) sterilization. During UHT or HTST sterilization, the preparation and/or peptide may be heated to a temperature of between 100° C. to 150° C., for example, up to 130° C., up to 140° C., or up to 150° C. The preparation and/or peptide may be held at UHT or HTST temperature for at least about 15 seconds, for example, between about less than 1 minute to about 6 minutes, for example, between about 2-4 minutes. The UHT or HTST sterilization may be sufficient to sterilize at least about 90%, 95%, 99%, 99.9%, 99.99%, or 99.999% of any pathogenic microorganism.

In certain embodiments, the sterilization or pasteurization may be terminal. Terminal sterilization or pasteurization may refer to treatment of the preparation in a sealed end-use package.

The preparation and/or peptide may be stable during and after heat treatment. As disclosed herein, stability during and after heat treatment, for example, autoclave sterilization, may refer to reduced or inhibited degradation, biological activity, and chemical activity. For instance, the preparation and/or peptide may be heat treated without degradation, a loss of biological activity, or a loss of chemical activity. Biological activity may refer to any bioactive property of the peptide disclosed herein. In some embodiments, biological activity may refer to antimicrobial activity. Chemical activity may refer to any chemical or physicochemical property of the peptide disclosed herein. In some embodiments, chemical activity may refer to the ability to self-assemble and or shear-thinning properties of the peptide disclosed herein. Thus, the preparation and/or peptide may be heat treated without loss of antimicrobial activity, self-assembly, or shear-thinning properties. In certain embodiments, heat treatment may enhance one or more biological activity or chemical activity of the peptide and/or preparation. For example, heat treatment may enhance antimicrobial activity, self-assembly, or shear-thinning properties of the peptide or preparation.

The preparation may be sterile. For example, the preparation may remain substantially sterile without the addition of a preservative. The preparation may be substantially sterile without gamma irradiation treatment. The preparation may be substantially sterile without electron beam treatment.

The preparation may have a predetermined shelf-life. “Shelf-life” may refer to the length of time for which the preparation may remain stable and/or maintain efficacy after storage under the given conditions. The preparation and/or hydrogel may have a shelf-life of at least about 1 year at a temperature between −20° C. and 150° C. For instance, the preparation and/or hydrogel may have a shelf-life of at least about 1 year at room temperature (between about 20° C. and 25° C.). The preparation and/or hydrogel may have a shelf-life of at least about 2 years, about 3 years, about 4 years, about 5 years, or about 6 years at room temperature. The preparation and/or hydrogel may be stable at a pressure of up to about 25 psi, for example, up to about 15 psi.

The peptide may be capable of self-assembly at a temperature between 2° C. and 40° C. For example, the peptide may be capable of self-assembly in an environment having a temperature between 2° C. and 20° C., between 20° C. and 25° C., or between 36° C. and 40° C.

The peptide may be substantially unassembled at temperatures higher than 40° C. For instance, the peptide preparation may be substantially liquid at temperatures between 40° C. and 150° C. The peptide preparation may be substantially liquid and thermally stable at temperatures between 40° C. and 125° C. or up to 150° C. Temperature may be controlled for handling of the preparation. For example, the preparation may be heated to a temperature greater than 40° C. for packaging, handling, and/or administration in a liquid state.

The preparation may be formulated for a desired route of administration. For example, the preparation may be formulated for topical or parenteral administration. In particular, the preparation may be engineered to have a viscosity appropriate for topical administration or parenteral administration. Preparations for topical administration may be formulated to withstand environmental and mechanical stressors at the site of administration or target site. Preparations for parenteral administration may be formulated to reduce migration from the site of administration or target site. In other embodiments, preparations for parenteral administration may be formulated to trigger migration from a site of administration to the target site. The preparation may be formulated for administration by a particular delivery device. For example, the preparation may be formulated for administration by spray, dropper, or syringe. The preparation may be formulated for administration by injection or catheter.

Table 1 includes the analytical characterization of three exemplary peptide preparation samples. The exemplary peptides have arginine-rich sequences comprising two lysine amino acid residues. The values were detected by conventional detection methods. Components indicated “N.D.” were below detection limit. Peptide purification, residual solvents, peptide content, and water content, may be selected to control antimicrobial activity and cell membrane disruption potential of the hydrogels.

TABLE 1
Exemplary Peptide Preparations

| | | | | | | Residual Solvents | | | | | | | | |
|---|---|---|---|---|---|---|---|---|---|---|---|---|---|---|
| | | | | | | | | | | N,N- | | | | |
| | | | | | Net | | | | Triethyl | Dimethyl | Ethyl | | | |
| | Purity | Acetate | TFA | Water | Peptide | Isopropanol | Methanol | Acetonitrile | amine | formamide | ether | | | |
| # | (%) | (%) | (%) | (%) | (%) | (ppm) | (ppm) | (ppm) | (ppm) | (ppm) | (ppm) | DCM | DIC | MPS |
| 1 | 97.9 | 19.3 | N.D. | 6.5 | 78.6 | 81 | 28 | 17 | N.D. | N.D. | N.D. | N.D. | N.D. | N.D. |
| 2 | 93.7 | 16.3 | N.D. | 7.1 | 79.3 | 63 | 28 | 18 | 56 | 116 | N.D. | N.D. | N.D. | N.D. |
| 3 | 96.0 | 16.9 | N.D. | 6.2 | 76.1 | N.D. | N.D. | 4.6 | 2.9 | N.D. | N.D. | N.D. | N.D. | N.D. |

The purified peptide and hydrogel may be substantially endotoxin free without addition of a preservative or sterilization, as shown in Table 2. Thus, in some embodiments, the peptide preparation may be substantially free of a preservative.

TABLE 2
Endotoxin Levels of Different Compositions

| | OD at 410 nm | Endotoxin level (EU/mL) |
|---|---|---|
| Endotoxin free water | −0.000333333 | −0.016148109 |
| RO water | 3.436666667 | 1.267607913 |
| Tap water | 3.463666667 | 1.2776927 |
| Buffer solution | −0.056 | −0.036940201 |
| Peptide | 0.006 | −0.013782542 |
| Peptide hydrogel | 0.015 | −0.010420946 |

### The Self-Assembling Peptide Hydrogel

The preparations disclosed herein may be provided to self-assemble into a hydrogel having preselected properties. The polymeric hydrogel may have a substantially physiological pH. In general, the polymeric hydrogel may have a pH of between 4.0 and 9.0, for example, between 7.0 and 8.0, between 7.2 and 7.8, or between 7.3 and 7.5.

The polymeric hydrogel may be substantially transparent. For example, the polymeric hydrogel may be substantially free of turbidity, for example, visible turbidity. Visible turbidity may be determined by macroscopic and microscopic optical imaging. The polymeric hydrogel may be substantially free of peptide aggregates (peptide clusters), for example, visible peptide aggregates. Visible peptide aggregates may be determined by static light scattering (SLS) and UV-VIS testing. “Transparency” may refer to the hydrogel's ability to pass visible light. The substantially transparent hydrogel may have UV-VIS light absorbance of between about 0.1 to 3.0±1.5 at a wavelength of between about 205 nm to about 300 nm.

The assembled polymeric hydrogel may have a nano-porous structure. The polymeric hydrogel may be hydrated or substantially saturated. In some embodiments, the hydrogel may have between 90% w/v and 99.9% w/v aqueous solution, for example, between 92% w/v and 99.9% w/v or between 94% w/v and 99.9% w/v. The nano-porous structure may be selected to be impermeable to a target microorganism. Thus, the hydrogel may be used to encapsulate a target microorganism or to maintain the target site free from the target microorganism. The nano-porous structure may be selected to allow gaseous exchange at the target site. The polymeric hydrogel may have a nano-porous structure having a pore size of between 1 nm and 1000 nm, as selected (e.g., based on a target microorganism, target cell, or desired functionality). The polymeric hydrogel may have a fibril width of between 1 nm and 100 nm, as selected.

The hydrogel may generally be cationic in nature. In other embodiments, the hydrogel may be anionic in nature. In yet other embodiments, the hydrogel may be blended to contain multi-domains of cationic and/or anionic components. The hydrogel may be designed to have a preselected charge. The self-assembling peptide hydrogel disclosed herein may be tunable to biological functionality that supports the viability and function of transplanted therapeutic cells, to exhibit shear-thinning mechanical properties that allow easy and rapid administration in an intra-operative setting, to exhibit antimicrobial properties to control wound bioburden, to exhibit antiviral properties to treat or inhibit viral infection, and/or to exhibit antifungal properties to treat or inhibit fungal infection.

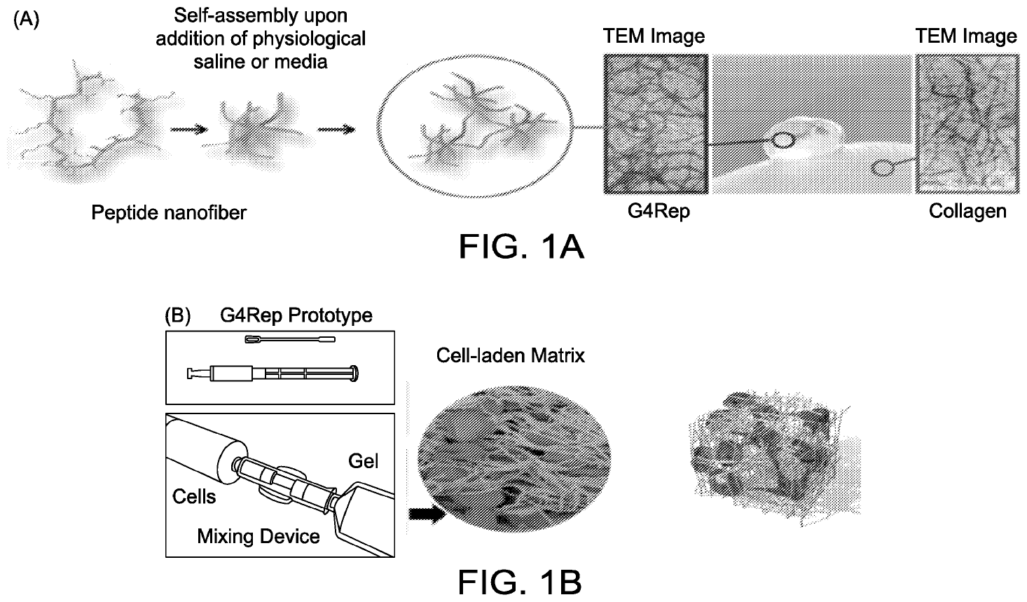

In particular, the peptide sequence and structure may include peptide functional groups that form nanofibers, which further self-assemble to form macromolecular structures (FIG. 1A-1B). The peptides may self-assemble in response to an environmental stimulus. The peptides may self-assemble in the presence of substantially physiological buffers, such as media or saline. The peptide hydrogels may assemble into an extracellular scaffolding matrix that is similar to native fibrillar collagen (FIG. 1A-1B). Schematics of gel matrix self-assembly and an exemplary nanostructure are shown in FIGS. 1A-1B. As shown in FIG. 1A, single peptide nanofibers self-assemble into a gel when ionic buffer is added. FIG. 1A includes a TEM image demonstrating that the nanostructure and pore size of the peptide gel look similar to native ECM (collagen). FIG. 1B includes a schematic drawing of an intra-operative mixing device for mixing a cell suspension with peptide gel matrix. A schematic SEM image in FIG. 1B of the cell-laden matrix demonstrates the exemplary concept of cells in matrix.

The peptide may be engineered by design to self-assemble into a hydrogel which is substantially biocompatible. The peptide may be engineered by design to self-assemble into a hydrogel that is cell friendly. In certain embodiments, the cell-friendly polymeric hydrogel may be non-inflammatory, and/or non-toxic. The cell-friendly polymeric hydrogel may be substantially biodegradable. The peptide may be engineered by design to be substantially antimicrobial, antiviral, and/or antifungal.

The short peptides and/or peptide functional groups may be produced synthetically. Thus, the peptides may provide ease of manufacturing, scale-up, and quality control. In general, the peptides may be manufactured without the use of plant or animal expression systems. The peptides may be substantially free of naturally occurring endotoxins and disease-transmitting pathogens. In addition, the peptide sequence and functional groups may be tuned, allowing a versatility in control and design of the assembled hydrogel, including with respect to physical and chemical properties, such as biodegradation, mechanical properties, and biological activity.

The peptide may have a functional group engineered for a target indication. For instance, the peptide may have a bioactive functional group. The target indication may be tissue engineering of a target tissue. The target indication may include, for example, cell culture, cell delivery, wound healing, and/or treatment of biofilm. Thus, the peptide may be engineered by design to self-assemble into a hydrogel which is substantially biocompatible. The peptide functional group may have between about 3 and about 30 amino acid residues. For example, the peptide functional group may have between about 3 and about 20 amino acid residues. The peptide functional group may have a sequence selected from RGD, IKVAV, YIGSR, LKKTETQ, SNKPGVL, PKPQQFFGLM, GKLTWQELYQLKYKGI, and GGG.

In some embodiments, the peptide may include a modification selected from a linker and a spacer. Peptide “linkers” may generally refer to short amino acid sequences included to link multiple domains of the peptide. Peptide “spacers” may generally refer to amino acid sequences positioned to link and control the spatial relationship of the multiple domains of the assembled protein. The linker or spacer may be hydrophobic or hydrophilic. The linker or spacer may be rigid or flexible. Exemplary spacers include aminohexanoic acid (Ahx) (hydrophobic) and poly (ethylene) glycol (PEG) (hydrophilic). Glycine rich spacers are generally flexible.

Exemplary bioactive functional groups include laminin, bone marrow homing, collagen (e.g., I, II, and VI), bone marrow purification, and RGD/fibronectin types. Exemplary bioactive functional groups include VEGF, Substance P, Thymosin Beta, Cardiac Homing Peptide, Bone Marrow Homing Peptide, Osteopontin, and Ostegenic peptide. Exemplary bioactive functional groups include those in Tables 3-5 below.

TABLE 3
Exemplary Bioactive Functional Groups

| ECM Motif | ECM Type |
|---|---|
| -GGPDSGR | Laminin |
| -GGSDPGYIGSR | Laminin |
| -GGSKPPGTSS | Bone marrow homing |
| -GGDGEA | Collagen II |
| -GGPFSSTKT | Bone marrow homing |
| -GGFLGFPT | Bone marrow purification |
| -GGRGDS | RGD/fibronectin |
| -GGIKVAV | Laminin |
| -GGFPDERGVEGPGP | Collagen I |
| -GGPRGDSGYRGDSF | Collagen VI |

TABLE 4
Exemplary Bioactive Functional Groups

| Protein/Peptide | Bioactive Motif |
|---|---|
| VEGF | GKLTWQELYQLKYKGI |
| Substance P | RPKPQQFFGLM |
| Thymosin Beta | SNKPG |
| Thymosin Beta | LKKTETQG |
| Cardiac Homing Peptide | CSTMLKAC |
| Bone Marrow Homing Peptide | GCPFSSTKTE |
| Osteopontin | TLEGTKKGHKLHLDY |
| Osteogenic Peptide | ALKRQGRTLYGFGG |

TABLE 5
Exemplary Bioactive Functional Groups

| Peptide name | Sequence | Source |
|---|---|---|
| RGD | XRGDX | Fibronectin, osteopontin, |
| | | BSP and others |
| pRGD/bsp-RGD | CGGNGEPRGDTYRAY | BSP |
| hBSP278-293 | YESENGEPRGDNYRAY | BSP |
| PHSRN | PHSRN | Fibronectin |
| F105 | YKRSRYT | FGF-2 |
| F119 | KRTGQYKLGSKTGPGQK | FGF-2 |
| F36 | PDGRVD | FGF-2 |
| F77 | KEDGRLL | FGF-2 |
| GFOGER | GGYGGGPC(GPP)₅GFOGER | Collagen type I |
| | (GPP)₅GPC | |
| DGEA | DGEA | Collagen type I |
| P15 | GTPGPQGIAGQRGVV | Collagen type I |
| AC-100 | ERGDNDISPFSGDGQ | MEPE |
| ODP (osteopontin-derived | DVDVPDGRGDSLAYG | Osteopontin |
| peptide) | | |
| RRETAWA | RRETAWA | Identified by phage display |
| YIGSR | YIGSR | Laminin |
| IKVAV | IKVAV | Laminin |
| Ln2-P3 | DLTIDDSYWYRI | Laminin |
| KRSR | KRSR | BSP, fibronectin, |
| | | osteopontin and others |
| Consensus heparin binding | FHRRIKA | BSP |
| domain | | |
| HBD peptide | RKKNPNCRRH | BMP-4 |
| BMP-2-derived peptide | NSVNSKIPKACCVPTELSAI | BMP-2 |
| BMP-2-derived peptide | KIPKASSVPTELSAISTLYL | BMP-2 |
| P24 | S^{[PO}₄^{]}KIPKASSVPTELSAIST | BMP-2 |
| | LYLDDD | |
| pBMP-9/SpBMP-9 | CGGKVGKA(CC/SS)VPTKL | BMP-9 |
| | SPISVLYK | |
| BMP-9-derived peptide | RKVGKASSVPTKLSPISILYK | BMP-9 |
| BMP-7-derived peptide | RTVPKPSSAPTQLNAISTLYF | BMP-7 |
| BFP-1 | GQGFSYPYKAVFSTQ | BMP-7 |
| OPD (osteopromotive- | DWIVA | BMP-2 |
| domain) | | |
| CBM | GLRSKSKKFRRPDIQYPDA | Osteopontin |
| | TDEDITSHM | |
| BCSP ™-1 | NGLPGPIGP | Collagen type I |
| OGP (osteogenic growth | ALKRQGRTLYGFGG | Naturally occurring |
| peptide) | | |
| OGP₁₀₋₁₄ | YGFGG | OGP |
| PTH₁₋₃₄ | SVSEIQLMHNLGKHLNSME | Parathyroid hormone |
| | RVEWLRKKLQDVHNF | |
| CB | NGVFKYRRRYYLYKHAYF | BSP |
| | YPHLKRFPVQ | |
| CTC peptide | IAGVGGEKSGGF | Collagen-IIIa |
| Substance-P | RPKPQQFFGLM | Naturally occurring |
| Endothelin-1 | CSCSSLMDKECVYFCHLD | Naturally occurring |
| | IIW | |
| QK | KLTWQELYQLKYKGI | VEGF |
| PBA2-1c | (CVRKIEIVRKK)₂-Ahx-Ahx- | PDGF |
| | Ahx-RKRKLERIAR | |
| Exendin 4 | HGEGTFTSDLSKQMEEEAVR | Glucagon-like peptide |
| | LFIEWLKNGGPSSGAPPPS | |
| OPD (osteopontin-derived | SVVYGLR | Osteopontin |
| peptide) | | |
| SPARC 113 | TLEGTKKGHKLHLDY | Osteonectin |
| SPARC 118 | KKGHK | Osteonectin |
| TP508 | AGYKPDEGKRGDACEGDS | Thrombin |
| | GGPFV | |
| RoY | YPHIDSLGHWRR | Designed |
| LL-37 | LLGDFFRKSKEKIGKEFKRI | hCAP18 |
| | VQRIKDFLRNLVPRTES | |

The peptide may have a functional group engineered to control or alter charge or pH of the peptide or preparation. A pre-selected charge or pH may provide bioactive properties. In some embodiments, a pre-selected charge or pH may provide antimicrobial, antifungal, and/or antiviral properties. In some embodiments, a pre-selected charge or pH may allow the preparation to be administered to a compatible target site.

The peptide may have an antimicrobial functional group. The antimicrobial functional group may include a conserved sequence of antimicrobial residues. In other embodiments, the antimicrobial functional group may overlap or partially overlap with the self-assembling functional group. In at least one embodiment, the peptide may have alternating or substantially alternating antimicrobial and self-assembling residues.

The peptide may have an antifungal functional group. The antifungal functional group may include a conserved sequence of antifungal residues. In other embodiments, the antifungal functional group may overlap or partially overlap with the self-assembling functional group. In at least one embodiment, the peptide may have alternating or substantially alternating antifungal and self-assembling residues.

The peptide may have an antiviral functional group. The antiviral functional group may include a conserved sequence of antiviral residues. In other embodiments, the antiviral functional group may overlap or partially overlap with the self-assembling functional group. In at least one embodiment, the peptide may have alternating or substantially alternating antiviral and self-assembling residues.

The self-assembled hydrogel may be designed to have cell protective properties at the target site. In particular, the self-assembled hydrogel may be designed to be protective against foreign microorganisms, e.g., pathogenic microorganisms. The self-assembled hydrogel may be designed to be protective against fungal organisms. The self-assembled hydrogel may be designed to be protective against immune attack from environmental immune cells. The antimicrobial, antiviral, antifungal, and/or protective properties of the hydrogel may not substantially affect the viability, growth, or function of cells at the target site.

The protective properties of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions. The peptide may be engineered to have positively charged, negatively charged, hydrophobic, or hydrophilic amino acid residues. In an exemplary embodiment, the peptide may provide antimicrobial, antiviral, and/or antifungal properties by inclusion amino acids which are positively charged at a substantially neutral pH level. Such amino acids may include, for example, arginine, lysine, tryptophan, and histidine.

The peptide hydrogel may additionally exhibit antimicrobial properties. In general, the antimicrobial properties may be provided by including an antimicrobial functional group. In some embodiments, the antimicrobial functional group may include a cation-rich peptide sequence. In exemplary embodiments, the antimicrobial functional group may include varying ratios of lysine (K) and arginine (R) (FIG. 4). The antimicrobial peptide hydrogel may provide antimicrobial effects against gram-positive and negative bacteria, including, for example, E. coli (FIG. 4), S. aureus, and P. aeruginosa. FIG. 4 is a graph showing antimicrobial activity (as percent non-viable E. coli remaining after 24 hours of administration) of varying concentrations of peptides having 8 arginine residues (PEP8R), 6 arginine residues (PEP6R), 4 arginine residues (PEP4R), and 2 arginine residues (PEP2R).

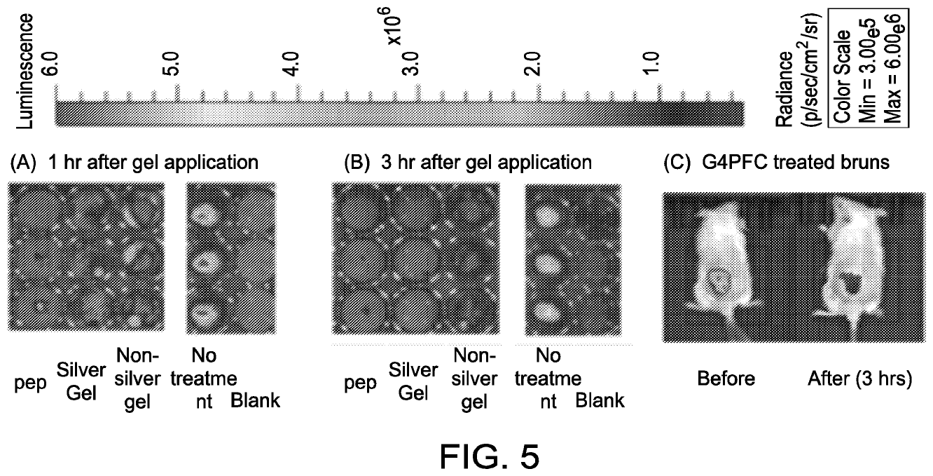

The peptide hydrogels may exhibit broad spectrum antimicrobial activity. In accordance with certain embodiments, the peptide hydrogels may reduce bioburden in vivo in partial thickness wounds inoculated with methicillin-resistant S. aureus (MRSA) (FIG. 5). FIG. 5 shows preliminary data demonstrating antimicrobial benefits of treating bioluminescent MRSA (US300) with peptide gels. Images (A) and (B) show wells plated with 100 μl of gel and 100 μl of US300 (1×10⁸ CFU/ml) demonstrating the antimicrobial activity of peptide gels compared to controls at 1 hour and 3 hours (n=3). Image (C) shows mice with partial thickness burns inoculated with 50 μl of 10⁸ CFU/ml US300 and treated with peptide gels. As shown in image (C), the mice exhibit reduced bioburden at 3 hours after administration.

In particular, the peptide hydrogel may exhibit antimicrobial properties against foreign and/or pathogenic microorganisms, and be compatible with the administered cells. For example, such peptide hydrogels may be compatible with mammalian erythrocytes and macrophages. In one exemplary experiment, when bacteria and mammalian cells were seeded simultaneously onto the peptide hydrogels disclosed herein, the bacteria were killed while the mammalian cells remained >90% viable after 24 hours and could continue to proliferate.

In some embodiments, the peptides may include functional groups to enhance or promote biological activity compatible or synergizing with MSC function. For example, in certain embodiments, the peptide sequence may contain a functional group that mimics fibronectin and promotes adhesion and proliferation of human MSCs to a greater extent than other ECM ligands. In certain embodiments, the peptide sequence may contain a functional group comprising a neuropeptide to promote diabetic wound healing by suppressing inflammation and inducing angiogenesis. In certain embodiments, the peptide sequence may contain a functional group comprising a neuropeptide to induce the proliferation and migration of MSCs, as well as enhance the immunomodulatory function of MSCs. In certain embodiments, the peptide sequence may contain a functional group to improve wound healing by increasing angiogenesis and inducing MSC proliferation and migration. In certain embodiments, the peptide sequence may lack a functional group that inhibits proteolytic activity. The peptide may be engineered to contain other functional groups known to one of skill in the art.

In vitro, the peptide hydrogels disclosed herein may allow cell invasion and proliferation in 3D constructs, allowing the hydrogels to serve as scaffolding matrices for tissue regeneration. The peptide hydrogels may show biocompatibility following subcutaneous implantation. Experiments show minimal cell debris or dead cells at the gel implantation site 7 days post-subcutaneous administration. Experiments further show minimal increases in cytokine concentration in the gel and surrounding tissues compared to naïve tissues, suggesting the gel has insignificant acute inflammation effects.

### Films Formed of the Self-Assembling Peptide Hydrogel

The self-assembling peptide preparations disclosed herein may be capable of crosslinking to form a film. It has been discovered that polymer formulations may be converted into therapeutic formulations, e.g., therapeutic polymers, by integrating self-assembling peptides. Furthermore, the properties of the self-assembling peptides disclosed herein may be selected to form therapeutic films targeted for a wide variety of uses.

The films may be formed by crosslinking the self-assembling peptide disclosed herein with a biocompatible polymer. The polymer may be a hydrophilic polymer. The polymer may be a hydrophobic polymer. The crosslinking may be chemical, e.g., covalent or ionic, enzymatic, or physical crosslinking. The films disclosed herein may be formulated to provide good shape retention and handleability properties and retain functional properties of the self-assembling peptide, such as antimicrobial efficacy and/or biocompatibility. The properties of the films may be controlled by designing the self-assembling peptide, polymer, and buffer of the formulation.

In some embodiments, the self-assembling peptide which forms the film may comprise at least one functional group available for crosslinking, e.g., covalent crosslinking, ionic crosslinking, or physical crosslinking. The at least one functional group available for crosslinking may comprise an amine, carboxyl, thiol, succinimidyl ester, maleimide, alkene, transcyclooctene, alkyne, ring-strained alkyne, dibenzylcyclooctyne, tetrazine, azide, or combinations thereof.

The self-assembling peptide may be modified at one or both of the N-terminus and the C-terminus. In some embodiments, the modification is an amidation. One exemplary amidation is functionalization with a cysteine moiety. Thus, the self-assembling peptide may be modified with a cysteine moiety at one or both of the N-terminus and the C-terminus for crosslinking. The cysteine moiety may form a disulfide bond with the hydrophilic polymer to increase mechanical stability of the hydrogel.

The self-assembling peptide may be combined with an agent to prevent pre-formation of cross-linking prior to the desired time. In some embodiments, the agent may be selected to prevent pre-formation of disulfide crosslinking prior to disulfide bond formation. The agent may be a reducing agent, e.g., a strong reducing agent. One exemplary reducing agent is Tris (2-carboxyethyl) phosphine (TCEP). Other agents are within the scope of the disclosure. Cross-linking may be initiated later by adjusting pH and/or temperature. Thus, crosslinking may be controlled by the addition of preventative agents and crosslinking agents in accordance with the desired use.

In some embodiments, the polymer which forms the film may comprise at least one functional group available for crosslinking, e.g., covalent crosslinking, ionic crosslinking, or physical crosslinking. The polymer may comprise at least two functional groups available for crosslinking. The one or more, for example, two or more, functional groups may be independently selected from an amine, carboxyl, hydroxyl, thiol, succinimidyl ester, alkene, transcyclooctene, alkyne, ring-strained alkyne, dibenzylcyclooctyne, tetrazine, azide, maleimide, and combinations thereof.

The polymer may be selected to provide a controllable mechanical structure to the film. For instance, the composition and/or amount of the polymer in the film may be selected to provide a desired mechanical structure to the film. The film may comprise from about 0.5% by weight to about 10% by weight of the polymer, for example, from about 1.0% by weight to about 5% by weight of the polymer. In some embodiments, the polymer has a molecular weight of less than about 50 kDa, for example, less than about 45 kDa, less than about 40 kDa, less than about kDa, or less than about 20 kDa.

The polymer may at least partially biodegradable. The polymer may be at least partially non-biodegradable.

In some embodiments, the polymer may be modified with succinimidyl carbonate (SC). One exemplary biodegradable film is formed with succinimidyl carbonate modified 4-arm PEG (PEG4SC). Such a film may be formed as a gel, cryogel, dehydrated gel, or dehydrated cryogel. Other polymers may be formed into biodegradable films by SC modification.

In some embodiments, the polymer may be modified with NH₂. One exemplary non-biodegradable film is formed with NH₂ modified 4-arm PEG (PEG4NH₂). Such a film may be formed as a gel, cryogel, dehydrated gel, or dehydrated cryogel. Other polymers may be formed into non-biodegradable films by NH₂ modification.

The polymer may comprise a homobifunctional linear polymer, a heterobifunctional linear polymer, a homofunctional branched polymer, a heterfunctional branched polymer, a homofunctional star polymer, a heterfunctional star polymer, a homofunctional dendritic polymer, a heterofunctional dendritic polymer, a copolymer, a random copolymer, a block copolymer, a diblock compolymer, a triblock copolymer, or combinations thereof. For instance, the polymer may be selected from a polyethylene glycol (PEG), derivative thereof or peptide conjugate thereof, polyethylene glycol-poly(lactide-co-glycolide) copolymer (PEG-PLGA), poly(ethylene glycol)-co-poly(glycolic acid) copolymer (PEG-co-PGA), polyvinyl alcohol (PVA), derivative thereof or peptide conjugate thereof, poly(2-hydroxyethyl methacrylate) (PHEMA), poly(N-isopropylacrylamide) (PNIPAAm), poly(acrylic acid) (PAAc), polyurethane, poloxamer, pluronics, polyurethane, polysaccharide, cellulose, carboxymethylcellulose, dextran, oxidized dextran, alginate, oxidized alginate, hyaluronic acid, chitosan, gelatin, elastin, collagen, carob gum, pullulan, and combinations thereof.

The film may be formed by crosslinking the self-assembling peptide with the polymer by a chemical crosslinker molecule or a coupling chemical agent. The crosslinker molecule or coupling chemical agent may be included with the self-assembling peptide preparation, polymer preparation, buffer, or provided separately.

The chemical crosslinking agent may comprise an amide bond forming agent, a carbodiimide activation agent, a click chemistry agent, a copper-free click chemistry agent, a Michael-type addition agent, a Schiff base reaction agent, or combinations thereof. The chemical crosslinking agent may be a photo-crosslinker. Thus, in some embodiments, crosslinking may be activated by light applied at an effective wavelength and intensity to induce crosslinking.

One exemplary crosslinker molecule is sulfosuccinimidyl 4-(N-maleimidomethyl) cyclohexane-1-carboxylate (Sulfo-SMCC). Sulfo-SMCC is a heterobifunctional crosslinker containing NHS ester and maleimide groups. Sulfo-SMCC may be used to react with sulfhydryl groups and amine groups on the peptide, e.g., a cysteine moiety containing self-assembling peptide.

The chemical crosslinker molecule may be an extract of red cabbage. One exemplary chemical crosslinker molecule is glutaraldehyde. Glutaraldehyde is a Schiff base reaction agent. Glutaraldehyde may be used to crosslink carbonyls (e.g., aldehydes and ketones) with groups, such as primary amines, hydroxylamine, etc.

The chemical crosslinker molecule may be genipin. Genipin is a chemical compound found in Genipa americana fruit extract. Genipin is an aglycone derived from an iridoid glycoside. Genipin may be used as a crosslinker for proteins, collagen, gelatin, chitosan, among others.

Other chemical crosslinker molecules are within the scope of the disclosure. The film may have a concentration of the chemical crosslinker molecule, e.g., glutaraldehyde or genipin, effective to control gelation kinetics of the film. For instance, in some embodiments, the film may comprise from about 0.1% by volume to about 2% by volume of the chemical crosslinker molecule, e.g., glutaraldehyde or genipin, for example, from about 0.5% by volume to about 1.0% by volume of the chemical crosslinker molecule, e.g., glutaraldehyde or genipin. In one exemplary embodiment, the film may have a concentration of glutaraldehyde effective to produce an amine to aldehyde ratio of approximately 1 (e.g., 0.85 to 1.15). Without wishing to be bound by theory, it is believed that an amine to aldehyde ratio of approximately 1 would exhibit robust mechanical properties.

In some embodiments, the crosslinking reaction, for example, the crosslinking functional group(s), may be configured or selected to reach crosslinking completion in less than about 60 minutes, for example, less than about 30 minutes, less than about 15 minutes, less than about 10 minutes, or less than about 5 minutes, less than about 2 minutes, less than about 60 seconds, less than about 30 seconds, less than about 10 seconds, less than about 3 seconds, or less than about 1 second. In certain exemplary embodiments, inducing self-assembly of a peptide having a cysteine moiety with a polyethylene glycol hydrophilic polymer and glutaraldehyde chemical crosslinker molecule reduced crosslinking completion time from about 4 minutes to less than 2 minutes, for example, less than 90 seconds, or about 60 seconds.

Crosslinking completion may be a substantial completion, for example, at least about 70% crosslinking completion, at least about 75% crosslinking completion, at least about 80% crosslinking completion, at least about 85% crosslinking completion, at least about 90% crosslinking completion, at least about 95% crosslinking completion, at least about 98% crosslinking completion, or at least about 99% crosslinking completion.

Crosslinking completion and/or gelation time is generally related to the reaction kinetics and gel mechanics associated with the crosslinking density within the gel network. In general, gelation time is identified when loss modulus (G″) is higher than storage modulus (G′). Reaction kinetics and gel mechanical properties, such as gel stiffness, can be controlled by designing a formulation that provides selected rheological properties, such as loss modulus (G″) and storage modulus (G′). In exemplary embodiments, a concentration of the crosslinking agent may be selected to control reaction kinetics. A concentration of the hydrophobic polymer may be selected to control storage modulus (G′).

In some embodiments, the film may be formed of an interpenetrating network hydrogel. The network may be formed by combining the peptide, polymer, and crosslinker to produce a film having covalent crosslinking and non-covalent crosslinking. In some embodiments, the film may be formed of an interpenetrating network hydrogel comprising a first network and a second network. The first network may comprise covalent crosslinks and the second network may comprise a non-covalent crosslink, e.g., ionic or physical, crosslinks. In certain embodiments, the first network and the second network may be coupled, for example, covalently coupled.

The nature of the bonds between first and second networks may be determined using Fourier Transform Infrared (FTTIR) spectra or thermogravimetric analysis (TGA). The interpenetrating network film may exhibit enhanced mechanical properties, such as improved self-assembly and/or healing ability, therapeutic ability, increased fracture toughness, increased ultimate tensile strength, and/or increased rupture stretch.

The interpenetrating network hydrogel may be produced by combining, e.g., mixing, a covalently crosslinked first network and a non-covalently crosslinked network, e.g., ionically or physically crosslinked second network. The covalently crosslinked first network and non-covalently crosslinked second network may be combined, e.g., mixed at the molecular level. This combination may produce enhanced mechanical properties of the hydrogels. For example, while not wishing to be bound by theory, it is believed that fracture toughness may be enhanced by the following mechanism: the covalently crosslinked network may bridge the crack and stabilize deformation in the background, the chemical interactions between two networks may transfer the load over a large zone, and the ionic bonds between the ionically crosslinked network may break and provide inelastic deformation over the large zone around the root of the notch. Similarly, physical bonds between the physically crosslinked network may break and provide inelastic defamation over the large zone around the root of the notch.

In some embodiments, the film may further comprise a buffer comprising an effective amount of an ionic salt to induce the purified amphiphilic peptide to form a predetermined secondary structure. In the film, the purified amphiphilic peptide and the buffer may be in the form of the predetermined secondary structure. The predetermined secondary structure may comprise a structure preselected from at least one of a β-strand, β-sheet, an α-helix, and a random coil. For instance, the preselected structure may be or comprise a β-hairpin.

The film may comprise a hydrogel in the form of at least one of a cryogel, a dehydrated hydrogel, and a hydrated hydrogel.

The film may be porous. In some embodiments, the film may have at least 75% pores by volume, for example, 75% to 99% pores by volume, 75% to 85% pores by volume, 85% to 95% pores by volume, or 95% to 99% pores by volume, for example, about 75% pores, 80% pores, 85% pores, 90% pores, 91% pores, 92% pores, 93% pores, 94% pores, 95% pores, 96% pores, 97% pores, 98% pores, 99% pores, or more. The pores may be primarily microporous, for example, having an average pore size of less than about 50 nm, for example, having an average pore size of less than about 10 nm, or less than about 2 nm. In some embodiments, micropores may have an average pore size from about 0.1 nm to about 50 nm, for example, from about 0.1 nm to about 2 nm. In some embodiments, at least 50% of the pores may be microporous, for example, at least 60%, at least 70%, at least 80%, or at least 90% of the pores may be microporous. A remainder of the pores may be macroporous.

In other embodiments, the pores may be primarily nanoporous, for example, having an average pore size of less than about 500 nm, for example, having an average pore size from about 1 nm to about 500 nm or about 1 nm to about 100 nm. In some embodiments, at least 50% of the pores may be nanoporous, for example, at least 60%, at least 70%, at least 80%, or at least 90% of the pores may be nanoporous. A remainder of the pores may be microporous, microporous, or both.

In some embodiments, the pores may be primarily macroporous, for example, having an average pore size of greater than about 50 nm, for example, from about 50 nm to about 1 mm, from about 50 nm to about 10,000 nm, or from about 50 nm to about 1,000 nm. In some embodiments, at least 50% of the pores may be macroporous, for example, at least 60%, at least 70%, at least 80%, or at least 90% of the pores may be macroporous. A remainder of the pores may be microporous.

In some embodiments, at least some of the macropores may be open interconnected macropores. In some embodiments, at least some of the pores may be interconnected. At least 50%, 60%, 70%, 80%, 90%, 95%, or 99% of the pores may be interconnected. While not wishing to be bound by theory, it has been found that interconnectivity of at least some of the pores may improve function of the film composition. It is believed that without interconnectivity, water may become trapped within the gel. Interconnectivity of the pores is believed to permit or enable passage of water (and other compositions such as cells and compounds) in and out of the film structure.

The film may be at least partially cryogelated, lyophilized, or combinations thereof. The manner and/or amount of cryogelation and/or lyophilization may be effective to control porosity.

In some embodiments, the film may have a loss modulus (G′) of from about 0.1 Pa to about 10,000 Pa, for example, from about 1 Pa to about 5,000 Pa, from about 100 Pa to about 1,000 Pa, from about 1,000 Pa to about 5,000 Pa, or from about 5,000 Pa to about 10,000 Pa.

The film may have a storage modulus (G′) of from about 0.1 Pa to about 10,000 Pa, for example from about 1 Pa to about 5,000 Pa, from about 100 Pa to about 1,000 Pa, from about 1,000 Pa to about 5,000 Pa, or from about 5,000 Pa to about 10,000 Pa.

The film may have a thickness of from about 0.1 mm to about 100 mm when hydrated. For instance, the film may have a thickness of from about 0.1 mm to about 1.5 mm, about 0.5 mm to about 1.0 mm, about 1.0 mm to about 10 mm, about 10 mm to about 50 mm, or about 50 mm to about 100 mm when hydrated.

The film may have a thickness of from about 5 μm to about 100 μm when dehydrated. For instance, the film may have a thickness of from 5 μm to about 90 μm, about 30 μm to about 45 μm, about 45 μm to about 100 μm, about 100 μm to about 500 μm, or about 500 μm to about 1000 μm when dehydrated.

The film may be formulated to provide gel water absorbability. Water absorbability may be beneficial to remove excess exudates on a wound site. The film may have a gel mass swelling ratio (determined by dividing the mass of hydrated gel to the mass of dehydrated gel) of at least 2-fold, for example, at least 4-fold, at least 6-fold, at least 8-fold, at least 10-fold, at least 12-fold, at least 14-fold, at least 16-fold, at least 18-fold, or at least 20-fold.

The film may be capable of at least 5% strain, for example, at least 10% strain, at least 15% strain, at least 20% strain, or at least 25% strain. In particular, the film may be formulated to substantially maintain gel mechanics or mechanical properties under the target strain.

The film may be formulated to have a selected gel fraction. Gel fraction may be determined by dividing a second dehydrated weight (for example, after hydrating and dehydrating) by a first dehydrated weight (for example, a weight of the gel before hydrating). Gel fraction may provide an indication of peptide content of the gel and percent of the gel lost to diffusion. In some embodiments, the film may have a gel content of at least 50%, for example, at least 60%, at least 70%, at least 75%, at least 80%, at least 85%, at least 90%, at least 95%, or at least 99%.

The film may be formulated for topical, enteral, or parenteral administration. Enteral administration may comprise oral, sublingual, sublabial, buccal, or rectal application, as described in more detail below.

The film may be formed of an antimicrobial peptide. For instance, the film may be formulated for treatment of a microbial infection or elimination or inhibition of proliferation of a target microorganism, for example, a pathogenic microorganism. The film may be formulated for management or inhibition of a microbial bioburden. The film may be formulated for treatment of a fungal infection. The film may be formulated for treatment of a viral infection. The film may be formulated for treatment of a bacterial infection.

The film may be formulated for treatment of infected wounds and/or treatment or inhibition of biofilm. The film may be formulated for wound and/or biofilm management. The film may be formulated for tissue hydration, moisture management, and/or exudate management of wounds or tissues. The film may be formulated as a barrier, barrier dressing, and/or hemostat.

The film may be formulated to enable cell attachment. The film may be formulated to enable tissue adhesion. The film may be formulated to enable tissue regeneration. The film may be formulated to enable cell and tissue infiltration. Cell and tissue infiltration may comprise, for example, cell attachment, cell proliferation, and/or cell growth. The cell and or tissue infiltration may occur within at least 3 days of administration of the film, for example, within at least 2 days, within at least 1 day, or within at least 12 hours of administration of the film.

In some embodiments, at least 5% by volume of the film may be formulated to allow infiltration by cells, for example, at least 10% by volume, at least 25% by volume, or at least 50% by volume of the film may be formulation to allow infiltration of cells.

In some embodiments, the film may further comprise an active agent. The active agent may comprise, for example, at least one of an antibacterial composition, an antifungal composition, an antiviral composition, a hemostat, a growth factor, a cytokine, a chemokine, an anti-inflammatory composition, an analgesic composition, a local anesthetic composition, or a pain-relief composition.

The film may be thermally stable between −20° C. and 150° C., as described in more detail below. For example, the film may be sterilized by terminal and/or autoclave sterilization. The film may have a shelf-life of at least about 1-5 years at room temperature.

The film may be formulated to be physically stable, chemically stable, biologically stable, and/or non-biodegradable. In some embodiments, the film may be formulated to be biodegradable. The film may be formulated to substantially biodegrade within a predetermined period of time, for example, at least about 1 year, at least about 6 months, at least about 2 months, at least about 1 month, at least about 2 weeks, at least about 1 week, or at least about 1 day. The film may be formulated to be biodegradable by hydrolysis. The film may be formulated to be biodegradable by proteolysis.

In some embodiments, the film is in a hydrated state. As disclosed herein, the “hydrated state” may be at least 85% water by volume. The hydrated state may be at least 85% water by volume, for example, between 85-99% water by volume, or at least 85%, 92%, 95%, 97%, 99%, or more water by volume.

In some embodiments, the film is in a cryogel state. The cryogel may allow for cell and tissue integration. For example, without wishing to be bound by theory, it has been found that the film formulation in a cryogel state demonstrates superior cell and tissue integration within the film, improved biodegradation, and biocompatibility of the film when implanted. In some embodiments, the cryogel state may be at least 90% water by volume, for example, at least 92%, 95%, 97%, 99%, or more water by volume. In the cryogel state, the water may be contained in the pores of the film. When dehydrated, the cryogel may allow higher flexibility and lower fragility.

In some embodiments, the film is in a dehydrated state. The dehydrated state may be at least 50% less, 60% less, or 70% less water by volume than the hydrated or cryogel state. In some embodiments, the dehydrated state may be less than 25% water by volume, for example, less than 20%, 15%, 10%, 5%, or less water by volume.

In some embodiments, the film is capable of being dehydrated and rehydrated. For example, the film is capable of reversible dehydration or rehydration. The film may be capable of hydration or rehydration by interaction with a physiological fluid. The physiological fluid may be, for example, water, saline, and/or a bodily fluid.

The film may be capable of being hydrated or rehydrated in situ. For example, the film may be capable of being hydrated or rehydrated upon application at a target site of the subject. The film may come into contact with the physiological fluid upon application at the target site. In some embodiments, the film may be hydrated or rehydrated at a point of use, for example, prior to application.

The film may be substantially cytocompatible. The film may be substantially biocompatible. The film may be substantially free of a preservative.

### Kits Comprising the Film

Kits for producing the film are provided. The kit may comprise the polymer. The kit may comprise the self-assembling peptide. The kit may comprise a crosslinking agent. The kit may comprise an active agent. The kit may comprise a buffer comprising an effective amount of an ionic salt to induce the peptide to form the predetermined secondary structure.

The kit may comprise instructions to combine any one or more of the components, for example, at a point of use. For instance, the kit may comprise instructions to form the film by crosslinking the peptide with the polymer. The kit may comprise instructions to induce the peptide to form the predetermined secondary structure by combining the peptide with the buffer.

### Methods of Producing the Film

Methods of producing a film are disclosed herein. The methods may comprise crosslinking the self-assembling peptide and the polymer to form the film. The methods may comprise covalently, ionically, and/or physically crosslinking the self-assembling peptide with the polymer. The peptide and the polymer may be crosslinked by combining the peptide and polymer with an effective amount of a crosslinking agent.

The reaction may be allowed to proceed for a predetermined period of time. For example, the reaction may be allowed to proceed for at least about 60 minutes, for example, at least about 30 minutes, at least about 15 minutes, at least about 10 minutes, or at least about 5 minutes, at least about 2 minutes, at least about 60 seconds, at least about 30 seconds, at least about 10 seconds, at least about 3 seconds, or at least about 1 second. The reaction may reach completion in less than about 60 minutes, for example, less than about 30 minutes, less than about 15 minutes, less than about 10 minutes, or less than about 5 minutes, less than about 2 minutes, less than about 60 seconds, less than about 30 seconds, less than about 10 seconds, less than about 3 seconds, or less than about 1 second.

In some embodiments, the method may comprise crosslinking the peptide and the polymer at a point of use, for example, in situ.

The method may comprise inducing the peptide to form a predetermined secondary structure by combining the peptide with a buffer comprising an effective amount of an ionic salt. The peptide may be induced to form the predetermined secondary structure at a point of use, for example, in situ.

In some embodiments, the film may be produced in accordance with a Layer-by-Layer (LbL) method. LbL is a thin film fabrication technique that generally involves depositing alternating layers of oppositely charged materials. The films disclosed herein may be produced by depositing alternating layers, for example, the method may include depositing a self-assembling peptide layer and a biocompatible polymer layer. The layers may be repeated. In some embodiments, the methods may include depositing a crosslinking agent or crosslinker molecule layer. Alternatively, the crosslinking agent or crosslinker molecule may be incorporated in one or both of the self-assembling peptide layer and the biocompatible polymer layer. Each layer may be independently deposited in any of a variety of material depositing mechanisms, such as, dip coating, spin-coating, spray-coating, and others. The method may include depositing one or more functional layer, such as, a polyion layer, a metal layer, a ceramic layer, a nanoparticle layer, a biological molecule or active agent layer, a therapeutic agent layer, or others. In certain embodiments, the method may be used to produce an interpenetrating network hydrogel, for example, by depositing a layer corresponding to the first network having covalent crosslinking and a second layer corresponding to a second network having non-covalent crosslinking.

In some embodiments, the film may be produced by electrospinning. Electrospinning generally involves the use of electric force to draw charged threads of a polymer solution. Electrospinning may be used herein to draw charged threads of the biocompatible polymer. The method may comprise combining the biocompatible polymer thread with the self-assembling peptide and optional crosslinking agent or crosslinker molecule. Electrospinning may produce films with improved properties, such as improved porosity, cell attachment, cell proliferation, biodegradation, flexibility and plasticity, and/or drug encapsulation.

### Kits Comprising the Peptide Preparation

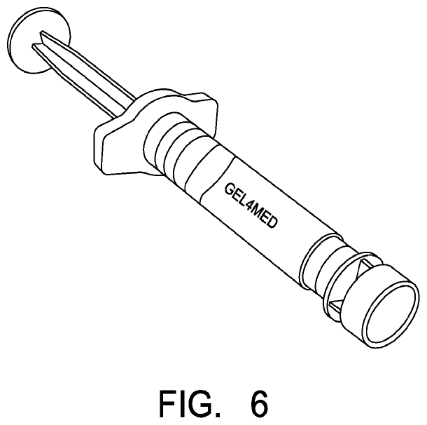

Kits comprising the peptide preparation are described herein. The kit may comprise the peptide preparation and a buffer solution. The buffer may be configured to induce self-assembly of the peptide prior to or concurrently with administration of the peptide. Each of the peptide preparation and the buffer may be included in a vial or chamber. For example, the kit may comprise a pre-filled packaging containing one or more of the preparation and the buffer. The kit may comprise one or more devices for use and/or delivery of the peptide preparation. The kit may comprise a mixing device. The kit may comprise a delivery device. In certain embodiments, the delivery device and/or mixing device may be the pre-filled packaging, for example, the kit may comprise a pre-filled syringe, spray bottle, ampule, or tube. FIG. 6 is a photograph of the preparation packaged in an end-use container. The exemplary end-use container of FIG. 6 is a pre-filled syringe. The end-use container may be employed as a delivery device or a mixing device. The kit and/or any component of the kit may be sterile or sterilized. For example, the kit and/or any component may be sterilized using autoclave sterilization, optionally terminal autoclave sterilization.

Any one or more component of the kit may be autoclavable. The packaged kit may be autoclavable. Any one or more component of the kit may be sterilized or sterile. For example, any one or more component of the kit may be sterilized by autoclave. The sterilized one or more component may be packaged in a substantially air-tight container. In some embodiments, the packaged kit may be sterilized, e.g., by autoclave.

In certain embodiments, the kit may comprise the purified peptide in a dried or powder form. For example, the purified peptide may be lyophilized. The kit may comprise a biocompatible solution to be combined with the purified peptide to produce the peptide preparation. In other embodiments, the kit may comprise instructions to combine the purified peptide with a biocompatible solution to produce the preparation. The kit may additionally comprise the buffer solution.

The kit may comprise instructions for use. In particular, the kit may comprise instructions to combine the buffer with the preparation, optionally in the mixing device, to form the hydrogel. A user may be instructed to combine the preparation and the buffer at the point of use. In some embodiments, the user may be instructed to combine the preparation and the buffer prior to administration or concurrently with administration. The user may be instructed to apply the preparation and the buffer to the target site separately.

The kit may additionally comprise instructions to store the kit under recommended storing conditions. For instance, the kit may comprise instructions to store the preparation or any component at room temperature (approximately 20-25° C.). The kit may comprise instructions to store the preparation or any component under refrigeration temperature (approximately 1-4° C.). The kit may comprise instructions to store the preparation or any component under freezer temperature (approximately 0 to −20° C.). The kit may comprise instructions to store the preparation or any component at body temperature (approximately 36-38° C.). The kit may comprise instructions to store the preparation or any component under cool and dry conditions.

The kit may additionally comprise an indication of expiration for the preparation or any component. The indication of expiration may be about 1 year after packaging. The indication of expiration may be between about 6 months and about 10 years after packaging, for example, between about 1 year and about 5 years after packaging.

The kit may comprise additional components for administration in combination with the preparation. In some embodiments, the kit may comprise instructions to combine the additional component prior to administration or concurrently with administration. The kit may comprise instructions to administer the preparation and the additional component to the target site separately. The additional component may be or comprise an antibacterial formulation, an antiviral formulation, an antifungal formulation, an anti-tumor formulation, an anti-inflammatory formulation, a cell culture media, a cell culture serum, an anti-odor formulation, an analgesic, a hemostat formulation, local anesthetic, or a pain-relief formulation. In particular embodiments, the kit may comprise a culture of cells for administration in combination with the preparation, as described herein. In some embodiments, the kit may further comprise a dressing, e.g., a topical dressing, a barrier dressing, and/or a wound dressing.

The kit may comprise one or more component configured to induce shear-thinning of the hydrogel. Mixing devices or delivery devices (described below) may be employed to induce shear-thinning of the hydrogel by mechanical agitation. The kit may comprise one or more component selected from a temperature control device, a pH control additive, an ion chelator composition, a solvent, a sound control device, a lyophilization device, and an air drying device to induce shear-thinning. For example, the kit may comprise a heater or cooler, a source of an acid or a base, a source of an ion chelator, a source of a solvent, a speaker or sound transmitter, a lyophilizer, or a compressed air dryer, or a fan.

### Mixing Devices

Mixing devices for preparation of a hydrogel at a point of care are disclosed herein. The device may be a multi-chamber device. In exemplary embodiments, the device may be a two-chamber device. The devices may include a first chamber for a peptide preparation. The preparation may comprise a self-assembling peptide in a biocompatible solution. The devices may include a second chamber for a buffer solution. The first chamber and the second chamber may be separated by a barrier provided to prevent fluid communication between the first chamber and the second chamber. The devices may, optionally, further comprise a mixing chamber. The mixing chamber may be fluidly connectable to the first chamber and the second chamber. Prior to mixing, the mixing chamber may be separated from the first chamber and/or the second chamber by a barrier. In other embodiments, the mixing device may be configured for direct mixing of the contents of the first and second chambers. In some embodiments, the devices may comprise a third chamber for an additional formulation or compound to be administered to the subject. The third chamber may be separated from the first chamber, the second chamber, and/or the mixing chamber. The third chamber may be fluidly connectable to the first chamber and/or the second chamber directly or via the mixing chamber.

The device may be a single use device. The device may be a multiple use device.

In an exemplary embodiment, each of the first, second, or third chamber may be a syringe barrel. Each barrel may have an associated plunger for agitation. Each barrel may be hermetically fitted to a coupling adapter, which forms the mixing chamber. The hermetic fitting may be, for example, a luer lock or luer taper connection.

The preparation and buffer may be agitated or otherwise mixed to form a homogenous or substantially homogenous mixture, inducing hydrogelation. In some embodiments, the preparation and buffer may be agitated by transferring the mixture back and forth between the first chamber and the second chamber. In some embodiments, the hydrogel exhibits shear-thinning properties, such that during agitation the mixture is substantially liquid. Upon settling, the mixture may form a solid or gel material.

In exemplary embodiments, the device may be configured to prepare a cell graft at a point of use. In use, the first chamber may comprise the cell preparation and the second chamber may comprise the peptide preparation. The cell preparation may comprise buffer. Alternatively, a third chamber may comprise buffer. Upon actuation the cell preparation and the peptide preparation may mix or contact, i.e. in the mixing chamber. The cells may be suspended in the peptide solution, forming a cell suspension comprising self-assembling peptides.

The cell preparation and the peptide preparation may be mixed with buffer, forming a buffer suspension. The buffer suspension may be agitated as described above, inducing self-assembly of the hydrogel. The buffer suspension may be agitated to disperse the cells, forming a homogenous or substantially homogenous mixture. The homogenous or substantially homogenous suspension may self-assemble to form a hydrogel cell graft.

The mixing device may be a static mixing device. A static mixer may generally comprise a device for substantially continuous mixing of the preparation without moving components. For example, the static mixer may comprise a cylindrical or rectangular housing with one or more inlet for each component to be mixed and a single outlet for the mixture. The static mixer may comprise a plate-type mixer.

The mixing device may generally be formed or lined with an inert, thermally-stable material. In certain embodiments, the material may be opaque and/or shatter resistant.

### Delivery Devices

In some embodiments, the kits may include a delivery device. For instance, the kits may include a syringe or catheter. The kits may include a dropper. The kits may include a spray, e.g. in conjunction with a bottle. The spray device may be, for example, a nasal spray. The kits may include a tube or ampule. The kits may include a film, for example. The type of delivery device may be selected based on a target indication. Additionally, the properties of the delivery device may be selected based on a target indication. For instance, a syringe for topical delivery of the preparation may have a larger passage than a syringe for injection of the preparation.

In some embodiments, the syringe may be used for topical application of the preparation. In other embodiments, the syringe may comprise a needle for parenteral application. The needle of the syringe may have a Birmingham system gauge between 7 and 34. The catheter may be used for parenteral application. The needle of the catheter may have a Birmingham system gauge between 14 and 26. The peptide may be formulated for administration through a particular gauge needle. For instance, the peptide may be selected to have a variable viscosity that will allow application of the preparation through a particular gauge needle.

In some embodiments, the spray bottle may be used for topical application of the preparation. The spray bottle may comprise a container for the preparation and a spray nozzle. The spray nozzle may be configured for targeted delivery to a target tissue. For instance, a spray nozzle for targeted delivery to an epithelial tissue may have a substantially flat surface and a spray nozzle for targeted delivery to an intranasal tissue may have a substantially conical surface. The spray nozzle may be configured to deliver a predetermined amount of the preparation. In some embodiments, the spray nozzle may be configured to deliver the preparation in substantially unidirectional flow, optionally, regardless of orientation of the spray bottle.

The spray nozzle may be configured to reduce retrograde flow. In certain embodiments, the spray nozzle may be spring-loaded. In other embodiments, the spray nozzle may be pressure actuated. The actuation pressure may be selected based on the variable viscosity of the preparation. For instance, the actuation pressure may be selected to be sufficient to dispense the hydrogel through the spray nozzle.

The film may be used for topical application of the preparation. The film may be saturated with the preparation. The film may be used as a barrier dressing and/or hemostat. In some embodiments, the film may accompany a barrier dressing.

The delivery device may be a single use device. The delivery device may be a multiple use device. The delivery device may include a first chamber for a peptide preparation. The preparation may comprise a self-assembling peptide in a biocompatible solution. The delivery device may include a second chamber for a buffer solution. The first chamber and the second chamber may be separated by a barrier provided to prevent fluid communication between the first chamber and the second chamber. The delivery device may be constructed and arranged for administration of the peptide preparation and the buffer solution simultaneously or substantially simultaneously. In some embodiments, the delivery device may comprise a third chamber for an additional formulation or compound to be administered to the subject. The third chamber may be separated from the first chamber and/or the second chamber.

The delivery device may generally be formed or lined with an inert, thermally-stable material. In certain embodiments, the material may be opaque and/or shatter resistant.

### Coated Medical or Surgical Devices

In some embodiments, medical or surgical tools may have at least a portion of an exterior surface coated with the preparations or hydrogels disclosed herein. The coating may enable the exterior surface of the tool to exhibit antimicrobial properties, reducing incidence of infection. The coating may enable the exterior surface of the tool to be biocompatible or cytocompatible, reducing rejection and adverse reaction from contact.

The surgical tool may be a surgical instrument. For example, the tool may be a grasper, such as forceps, clamp or occluder, needle driver or needle holder, a suture or suture needle, retractor, distractor, positioner, stereotactic device, mechanical cutter, such as scalpel, lancet, drill bit, rasp, trocar, ligasure, harmonic scalpel, surgical scissors, or rongeur, dilator, specula, suction tip or tube, sealing device, such as surgical stapler, irrigation or injection needle, tip and tube, powered device, such as drill, cranial drill, or dermatome, scopes or probe, including fiber optic endoscope and tactile probe, carrier or applier for optical, electronic, and mechanical devices, ultrasound tissue disruptor, cryotome, cutting laser guide, or a measurement device, such as ruler or caliper. Other surgical tools or instruments are within the scope of the disclosure.

The medical or surgical tool may be an implantable tool. For example, the medical or surgical tool may be an implantable device, such as, implantable cardioverter defibrillator (ICD), pacemaker, intra-uterine device (IUD), stent, e.g., coronary stent, ear tube, or eye lens. Other implantable tools are within the scope of the disclosure. The implantable medical or surgical tool may be a prosthetic or a portion of a prosthetic device, for example, a prosthetic hip, knee, shoulder, or bone or a portion of a prosthetic limb. The implantable medical or surgical tool may be a mechanical tool, such as a screw, rod, pin, plate, disk, or other mechanical support. The implantable medical or surgical tool may be a cosmetic tool, such as breast implant, calf implant, buttock implant, chin implant, cheekbone implant, or other plastic or reconstructive surgery implant. Other medical or implantable tools are within the scope of the disclosure.

The formulation and/or thickness of the coating may be selected to be temporary, for example, degrading within a pre-determined period of time, for example, less than about 3 months, less than about 1 month, or less than about 2 weeks. The formulation and/or thickness of the coating may be selected to be semi-permanent, for example, degrading within a predetermined period of time of between about 3 months and 3 years, or between about 6 months and 2 years. The formulation and/or thickness of the coating may be selected to be permanent, for example, having a lifespan of more than 2 years or more than 3 years, or having a lifespan longer than the predetermined period of time that the medical or surgical tool is in contact with the subject.

### Methods of Treatment by Administration of Peptide Hydrogels

In some embodiments, the preparations disclosed herein may be administered according to a predetermined regimen. The preparations disclosed herein may be administered daily, weekly, monthly, yearly, or bi-yearly.

The preparations disclosed herein may provide immediate release effects. For example, the onset of action of the active ingredient may be less than one minute, several minutes, or less than one hour.

The preparations disclosed herein may provide delayed release effects. For example, the onset of action of the active ingredient may be more than one hour, several hours, more than one day, more than several days, or more than one week.

The preparations disclosed herein may provide extended release effects. For example, the preparations may be effective for more than one day, more than several days, more than one week, more than one month, several months, or up to about 6 months.

The preparations disclosed herein may be administered in conjunction with a medical approach that treats the relevant disease or disorder or a symptom of the relevant disease or disorder. For example, the preparations may be administered in conjunction with a medical approach that is approved to treat the relative disease or disorder or a symptom of the relevant disease or disorder. The preparations may be administered in conjunction with a medical approach that is commonly used to treat the relevant disease or disorder or a symptom of the relevant disease or disorder.

The preparations disclosed herein may be administered in combination with a surgical treatment. The preparations disclosed herein may be administered to treat wounds associated with the surgical treatment and/or to prevent or treat biofilm.

The preparations disclosed herein may be administered in combination with an anti-inflammatory agent or treatment. Anti-inflammatory agent may refer to a composition or treatment which reduces or inhibits local or systemic inflammation. The anti-inflammatory agent may comprise, e.g., non-steroidal anti-inflammatory drugs (NSAID), antileukotrienes, immune selective anti-inflammatory derivatives (ImSAID), and/or hypothermia treatment.

The preparations disclosed herein may be administered in combination with an antibacterial, antiviral, and/or antifungal agent. Such agents may refer to compositions or treatments which eliminate or inhibit proliferation of bacterial, viral, and/or fungal organisms, respectively. Exemplary antibacterial agents include antibiotics and topical antiseptics and disinfectants. The antiviral agent may be a target-specific antiviral agent or a broad-spectrum antiviral agent (e.g., remdesivir, ritonavir/lopinavir). Exemplary local antiviral agents include topical antiseptics and disinfectants. Exemplary antifungal agents include antifungal antibiotics, synthetic agents (e.g., flucytosine, azoles, allylamines, and echinocandins), and topical antiseptics and disinfectants.

The preparations disclosed herein may be administered to treat a wound, for example, an acute, a sub-acute, or a chronic wound. The wound may be a surgical wound, laceration, burn wound, bite/sting wound, vascular wound (venous, arterial or mixed), diabetic wound, neuropathic wound, pressure wound, ischemic wound, moisture-associated dermatitis, or result from a pathological process. In certain embodiments, the preparations may be administered in an amount effective to treat diabetic foot ulcers (DFU). In certain embodiments, the preparations may be administered in an amount effective to treat gastrointestinal wounds, such as anal fistulas, diverticulitis, and ulcers. In particular, the preparations may be administered in an amount effective to promote infection free wound closures.

The preparations disclosed herein may be administered in combination with a treatment or agent to provide anesthesia and/or pain-relief, e.g., local anesthetic. “Anesthetic” may refer to a composition which provides temporary loss of sensation or awareness. The anesthetic may be a general anesthetic (e.g., GABA receptor agonists, NMDA receptor antagonists, or two-pore potassium channel activators) or a local anesthetic (e.g., ester group agents, amide group agents, and naturally derived agents).

The preparations may be administered in combination with an analgesic or pain-relief agent. “Analgesic” may refer to a composition for systemic treatment or inhibition of pain. The analgesic may comprise an anti-inflammatory agent or an opioid.

The preparations disclosed herein may be administered in combination with a hemostat agent. “Hemostat” may refer to a tool or composition to control bleeding. Exemplary hemostat compositions include collagen-based agents, cellulose-based agents, and chitosan-based agents.

The preparations disclosed herein may be administered in combination with a treatment or agent to enhance cell or tissue graft therapy. In certain embodiments, the preparations disclosed herein may be administered in combination with a treatment or agent to prevent or inhibit cell death and/or control or reduce inflammation. The preparations disclosed herein may be administered in combination with cell culture media or cell culture serum.

The administered peptide hydrogels may have an immediate local effect. For instance, the administered preparations may provide localized wound healing or injury treatment effects by closing the wound or filling a void. In certain embodiments, the administered hydrogels may have a systemic effect. For instance, the administered hydrogels may enable cell migration or communication between cell grafts and environmental cells, resulting in a systemic effect. In other embodiments, the administered hydrogels may enable delivery of cell products or byproducts, resulting in a systemic effect. The administered peptide hydrogels may have antimicrobial, antiviral, and/or antifungal properties at a localized site of administration. In other embodiments, the administered peptide hydrogels may provide systemic antimicrobial, antiviral, and/or antifungal properties.

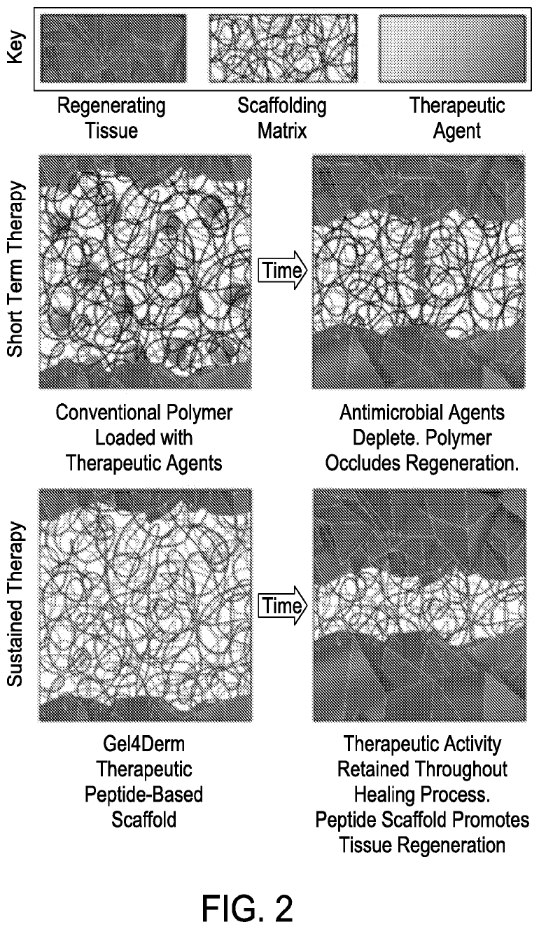

The administered peptide hydrogels may have long-term, sustained antimicrobial, antiviral, and/or antifungal properties at a localized site of administration. The peptide may be designed to form a hydrogel having a direct contact antimicrobial, antiviral, antifungal effect. Thus, the hydrogel may eradicate microorganisms which directly contact the hydrogel at the target site. The hydrogel may be substantially free of encapsulated antimicrobial, antiviral, and/or antifungal agents. Furthermore, the local antimicrobial, antiviral, and/or antifungal effect may persist as long as the hydrogel is in contact with the target tissue. FIG. 2 includes images which show sustained antimicrobial, antiviral, and/or antifungal effect at the target site.

To provide a systemic antimicrobial, antiviral, and/or antifungal effect, the peptide hydrogel may additionally comprise encapsulated antimicrobial, antiviral, and/or antifungal agents. Administration of such a hydrogel may generally provide: (1) local antimicrobial, antiviral, and/or antifungal treatment by direct contact as previously described, and (2) systemic antimicrobial, antiviral, and/or antifungal treatment as a vehicle of an encapsulated therapeutic agent.

The preparations disclosed herein may be formulated as a hemostat, debridement agent, or barrier dressing (e.g., antimicrobial, antifungal, or antiviral barrier dressing). The preparations may be formulated for wound treatment. Exemplary wounds which may be treated by administration of the preparation include partial and full thickness wounds (e.g., pressure sores, leg ulcers, diabetic ulcers), first and second degree burns, tunneled/undermined wounds, surgical wounds (e.g., associated with donor sites/grafts, tissue and cell grafts, Post-Moh's surgery, post laser surgery, podiatric, sound dehiscence), trauma wounds (e.g., abrasions, lacerations, burns, skin tears), gastrointestinal wounds (e.g., anal fistulas, diverticulitis, ulcers), and draining wounds. The preparations may be formulated for administration to a predetermined target tissue, for example, mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue, or a biological fluid.

### Methods of Treatment of Microbial Infection

The preparation may be formulated to provide antimicrobial properties upon administration at a target site. For example, the self-assembled polymeric hydrogel may have antimicrobial properties. As disclosed herein, “antimicrobial” properties may refer to an effect against a microbial population, e.g., killing or inhibiting one or more microorganism from a microbial population. Thus, methods of treating a microbial infection or killing or inhibiting proliferation of a target microorganism are disclosed herein. “Proliferation” may generally refer to the metabolic or reproductive activity of an organism. Methods of reducing or eliminating a microbial contamination are disclosed herein. Methods of management of a microbial bioburden are disclosed herein. The methods may generally comprise administering the preparation in an amount effective to promote deactivation of a target microorganism. In particular, a preparation comprising about 3.0% w/v or less of the peptide, for example, 155% w/v or less, or 1.0% w/v or less, may provide antimicrobial properties at a target site.

The methods may comprise identifying a subject as being in need of treatment for a microbial contamination, colonization, or infection. In general, a microbial colonization or infection may be induced by proliferation of a pathogenic microorganism (disease-causing microorganism). The microbial contamination may be identified by presence of one or more microorganism. In some embodiments, the methods may be employed for prevention or treatment of a microbial colonization or infection. The microbial colonization may refer to an established colony of one or more microorganism. The microbial infection may refer to an established colony of one or more microorganism which has been diagnosed by a clinical assessment. The microbial colonization or infection may be localized or systemic. In general, a microbial contamination may develop into a microbial colonization or infection if adequate treatment is not provided.

The preparation may be administered in an amount effective to treat biofilm or a microbial infection. The methods may generally comprise administering the preparation in an amount effective to promote deactivation of a pathogenic microorganism. In certain embodiments, the pathogenic microorganism may be a pathogenic bacterial organism. For example, the preparations and methods may be effective at promoting deactivation of broad-spectrum (gram-positive and gram-negative) bacteria. The pathogenic microorganism may be a species of a genus selected from Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

The preparation may be administered in combination with a surgical procedure. The methods may comprise administering the preparation in an amount effective to sterilize at least 90% of the target microorganism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the target microorganism at the target site. Exemplary target sites include epithelial tissue, gastrointestinal system tissue, respiratory system tissue, cardiac system tissue, nervous system tissue, reproductive system tissue, ocular tissue, auditory tissue, and bloodstream. Epithelial tissue may include, for example, epidermis, dermis, hair, and nail. However, additional target sites may be treated by the methods disclosed herein. As disclosed herein, sterilize may refer to any process that eliminates, removes, kills, or deactivates the microorganism at the target site.

### Methods of Treatment of Fungal Infection

The preparation may be formulated to provide antifungal properties upon administration at a target site. For example, the self-assembled polymeric hydrogel may have antifungal properties. As disclosed herein, “antifungal” properties may refer to an effect against a fungal population, e.g., killing or inhibiting one or more organism from a fungal population. Thus, methods of treating a fungal infection or inhibiting proliferation of a fungal organism are disclosed herein. The methods may generally comprise administering the preparation in an amount effective to promote deactivation of a fungal organism. Methods of reducing or eliminating a fungal contamination are disclosed herein. In exemplary embodiments, a preparation comprising about 3.0% w/v or less of the peptide, for example, 1.5% w/v or less, or 1.0% w/v or less, may provide antifungal properties at a target site.

The methods may comprise identifying a subject as being in need of treatment for a fungal contamination, colonization, or infection. In certain embodiments, the preparation may be administered in an amount effective to treat at least one of biofilm, Tinea corporis, Candidiasis, Blastomycosis, Coccidioidomycosis, Histoplasmosis, Cryptococcosis, Paracoccidioidomycosis, Aspergillosis, Aspergilloma, Meningitis, Mucormycosis, Pneumocystis pneumonia (PCP), Talaromycosis, Sporotrichosis, and Eumycetoma of the subject. In some embodiment, the fungal organism may be a species of a genus selected from Aspergillus and Candida.

The preparations and methods may be effective at promoting deactivation of broad-spectrum (sporulating and non-sporulating) fungal organisms. The preparation may be administered in an amount effective to treat a fungal infection associated with or inhibit proliferation of at least one of Aspergillus clavatus, Aspergillus fischerianus, Aspergillus flavus, Aspergillus fumigatus, Aspergillus niger, Trichophyton mentagrophytes, Trichophyton rubrum, Microsporum canis, Candida albicans, Candida auris, Candida parapsilosis, Candida tropicalis, Blastomyces dermatitidis, Coccidioides immitis, Coccidioides posadasii, Cryptococcus gatti, Cryptococcus neoformans, Histoplasma capsulatum, Paracoccidioides brasiliensis, Pneumocystis jirovecii, Mucormycetes rhizopus, Mucormycetes mucor, Mucormycetes lichtheimia, Talaromyces marneffei, Sporothrix schenckii, Acremonium strictum, Noetestudina rosatii, Phaeoacremonium krajdenii, Pseudallescheria boydii, Curvularia lunata, Cladophilaophora bantiana, Exophiala jeanselmei, Leptosphaeria senegalensis, Leptosphaeria tompkinsii, Madurella grisea, Madurella mycetomatis, Pyrenochaeta romeroi, Trichosporon asahii, Cladosporium herbarum, and Fusarium sporotrichioides.

The preparation may be administered in combination with a surgical procedure. The methods may comprise administering the preparation in an amount effective to sterilize at least 90% of the fungal organism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the fungal organism at the target site. Exemplary target sites may include epithelial tissue, oral tissue, esophageal tissue, tracheal tissue, pulmonary tissue, cardiac tissue, kidney tissue, ocular tissue, and bloodstream. Epithelial tissue may include, for example, epidermis, dermis, hair, and nail. However, additional target sites may be treated by the methods disclosed herein. As disclosed herein, sterilize may refer to any process that eliminates, removes, kills, or deactivates the fungal organism at the target site.

### Methods of Treatment of Biofilm

The preparation may be formulated for treatment of biofilm. Thus, the methods disclosed herein may comprise treatment of biofilm. Treatment of biofilm may generally comprise eliminating at least a portion of biofilm or inhibiting biofilm formation. Administration of the preparation may have an effect against a biofilm population, for example, killing or inhibiting one or more organism in a biofilm community. In general, the charged peptide polymer hydrogel may deconstruct the polymicrobial fungal and bacterial biofilm barrier upon contact. While not wishing to be bound by theory, it is believed the preparations disclosed herein may be selected to dismantle extracellular matrix of the biofilm population, exposing fungal, viral, and microbial organisms of the biofilm to the cationic peptide of the hydrogel. The peptide hydrogel may be effective by destroying microbes, fungi, and viral organisms within biofilms. The preparation may be administered as an antifungal, antimicrobial, and/or antiviral peptide to destroy fungi, microorganisms, and/or viral organisms, e.g., in a biofilm population, through cell lysis.

Methods of management of biofilm are also disclosed herein. For example, the methods may be employed for prevention of biofilm. The preparation may be administered to a target tissue having a population of biofilm or identified as prone to developing biofilm, e.g., a wound or wounded tissue. The preparation may be administered in response to tissue contamination or odor.

The methods may generally comprise administering the preparation in an amount effective to promote treatment of biofilm and/or deactivation of a biofilm population. The biofilm population may comprise bacterial organisms, for example, gram-positive and/or gram-negative bacterial organisms. The biofilm population may comprise fungal organisms, for example, sporulating and/or non-sporulating fungal organisms. Thus, the preparation may provide treatment of biofilm by the antimicrobial and/or antifungal properties and methods described above. In certain embodiments, the biofilm population may comprise viral organisms. The preparation may provide treatment of biofilm by antiviral properties and methods described herein.

The preparation may be formulated as a biofilm removal agent. In some embodiments, the preparation may be administered to a target tissue for removal of biofilm. For example, the preparation may be administered for debridement of the biofilm and/or biofilm-infected tissue.

### Methods of Treatment of Viral Infection

The preparation may be formulated to provide antiviral properties upon administration at a target site. For example, the self-assembled polymeric hydrogel may have antiviral properties. As disclosed herein, “antiviral” properties may refer to an effect against a viral population, e.g., killing or inhibiting one or more organism from a viral population. Thus, methods of treating a viral infection or inhibiting proliferation of a viral organism are disclosed herein. The methods may generally comprise administering the preparation in an amount effective to promote deactivation of a viral organism. Methods of reducing or eliminating a viral contamination are disclosed herein. In exemplary embodiments, a preparation comprising about 3.0% w/v or less of the peptide, for example, 1.5% w/v or less, or 1.0% w/v or less, may provide antiviral properties at a target site.

The methods may comprise identifying a subject as being in need of treatment for a viral contamination, colonization, or infection. In certain embodiments, the preparation may be administered in an amount effective to treat at least one of a respiratory viral colonization or infection (e.g., associated with rhinovirus, influenza, coronavirus, or respiratory syncytial virus), a viral skin infection (e.g., associated with molluscum contagiosum, herpes simplex virus, or varicella-zoster virus), a foodborne viral infection (e.g., associated with hepatitis A, norovirus, or rotavirus), a sexually transmitted viral infection (e.g., associated with human papilloma virus, hepatitis B, genital herpes, or human immunodeficiency virus), and other viral infections (e.g., associated with Epstein-Barr virus, West Nile virus, or viral meningitis) of the subject.

The preparation may be administered in combination with a surgical procedure. The methods may comprise administering the preparation in an amount effective to sterilize at least 90% of the viral organism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the viral organism at the target site or systemically. In some embodiments, the methods may comprise administering the preparation in an amount effective to sterilize 100% of the viral organism at the target site or systemically. In certain embodiments, the preparation may be administered in an amount effective to treat biofilm or kill or deactivate a biofilm population containing a viral organism.

Exemplary target sites may include epithelial tissue, oral tissue, esophageal tissue, tracheal tissue, pulmonary tissue, cardiac tissue, kidney tissue, ocular tissue, and bloodstream. However, additional target sites may be treated by the methods disclosed herein. As disclosed herein, sterilize may refer to any process that eliminates, removes, kills, or deactivates the viral organism at the target site.

### Methods of Administration of Peptide Hydrogels

The peptide hydrogels may be administered by any mode of administration known to one of skill in the art. The method of administration may comprise selecting a target site of a subject and administering the preparation to the target site. In certain embodiments, the methods may comprise mixing the peptide with a buffer configured to induce self-assembly of the peptide to form the hydrogel. In general, the peptide may be mixed with the buffer prior to administration. However, in some embodiments, the peptide may be combined with the buffer at the target site.

The target site may be any bodily tissue or bloodstream. In some embodiments, the target site may be epithelial tissue, gastrointestinal system tissue, respiratory system tissue, cardiac system tissue, nervous system tissue, reproductive system tissue, ocular tissue, or auditory tissue. The route of administration may be selected based on the target tissue. Exemplary routes of administration are discussed in more detail below.

In some embodiments, the methods may comprise identifying a subject in need of administration of the preparation. The methods may comprise imaging a target site or monitoring at least one parameter of the subject, systemically or at the target site. Exemplary parameters which may be monitored include temperature, pH, response to optical stimulation, and response to dielectric stimulation. Thus, in some embodiments, the method may comprise providing optical stimulation or dielectric stimulation to the subject, optionally at the target site, for measuring a response. The response may be recorded, optionally in a memory storing device. In general, any parameter which may inform a user of a need or desire for administration of the preparation may be monitored and/or recorded. The methods may comprise imaging the target site or monitoring at least one parameter of the subject prior to administration of the preparation, concurrently with administration of the preparation, or subsequent to administration of the preparation. For example, the methods may comprise imaging the target site or monitoring at least one parameter of the subject after an initial dose and before a potential subsequent dose of the preparation.

In certain embodiments, the preparation may be administered responsive to the measured parameter being outside tolerance of a target value. The preparation may be administered automatically or manually in response to the measured parameter.

The preparation may be formulated for topical, parenteral, or enteral administration. The preparation may be formulated for systemic administration. Various pharmaceutically acceptable carriers and their formulations are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42.2 S, 1988; Aulton, M. and Taylor, K., Aulton's Pharmaceutics: The Design and Manufacture of Medicines, 5th Edition, 2017; Antoine, A., Gupta M. R., and Stagner, W. C., Integrated Pharmaceutics: Applied Preformulation, Product Design, and Regulatory Science, 2013; Dodou K. Exploring the Unconventional Routes—Rectal and Vaginal Dosage Formulations, The Pharmaceutical Journal, 29 Aug. 2012.

### Parenteral Administration of Peptide Hydrogels

In certain embodiments, the hydrogels may be administered parenterally. In general, parenteral administration may include any route of administration that is not enteral. The preparation may be administered parenterally via a minimally invasive procedure. In particular embodiments, the parenteral administration may include delivery by syringe, e.g., by needle, or catheter. For instance, the parenteral administration may include delivery by injection. The parenteral administration may be intramuscular, subcutaneous, intravenous, or intradermal. The shear-thinning ability of the hydrogels may allow distribution through small lumens, while still providing minimally invasive treatment.

The method may comprise applying mechanical force to the hydrogel for parenteral administration. The hydrogel may be thinned by applied mechanical force, for example, pressure applied by a syringe to administer the preparation by injection. In particular, the pressure applied to administer the preparation through a needle or catheter may be sufficient to shear thin the hydrogel for application.

The peptide hydrogels may be administered parenterally to any internal target site in need thereof. For instance, cardiac tissue, nervous tissue, connective tissue, epithelial tissue, or muscular tissue, and others, may be the target site. The peptide hydrogels may be administered parenterally to a target site of a solid tumor. In exemplary embodiments, antifungal treatment of pulmonary tissue may be provided by parenteral administration of the peptide hydrogels described herein.

### Topical Administration of Peptide Hydrogels

In certain embodiments, the hydrogels may be administered topically. In general, topical administration may include any external or transdermal administration. For instance, the target site for administration may be an epithelial tissue. In particular embodiments, the topical administration may be accompanied by a wound dressing or hemostat.

The preparation may be administered topically with a delivery device. For instance, the preparation may be administered topically by spray, aerosol, dropper, tube, ampule, film, or syringe. In particular embodiments, the preparation may be administered topically by spray. The spray may be, for example, a nasal spray. Spray parameters which may be selected for administration include droplet size, spray pattern, capacity, spray impact, spray angle, and spray diameter. Thus, the methods may comprise selecting a spray parameter to correlate with the target site or target indication. For instance, a smaller spray diameter may be selected for administration to a small wound. A specific spray angle may be selected for administration to a target site which is difficult to reach. A denser spray pattern or larger droplet size may be selected for administration to a moist target site.

Exemplary droplet sizes may be between 65 μm to 650 μm. For instance, fine droplets having an average diameter of 65 μm to 225 μm, medium droplets having an average diameter of 225 μm to 400 μm, or coarse droplets having an average diameter of 400 μm to 650 μm may be selected. The spray pattern may range from densely packed droplets to sparse droplets. The spray diameter may range from less than 1 cm to 100 cm. For instance, spray diameter may be selected to be between less than 1 cm and 10 cm, between 10 cm and 50 cm, or between 50 cm and 100 cm. Spray angle may range from 0° to 90°. For instance, spray angle may be selected to be between 0° and 10°, between 10° and 45°, or between 45° and 90°.

In some embodiments, the preparation may be administered topically with a film. The film may be a rigid, semi-flexible, or flexible film. In certain embodiments, the flexible or semi-flexible film may be configured to adopt a topological conformation of the target site. In general, the film may be in the form of a substrate saturated with the preparation or hydrogel. The substrate may be rigid, semi-flexible, or flexible. The film may be administered as a barrier dressing and/or hemostat. The preparation may be administered topically with a film and accompanied by a barrier dressing.

The peptide formulated as a saturated film or barrier dressing may provide antimicrobial, antiviral, and/or antifungal treatment by direct contact with target population, as previously described. Conventional antimicrobial wound dressings rely on traditional antibiotics and function merely as a vehicle for antibiotic agents. However, the peptide hydrogel saturated film or barrier dressing described herein may be designed to provide a biophysical mode of cell-membrane disruption against broad-spectrum (gram-positive and gram-negative) bacterial cultures. Thus, the antimicrobial, antiviral, and/or antifungal peptide hydrogel saturated film or barrier dressing may generally avoid concerns around minimum inhibitory bacterial concentrations typical to conventional small molecule loaded polymers. Instead, the peptide hydrogel disclosed herein may be designed to exert toxicity against gram-positive and gram-negative bacteria (including antibiotic resistant strains) while remaining cell-friendly, non-inflammatory, and non-toxic by selecting amino acid charge ratio of the peptide. Similarly, the peptide hydrogel disclosed herein may be designed to exert toxicity against fungal organisms (e.g., sporulating and non-sporulating fungal organisms) and/or viral organisms. The saturated film or barrier dressing disclosed herein may provide a temporary extracellular matrix scaffold for tissue regeneration.

The peptide hydrogels may be administered topically to any target site in need thereof. Wound healing, e.g., diabetic wound healing, is described herein as one exemplary embodiment. However, it should be understood that many other topical target sites and treatments are envisioned, for example, as previously described above. The wounds may include acute, sub-acute, and chronic wounds. The wound may be a surgical wound or ischemic wound. Chronic wounds such as venous and arterial ulcer wounds or pressure ulcer wounds, and acute wounds, such as those caused by trauma may be treated. In some embodiments, the preparation may be formulated as a film, barrier dressing, and/or hemostat. Administration of the preparation may accompany a barrier dressing and/or hemostat.

Treatment and/or management or inhibition of biofilm is described herein as another exemplary embodiment. Tissue hydration is described herein as another exemplary embodiment. Moisture management and/or exudate management of wounds or tissues is described herein as another exemplary embodiment. Tissue debridement is described herein as another exemplary embodiment. The preparation may be administered topically as a prophylactic, for example, in association with a wound. The preparation may be administered topically as an analgesic, for example, to a chronic wound or site of biofilm.

### Enteral Administration of Peptide Hydrogels

In certain embodiments, the hydrogels may be administered enterally. In general, enteral administration may include any oral or gastrointestinal administration. For instance, the target site for administration may be an oral tissue or a gastrointestinal tissue. In particular embodiments, the enteral administration may be accompanied by food or drink. The preparation may be administered on a substantially empty stomach. In some embodiments, water is administered to the subject after administration of the preparation. In some embodiments, several hours are waited prior to food consumption after administration.

Such enteral preparations may be formulated for oral, sublingual, sublabial, buccal, or rectal application. Oral application formulations are generally prepared specifically for ingestion through the mouth. Sublingual and sublabial formulations, e.g., tablets, strips, drops, sprays, aerosols, mists, lozenges, and effervescent tablets, may be administered orally for diffusion through the connective tissues under the tongue or lip. Specifically, formulations for sublingual administration may be placed under the tongue and formulations for sublabial administration may be placed between the lip and gingiva (gum). Sublabial administration may be beneficial when the dosage form comprises materials that may be corrosive to the sensitive tissues under the tongue. Buccal formulations may generally be topically held or applied in the buccal area to diffuse through oral mucosa tissues that line the cheek. Rectal application may be achieved by inserting the formulation in the rectal cavity, either with or without the assistance of a device. Device-assisted application may include, for example, delivery via an applicator or an insertable applicator, catheter, feeding tube, or delivery in conjunction with an endoscope or ultrasound. Suitable applicators include liquid formulation bulbs and launchers and solid formulation insertable applicators.

For any of the routes of administration disclosed herein, the methods may comprise administering a single dosage of the preparation. The site of administration may be monitored for a period of time to determine whether a booster or subsequent dosage of the preparation is to be administered. For example, the methods may comprise monitoring the site of administration. A parameter of the subject, optionally at the target site, may be monitored as previously described. The subject may be monitored hourly, every 2-3 hours, every 6-8 hours, every 10-12 hours, every 12-18 hours, or once daily. The subject may be monitored daily, every other day, once every few days, or weekly. The subject may be monitored monthly or bi-monthly. In certain embodiments, the subject may be monitored for a period of up to about 6 months. For example, the subject may be monitored for about 1 month, about 2 months, about 3 months, about 4 months, about 5 months, or about 6 months.

The methods may comprise administering at least one booster or subsequent dosage of the preparation. For example, the methods may comprise administering a booster dosage to the target site at least about 1 day, 2 days, 3 days, 4 days, 5 days, 6 days, 7 days, 8 days, 9 days, 10 days, 11 days, 12 days, 13 days, or 14 days after the first dosage. The methods may comprise administering a booster dosage 1 week, 2 weeks, 3 weeks, 4 weeks, 5 weeks, or 6 weeks after the first dosage. The methods may comprise administering a booster dosage at least 6 months or 1 year after the first dosage. In certain embodiments, at least a portion of the hydrogel may be present at the target site at the time of the booster dosage. In other embodiments, the hydrogel may be fully metabolized or otherwise eliminated from the target site at the time of the booster dosage.

Methods of Biological Material Delivery with Peptide Hydrogels

Methods of administering biological material to a subject are disclosed herein. The methods may generally include suspending the biological material in a hydrogel. The biological material may be combined with a preparation comprising a self-assembling peptide in a biocompatible solution and a buffer comprising an effective amount of an ionic salt and a biological buffering agent to induce self-assembly of the hydrogel. The methods may comprise administering an effective amount of the biological material, the preparation, and the buffer (optionally in hydrogel form) to a target site of the subject. Suspending the biological material with the preparation or the buffer will generally produce a liquid suspension. Combining the preparation with the buffer may trigger gelation of the suspension into a hydrogel comprising the biological material.

The biological material to be administered may include biological fluids, cells, and/or tissue material. In some embodiments, one or more biological material administered may be synthetic. For instance, the biological fluids may be or include synthetic fluids. In other embodiments, the biological material may be obtained from a donor. The biological material may be autologous (obtained from the recipient subject). The biological material may be allogeneic (obtained from a donor subject of the same species as the recipient subject) or xenogeneic (obtained from a donor subject of a different species as the recipient subject).

The self-assembled hydrogel may have a physical structure substantially similar to the native extracellular matrix of the biological material, allowing the gel to serve as a temporary scaffold to promote cell growth, function, and/or viability. In particular, the self-assembled hydrogel may have a similar properties, including, for example, pore size, density, hydration, charge, rigidity, etc., to the native extracellular matrix of the biological material. The properties may be selected responsive to the biological material type.

The self-assembled hydrogel may have a selected degradation rate. The degradation rate may be selected responsive to the target site of implantation or administration. The self-assembled hydrogel properties may be selected to promote migration of cells to the hydrogel environment. The self-assembled hydrogel properties may be selected to promote cell protection in a hostile environment. The self-assembled hydrogel properties may be selected to promote anchoring of the biological material within the hydrogel, for example, as with cells that will not engraft onto host tissue. The self-assembled hydrogel properties may be selected to promote migration of cell products or byproducts or tissue-derived material to the hydrogel environment, for example, growth factors, exosomes, cell lysates, cell fragments, or genetic material. In an exemplary embodiment, the self-assembled hydrogel properties may be selected to control differentiation of cells, e.g., progenitor cells or stem cells, e.g., mesenchymal stem cells.

The properties of the self-assembled hydrogel may be controlled by designing the peptide. For example, the peptide may include functional groups that provide one or more selected physical property. The properties may be controlled by selecting the composition of media or buffer. For example, the media may include serum or be substantially free of serum. For example, the buffer may have a net positive charge, be net neutral, or have a net negative charge. In some embodiments, the functional group may be configured to alter peptide net charge or counterions when the peptide is suspended in the solution.

The administration of cells and cell products, byproducts, tissue, or tissue-derived material at the target site may be controlled by altering the release properties of the hydrogel. In some embodiments, the release properties may be engineered by controlling one or more of the expression of extracellular matrix or protein motifs, the presence or absence of fusion proteins, the net charge of the peptides, the presence or absence of cationic particles or peptides, the presence or absence of anionic particles or peptides, buffers, salts, peptide concentration, peptide purity, and the presence or absence of peptide counterions. The properties may be engineered to deploy cells at the target site. The properties may be engineered to deploy cell products or byproducts at the target site, for example, delivery of exosomes, growth factors, genetic material, RNA, siRNA, shRNA, miRNA, etc.

The self-assembled hydrogel may be designed to have cell protective properties. In particular, the self-assembled hydrogel may be designed to be protective against foreign microorganisms, e.g., pathogenic microorganisms. The self-assembled hydrogel may be designed to be protective against immune attack from environmental immune cells, for example, by providing a physical barrier or biochemical modulation. The antimicrobial and/or protective properties of the hydrogel may not substantially affect the viability, growth, or function of the engrafted cells.

The protective properties of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions.

The suspension may be designed to have a substantially physiological pH level. The suspension may have a pH level of between about 4.0 and 9.0. In some embodiments, the suspension may have a pH level of between about 7.0 and 8.0. The suspension may have a pH level of between about 7.3 and 7.5. The substantially physiological pH may allow administration of the suspension at the time of preparation. In some embodiments, the suspension may be prepared at a point of care. The methods may comprise suspending the cells in the peptide solution, optionally, agitating the suspension to provide a substantially homogeneous distribution of the cells, and administering the suspension at a point of care. The administration may be topical or parenteral, as described herein.

Biofabrication of Biological Material Grafts with Peptide Hydrogels

Methods of preparing biological material grafts in vitro for administration in vivo are disclosed herein. The methods may include self-assembly of a liquid suspension comprising cells into a peptide scaffold matrix in vitro. The self-assembled higher order structure may be administered to a target site of the subject.

Methods of preparing biological material grafts in vivo are disclosed herein. The methods may comprise administering a liquid suspension comprising the biological material for self-assembly into a higher order structure at a target site.

The methods may include biofabrication of the biological material graft at a point of care, as described in more detail below.

The hydrogels disclosed herein have gelation kinetics which are fast enough to ensure biological material becomes substantially homogeneously incorporated within the matrix. In particular, the gelation kinetics are sufficiently fast to afford an even distribution of encapsulated cells to allow reproducible control over cell density within the matrix. Additionally, the hydrogels disclosed herein have a construct that can be introduced in vivo and remain localized at the point of administration, for example, even without a spatial cavity. The localization of the hydrogel upon administration can limit or inhibit leakage of the cell construct to neighboring tissue.

The methods may comprise suspending the biological material in the preparation, optionally, agitating the suspension to provide a substantially homogeneous or non-homogeneous distribution of the biological material, and administering the suspension at a point of care. In some embodiments, the suspension may be agitated to provide a substantially homogeneous distribution of the biological material. In other embodiments, the suspension may be prepared or agitated to provide a non-homogeneous suspension, for example, comprising clusters or spheroids of the biological material.

Prior to engrafting, the cells may be cultured in vitro. Cell culture protocols generally vary by cell type. The conditions of the cell culture protocol may be selected based on the cell type. In exemplary embodiments, the cells may be autologous, allogeneic cells, or xenogeneic cells. The cultured cells may be suspended in water and/or media. In some embodiments, the methods disclosed herein may comprise collecting or harvesting cells from an organism. For instance, the methods disclosed herein may comprise collecting or harvesting cells from the subject. The methods disclosed herein may comprise collecting or harvesting a tissue graft from an organism, e.g., the subject.

The suspension may include a peptide configured to self-assemble in response to an external stimulus. The suspension and/or peptide may be engineered to express a desired property. For example, the suspension and/or peptide may be designed to express shear-thinning and/or antimicrobial properties.

In some embodiments, a buffer solution may be added to the suspension or a portion of the suspension to induce hydrogelation prior to or concurrently with administration. The hydrogel may form a homogenous or substantially homogenous cell matrix. The cell matrix may be administered to a target site as a solid or gel, optionally with shear-thinning properties as previously described.

The cells may be cultured in the cell graft in vitro for a predetermined period of time prior to administering the cell suspension to the subject. The period of time may range from several minutes, to several hours, to several days. The culture period may be selected based on cell type and target application. In other embodiments, the cells may be administered immediately after suspending or engrafting. The cells may be cultured in situ in the implanted cell graft.

The suspension and/or peptide may be engineered to express a desired property. In certain embodiments, the suspension and/or peptide may be engineered to protect cells from hostile environments. In particular, the suspension and/or peptide may be engineered to protect the cells from environments with a high microbial burden, hostile immune cells, or environmental proteins. The suspension and/or peptide may be engineered to increase cell viability. The suspension and/or peptide may be engineered to control differentiation, control cell fate in situ, control cell fate in vivo, control cell fate ex vivo, or control cell fate in vitro. The suspension and/or peptide may be engineered to increase cell attachment to the matrix or increase cell attachment and/or migration in the environment. The suspension and/or peptide may be engineered to decrease apoptosis, for example, by providing cell attachment and/or biological modulation.

The suspension and/or peptide may be engineered to achieve the results described above by altering the expression of protein motifs or the net charge of the peptides. The hydrogel properties may be engineered by controlling one or more of the expression of extracellular matrix or protein motifs, the presence or absence of fusion proteins, the net charge of the peptides, the presence or absence of cationic particles or peptides, the presence or absence of anionic particles or peptides, buffers, salts, peptide concentration, peptide purity, the presence or absence of peptide counterions, the presence or absence of specialized proteins, and the presence or absence of specialized small or large molecules.

Mixing devices for biofabrication of cell grafts at a point of care are disclosed herein. The devices may include a first chamber for a cell preparation. The cell preparation may comprise cells suspended in water, media, or buffer. The devices may include a second chamber for a peptide preparation, and optionally a third chamber for a buffer, as previously described.

### EXAMPLES

The function and advantages of these and other embodiments can be better understood from the following examples. These examples are intended to be illustrative in nature and are not considered to be limiting the scope of the invention.

### Example 1: Fabrication of Films from Chemically Modified Self-Assembling Peptides

Chemical crosslinking of the peptide gels through covalent bonds using two methods of crosslinking: (i) cysteine-based crosslinking and (ii) Schiff based crosslinking, was explored. For cysteine-based crosslinking, the peptide sequence was modified to include cysteine at one or both ends of the peptide sequence. The sequences and cysteine modified sequences are shown in Table 6.

TABLE 6
Peptide Sequences

| Peptide Sequence | Abbreviation |
|---|---|
| VKVRVRVRV(dP)PTRVRVRVKV-NH₂ | G4M-6R |
| VKVKVKVKV(dP)PTKVKVKVKV-NH₂ | GAM-0R |
| CVKVRVRVRV(DP)PTRVRVRVKV-NH₂ | G4M-1 Cys |
| Ac-CVKVRVRVRV(dP)PTRVRVRVKVC-NH₂ | G4M-2 Cys |

Peptides with cysteine moieties at the N-terminus or both N and C-terminus were designed to form additional disulfide bonds within the gel network. Tris (2-carboxyethyl) phosphine (TCEP), which is a strong reducing agent, was added to prevent any preformed disulfide crosslinking prior to disulfide bond formation. Disulfide crosslinking was initiated later by adjusting pH and temperature of the formulation.

To prepare the samples, peptide stock solutions (500 μl) at 3 w/v %, with or without adding 10 μl of TCEP, were prepared in deionized water and added to a 24-well plate. Buffer solution at pH 7.4 (NaCl 100 mM, BTP 33.4 mM) was added to the peptide solution samples. The samples were then incubated at room temperature (RT) overnight.

To enhance peptide-gel mechanical properties, as well as the cross-linkages within the gel network, a Schiff-base reaction was introduced. Schiff-base reactions refer to the crosslinking reaction between the substances containing carbonyls (aldehydes and ketones) with other groups (primary amine, hydroxylamine, etc.). In this strategy, a multi-amine-functionalized polyethylene glycol (PEG 4-arm amine, PEG4NH₂) and glutaraldehyde were used to initiate the reaction.

First, the effect of varying polymer and crosslinker contents on gel mechanical properties was examined via dynamic mechanical analysis (DMA). More specifically, PEG-4-arm-amine (at 1.25 wt %, 2.5 wt %, and 5 wt %) and glutaraldehyde concentration (at 0.25 wt %, 0.5 wt %, and 1.0 wt %) were varied, as shown in Table 7.

TABLE 7
Formulation Compositions

| | | | | Concentration of |
|---|---|---|---|---|
| | MW | | | functionality |
| Component | (g/gmole) | Functionality | wt(%) | (mM) |
| PEG4NH₂ | 2,000 | 4 | 5.00 | 100 |
| | | | 2.50 | 50 |
| | | | 1.25 | 25 |
| Glutaraldehyde | 100.11 | 2 | 2.00 | 400 |
| | | | 1.00 | 200 |
| | | | 0.50 | 100 |
| Peptide | 2398 | 8 | 0.75 | 25 |

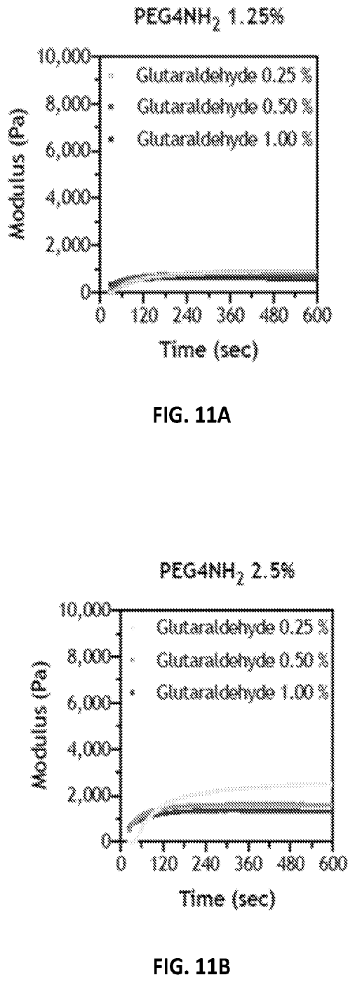

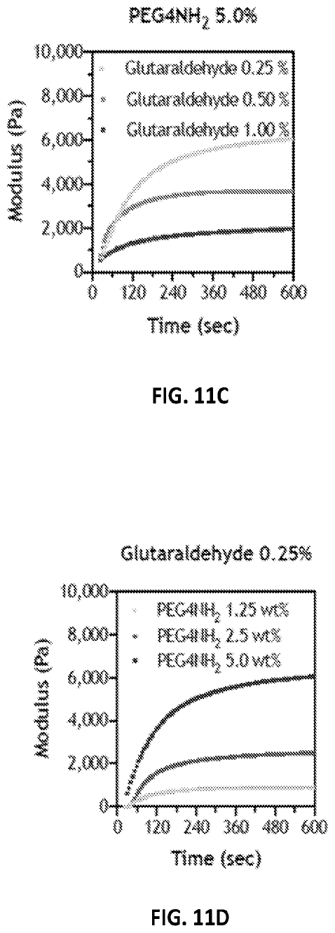

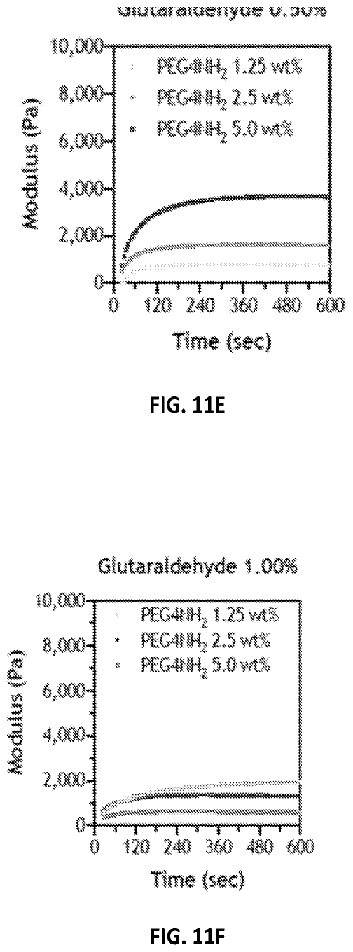

The gelation time and gel mechanics of varying formulation were investigated via rheology (TA Instruments, HR10) under time sweep mode (5% strain, 1 Hz for 10 minutes). PEG4NH₂ and glutaraldehyde may be controlled to determine reaction kinetics and resulting gel mechanics. In general, gelation time is identified when loss modulus (G″) is higher than storage modulus (G′). The rheological assessment showed that storage modulus of the hydrogel gradually increased over time in all conditions, which demonstrates crosslinking. The results are shown in the graphs of FIGS. 11A-11C.

As shown in FIGS. 11A-11C, when the polymer PEG4NH₂ contents were maintained constant and glutaraldehyde concentration was increased, the gelation time was shortened from about 240 seconds to about 60 seconds. Accordingly, crosslinking kinetic may be controlled by controlling concentration of the crosslinking agent (glutaraldehyde).

Additionally, when increasing PEG4NH₂ content, a higher gel storage modulus was observed under all conditions (FIGS. 11D-11F). It was surprisingly discovered that a higher glutaraldehyde content contrarily resulted in a lower gel stiffness. The phenomenon is due to the impaired ratio between amine and aldehyde concentration on each component. Accordingly, there is an optimal concentration of crosslinking agent (glutaraldehyde). While not wishing to be bound by theory, it is believed that an amine to aldehyde ratio of one will produce a hydrogel having the most robust mechanical properties.

In the field of polymer chemistry, gelation time is highly related to the reaction kinetics and gel mechanics is associated with crosslinking density within gel network. Collectively, these rheological data suggested that glutaraldehyde concentration directs reaction kinetics and PEG4NH₂ content affects the resulting gel stiffness.

Accordingly, crosslinking agent and hydrophobic polymer concentration can be controlled to control gel mechanical properties and obtain a desired gel handleability.

### Example 2: Structural and Functional Characterization of Films

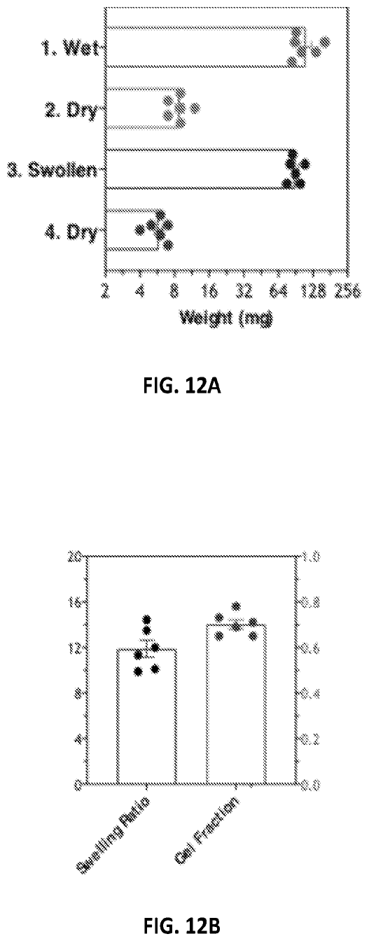

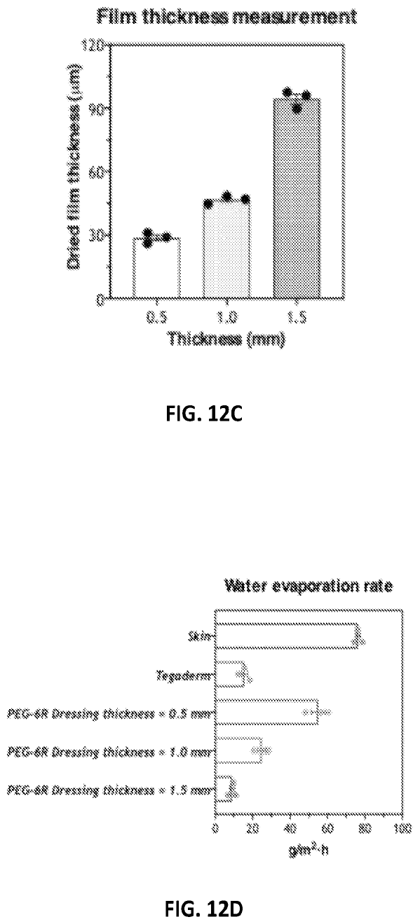

Structural and functional properties of the films were tested. The results are shown in the graphs of FIGS. 12A-12D.

To study gel water absorbability, which is an important dressing feature for absorbing wound exudates, gel swelling ratio was measured. Experimentally, gel mass swelling ratio is derived by dividing the mass of swollen (hydrated) gel to the mass of dried (dehydrated) gel. The gels of example 1 were determined to have a swelling ratio of about 12-fold (FIGS. 12A-12B). Accordingly, these gels are highly water-swollen, which can be beneficial to remove excess exudates on the wound site.

Gel fraction was also measured to evaluate reaction/gelation efficiency. Gel fraction is derived by dividing a second dried weight to a first dried weight. The gels of example 1 were determined to have a gel fraction about 75% (FIGS. 12A-12B). Accordingly, the potential peptide content within gel network in the reaction was 75% of the gel. The remaining (25%) is believed to have diffused during the swelling process.

Film thickness is an important parameter when producing a film which can be easily swollen, breathable, and easily applied on the wound site. Hydrated gels as described in example 1 (PEG4NH₂ 5%, glutaraldehyde 0.5%, and peptide 0.75%) were prepared at different thicknesses, from 0.5 to 1.5 mm. Hydrated gel thickness was measured. The gels were vacuum dried to measure dehydrated film thickness. As shown in FIG. 12C, the dehydrated gel film thicknesses were 30 μm, 45 μm, and 90 μm, corresponding to the hydrated gels at 0.5 mm, 1.0 mm, and 1.5 mm, respectively.

Water evaporation rate of the different gels was tested when applying film on skin and the results were compared to pure skin and Tegaderm (distributed by 3M) as controls. The results are shown in the graph of FIG. 12D.

The films were shown to have beneficial structural and functional properties.

### Example 3: Bioactivity and Functional Characterization of Rehydrated Films

To ensure that the crosslinking did not negatively affect the antimicrobial properties of the peptide sequence (G4M-6R), the antimicrobial effectiveness of the several films (before drying, after drying, and after rehydration) was investigated. Various final film formulations were tested. It was shown that the antimicrobial effectiveness correlates with the peptide content in the final formulation.

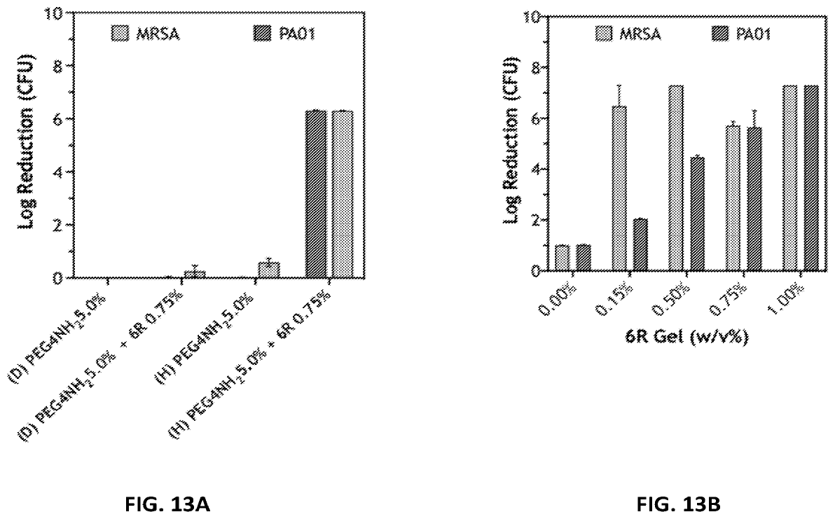

Methicillin-resistant Staphylococcus aureus (MRSA) and Pseudomonas aeruginosa (PA01) were cultured on agar plates at concentrations of at least 10⁶ CFU and antimicrobial properties of the hydrogel or the film were evaluated following modified ASTM E2315 method and Japanese antimicrobial testing method. The results are shown in the graphs of FIGS. 13A-13B.

The hydration state of the gel dressing was observed to be critical to the gel dressing antimicrobial efficacy, as shown in the graph of FIG. 13A. This gel formulation had high antimicrobial efficacy in the hydrated state, but not in the dehydrated state for all the conditions. This suggests that the gel dressing should be produced and maintained in the hydrated state to keep gel antimicrobial properties.

It is theorized that the low antimicrobial properties in the dehydrated state are due to collapsing of the self-assembled structures that are critical for the antimicrobial properties. It is believed that improvements can be made by optimizing the drying technique (e.g., controlled lyophilization process). This was briefly explored by developing lyophilized formulations.

The relationship between gel antimicrobial effect and peptide content of the hydrogel was evaluated. The results are shown in FIG. 13B, the antimicrobial effect was enhanced both on MRSA and PA01 with increasing peptide content within gel dressing. The result verified the hypothesis that the antimicrobial effectiveness of the final formulation is correlated with peptide concentration in the formulation.

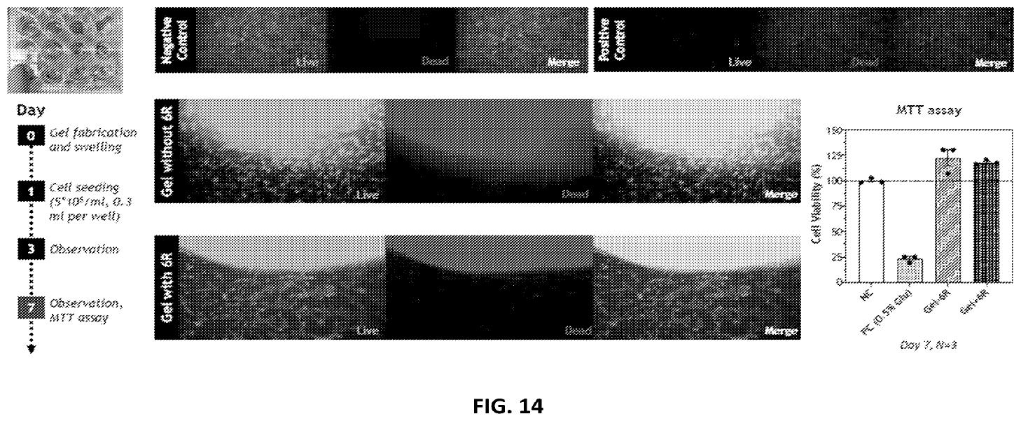

The gel formulation of example 1 (PEG4NH₂ 5%, glutaraldehyde 0.5%, and peptide 0.75%) was used for the following biocompatibility tests. To investigate in vitro cytotoxicity of G4M gel dressing, the direct contact method was used following ISO 10993 standard. Briefly, the gel dressing (100 μl) was prepared in the center of a 24 well-plate and swelled in PBS for a day. Then, 3T3 fibroblasts (500,000 cells/ml; 1 ml each well) were suspended in media (DMEM/high glucose with 10% FBS) and added to the gel dressing, as shown in FIG. 14.

The cells were incubated for 7 days, followed by live/dead staining and an MTT assay to determine the cytotoxicity of the gel dressing. Live/dead staining was used to evaluate the cytotoxicity effects qualitatively. On day 7, the gel dressings with and without peptide both exhibited good cell viability without any significant number of dead cells. Additionally, the results from the MTT assay matched the results from the live/dead staining. More precisely, cell viability was observed to be about 125% in the group without peptide and about 120% in the groups with the peptide (no significant difference).

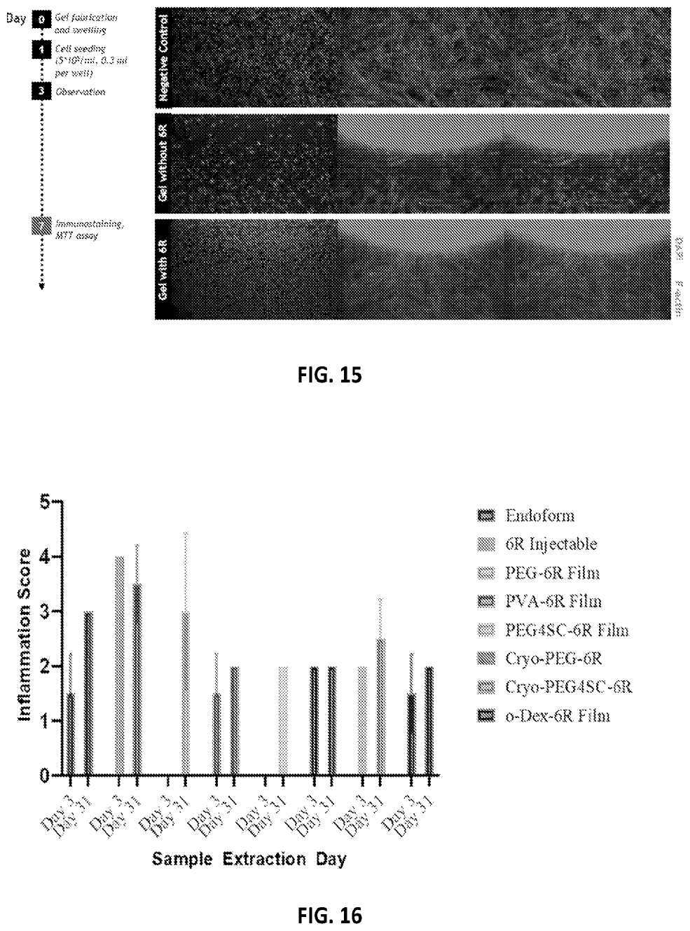

Additionally, cell morphological structure was examined via staining the F-actin and DAPI (FIG. 15). In this experiment, there were three groups including a negative control, a PEG gel with peptide, and a PEG gel without peptide. Results were collected on day 7. The fibroblasts cultured with gel exhibited a spreading morphology similar to the control group.

Accordingly, biocompatibility is not compromised by the film.

### Example 4: In Vivo Biocompatibility of Films

In vivo biocompatibility of films was evaluated using a subcutaneous dorsal mouse model. A summary of formulations used in this biocompatibility study is presented in Table 8. Cryogel (porous) forms of the bulk films were tested as well as degradation modifications of the bulk films to observe biocompatibility, tissue integration and degradation.

TABLE 8
Film Formulations

| Group | Dressing Code | Formulation |
|---|---|---|
| 1 | Endoform | Endoform dermal template collagen |
| | | dressing (control) |
| 2 | 6R (flowable) | 0.75% peptide |
| 3 | PEG-6R (film) | PEG4NH₂ 5%, glutaraldehyde 0.5%, |
| | | peptide 0.75% |
| 4 | PVA-6R | PVA 5%, peptide 0.75% |
| 5 | PEG4SC-6R | PEG4SC 1.25%, peptide 0.75% |
| | (film) | |
| 6 | Cryo-PEG-6R | PEG4NH₂ 5%, glutaraldehyde 0.5%, |
| | (film) | peptide 0.75% |
| 7 | Cryo-PEG4SC-6R | PEG4SC 1.25%, peptide 0.75% |
| | (film) | |
| 8 | o-Dex-6R (film) | o-Dex 5%, peptide 0.75% |

Biocompatibility and degradability of the films in vivo was determined by subcutaneous implantation into the dorsal surface of mice. Eight CD-1 mice each received a 1 cm² volume of the gel implant placed under the skin. The implantation site was marked and closed with sutures. The animals were monitored closely for 31 days. The animals were evaluated for any visible signs of toxicity or immune response.

Four animals from each group were humanely euthanized. The implanted samples were harvested on days 3 and 31 and processed for histopathological analysis using H&E staining. Different histological samples were evaluated by a board-certified pathologist for biocompatibility assessment.

On day 0, mice were anesthetized using isoflurane and brought to a surgical plane. The dorsal area of each mouse was shaved and thoroughly cleaned using surgical scrub. A midline incision was made in the shaved area of approximately 10-12 mm in length. Two pockets (one to the left and one to the right) were made in the subcutaneous area using blunt forceps.

The test disks (5 mm diameter) were placed on a small, moist cotton. Tipped applicator or forceps were used to carefully insert the implants to each pocket placing the implant horizontally flat in each pocket. Wounds were closed using skin staples. Staples were removed 1 week following surgery for the mice with endpoint of day 31. For the injectable hydrogel, a 20 μL volume was directly deposited subcutaneously using an 18G needle.

Inflammation score was measured. The results are shown in the graph of FIG. 16. As shown in FIG. 16, all groups had inflammatory cell infiltrates, with the greatest degree of severity in group 2 (peptide injectable). The PVA peptide, PEG4SC peptide, cryogel, and o-Dex-peptide groups had less inflammatory cell infiltrates comparable with Endoform (control).

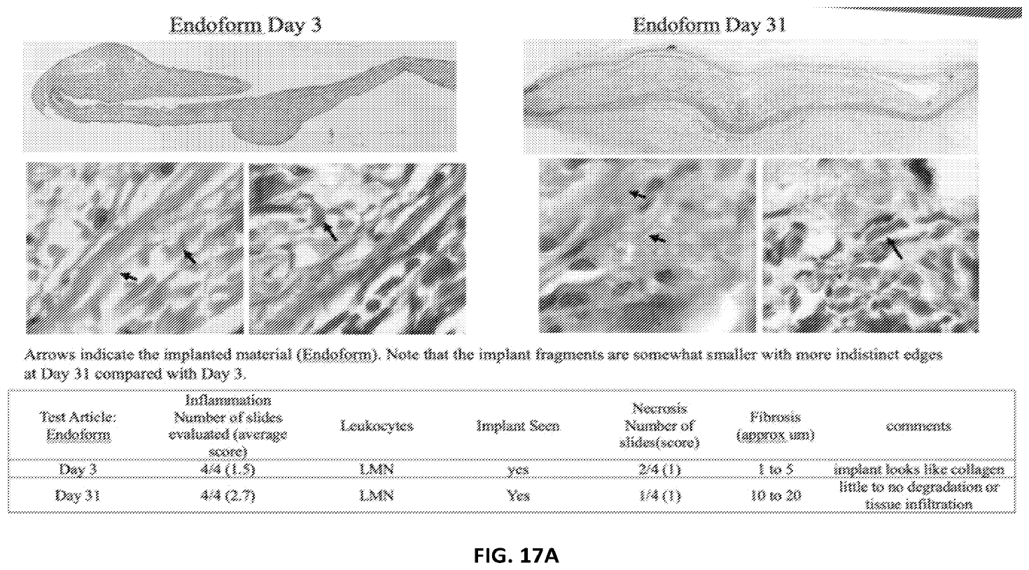

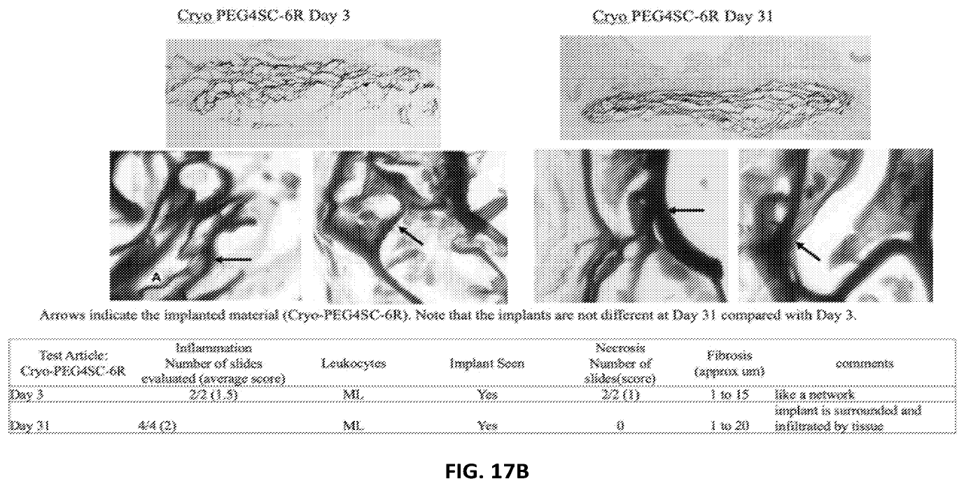

Inflammatory infiltrates for certain groups were similar intensity at days 3 and 31. Inflammatory infiltrates for other groups increased at day 31. Microporous films increased biocompatibility and tissue integration properties. Images are shown in FIGS. 17A-17B.

Accordingly, even though the samples had a positive inflammation score, the samples showed good biocompatibility in vivo.

### Example 5: Genipin Crosslinking

A genipin crosslinked film was prepared in accordance with the following protocol. A 6-arginine peptide was dissolved in culture grade DI water at 3 wt %. Buffer was added to a pH of 7.4 (NaCl 100 mM and BTP 33.4 mM). A genipin solution at 1 mM or 10 mM was made in PBS and ethanol (95:5 v/v, pH=7.4). The genipin solution was filtered through 0.22 μm filter. 100 μl of the peptide solution was added to a 1.5 ml tube and 100 μl of the genipin solution was added on top. The tubes were incubated at room temperature for 72 hours to complete the crosslinking reaction. The resulting composition is suitable for crosslinking with a biocompatible polymer to form a film.

### Example 6: Glutaraldehyde Crosslinking

Glutaraldehyde-mediated crosslinking was used to improve mechanical properties of the peptide gels. It is important to note that non-crosslinked glutaraldehyde can be toxic, and the crosslinked product should be rinsed multiple times before application. Parameters, such as pH, concentration and temperature, can greatly affect the crosslinking reaction and therefore, these parameters should be kept constant for any comparison.

Glutaraldehyde crosslinking was performed with different buffers including NaCl, NaNO₃ and CaCl₂ at 100 mM (all the buffers had 33.4 mM BTP) at pH 7.4. Peptide (6-arginine peptide and 0-arginine peptide) was dissolved in DI water at 1.5 wt % or 3 wt %. 300 μl of peptide solution was loaded into a 1 ml syringe immediately after mixing (syringe 1). 300 μl of buffer solution was loaded to another syringe (Syringe 2). Syringe 1 and 2 were connected using a luer-lock connector and mixed at least 20 times. Then, the solution was transferred to one syringe, capped and autoclaved at 121° C. for 30 minutes. The solution was then cooled to 60° C. and cast on Teflon mold. Glutaraldehyde in H₂O solution was diluted to 5% from 50% solution using DI and added to the cast peptide/buffer solution. The molds were covered and incubated at room temperature or higher temperature (i.e., 60° C.) overnight. The resulting composition is suitable for crosslinking with a biocompatible polymer to form a film.

### Example 7: Biodegradability and Gelation of Film Formulations

A biodegradable film was formed from succinimidyl carbonate (SC) modified 4-arm PEG (1.25%) and peptide hydrogel (0.75%).

A non-biodegradable film was formed from NH₂ modified 4-arm PEG (3.75%), glutaraldehyde (0.75%), and peptide hydrogel (0.75%).

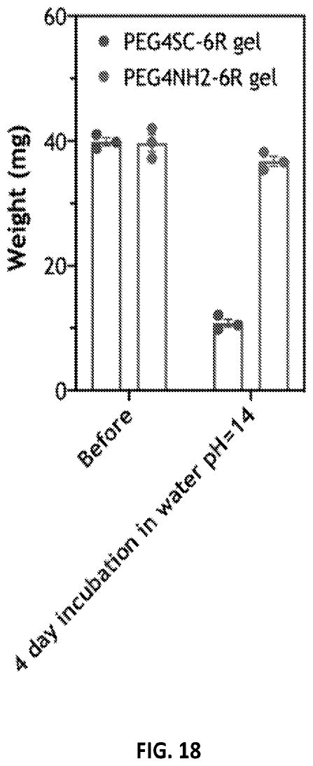

The biodegradability of the films after a 4-day incubation in water at a pH of 14 is shown in the graph of FIG. 18. As shown in FIG. 18, the SC modified film exhibited some biodegradation in the form of a reduction in weight (mg), while the NH₂ modified film did not substantially biodegrade.

The formulations were also tested for gelation. Both formulations formed gels. A comparative film was formed from NH₂ modified 2-arm PEG (7.5%), glutaraldehyde (0.5%), and peptide hydrogel (0.75%). The comparative formulation did not form a gel. It is theorized that the 4-arm PEG is more efficient at forming a gel than a 2-arm counterpart.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

Supplemental Table 1: Select Self-Assembling Peptide Sequences

| SEQ ID | | | |
|---|---|---|---|
| and | | | Modification |
| length | Description | Sequence | and location |
| SEQ ID | G4D_0R; | VKVKVKVKVPPTKVKVKVKV | D-amino |
| NO: 1 | Synthetic | | acid |
| (20 aa) | Peptide | | (10)...(10) |
| SEQ ID | G4D_6R_v1; | VKVRVRVRVPPTRVRVRVKV | D-amino |
| NO: 2 | Synthetic | | acid |
| (20 aa) | Peptide | | (10)...(10) |
| SEQ ID | G4D_6R_v1_ | VKVRVRVRVPPTRVRVRVKVGRGDSP | D-amino |
| NO: 3 | RGD; | | acid |
| (26 aa) | Synthetic | | (10)...(10) |
| | Peptide | | |
| SEQ ID | G4D_6R_v4; | VRVRVKVRVPPTRVEVRVRV | D-amino |
| NO: 4 | Synthetic | | acid |
| (20 aa) | Peptide | | (10)...(10) |
| SEQ ID | G4D_6R_v2; | VRVRVRVKVPPTKVRVRVRV | D-amino |
| NO: 5 | Synthetic | | acid |
| (20 aa) | Peptide | | (10)...(10) |
| SEQ ID | G4D_6R_v2_ | RGDSVRVRVRVKVPPTKVRVRVRV | D-amino |
| NO: 6 | RGD; | | acid |
| (24 aa) | Synthetic | | (14)...(14) |
| | Peptide | | |
| SEQ ID | G4D_6R_v1_ | VKVRVRVRVPTRVRVRVKVGKLTWQELYQLKYKGI | D-amino |
| NO: 7 | VG; | | acid |
| (36 aa) | Synthetic | | (10)...(10) |
| | Peptide | | |
| SEQ ID | G4D_6R_v1_ | VKVRVRVRVPPTRVRVRVKVRPKPQQFFGLM | D-amino |
| NO: 8 | SP; | | acid |
| (31 aa) | Synthetic | | (10)...(10) |
| | Peptide | | |
| SEQ ID | G4D_6R_v1_ | VKVRVRVRVRVRVRVKV | |
| NO: 9 | Ntrn; | | |
| (17 aa) | Synthetic | | |
| | Peptide | | |
| SEQ ID | G4D_2R_v1; | VKVKVRVKVPPTKVKVRVKV | D-amino |
| NO: 10 | Synthetic | | acid |
| (20 aa) | Peptide | | (10)...(10) |
| SEQ ID | G4D_6R_V3; | VKVRVRVRYNGTRVRVRVKV | |
| NO: 11 | Synthetic | | |
| (20 aa) | Peptide | | |
| SEQ ID | G4D_V1_ | LKKTETQGVKVRVRVRVPPTRVRVRVKV | D-amino |
| NO: 12 | LKKTETQ; | | acid |
| (28 aa) | Synthetic | | (18)...(18) |
| | Peptide | | |
| SEQ ID | G4D_V1_ | SDKPGVLVRVRVRVPPTRVRVRVKV | D-amino |
| NO: 13 | SDKP; | | acid |
| (25 aa) | Synthetic | | (15)...(15) |
| | Peptide | | |
| SEQ ID | G4D_6R_V2_ | RGDSVRVRVRVKVPPTKVRVRVRV | D-amino |
| NO: 14 | RGD; | | acid |
| (24 aa) | Synthetic | | (14)...(14) |
| | Peptide | | |
| SEQ ID | G4D_0R_E_ | VKVKVKVKVPPTKVEVKVKV | D-amino |
| NO: 15 | MAX8; | | acid |
| (20 aa) | Synthetic | | (10)...(10) |
| | Peptide | | |
| SEQ ID | G4D_HLT2; | VLTKVKTKVPPTKVEVKVLV | D-amino |
| NO: 16 | Synthetic | | acid |
| (20 aa) | Peptide | | (10)...(10) |
| SEQ ID | G4D_HLT2_ | VLTKVKTKVPPTKVEVKVLVGGGGRGDV | D-amino |
| NO: 17 | RGD; | | acid |
| (28 aa) | Synthetic | | (10)...(10) |
| | Peptide | | |
| SEQ ID | G4D_6R_v1_ | RGDSVKVRVRVRVPPTRVRVRVKV | D-amino |
| NO: 18 | RGD; | | acid |
| (24 aa) | Synthetic | | (14)...(14) |
| | Peptide | | |
| SEQ ID | G4D_6R_v1_ | IKVAVGVKVRVRVRVPPTRVRVRVKV | D-amino |
| NO: 19 | IKVAV; | | acid |
| (26 aa) | Synthetic | | (16)...(16) |
| | Peptide | | |
| SEQ ID | G4D_6R_v1_ | YIGSRGVKVRVRVRVPPTRVRVRVKN | D-amino |
| NO: 20 | YISGR; | | acid |
| (26 aa) | Synthetic | | (16)...(16) |
| | Peptide | | |
| SEQ ID | Synthetic | VLTKVKTVPPTKVEVKVL | D-amino |
| NO: 21 | Peptide 21 | | acid |
| (18 aa) | | | (9)...(9) |
| SEQ ID | Synthetic | VEVSVSVEVPPTEVSVEVEVGGGGRGDV | D-amino |
| NO: 22 | Peptide 22 | | acid |
| (28 aa) | | | (10)...(10) |
| SEQ ID | Synthetic | VEVSVSVEVPPTEVSVEVEV | D-amino |
| NO: 23 | Peptide 23 | | acid |
| (20 aa) | | | (10)...(10) |

1. A preparation comprising:
a biocompatible polymer having at least two functional groups capable of undergoing covalent crosslinking with a peptide group; and
a purified amphiphilic peptide comprising a folding group having a plurality of charged amino acid residues and hydrophobic amino acid residues arranged in a substantially alternating pattern and a turn sequence, and at least one functional group available for covalent crosslinking,
the purified amphiphilic peptide being capable of undergoing covalent crosslinking with the biocompatible polymer by a chemical crosslinker molecule or a coupling chemical agent to form a porous film.

2. A preparation comprising:
a biocompatible polymer capable of undergoing ionic or physical crosslinking with a peptide group; and
a purified amphiphilic peptide comprising a folding group having a plurality of charged amino acid residues and hydrophobic amino acid residues arranged in a substantially alternating pattern and a turn sequence, and at least one functional group available for crosslinking,
the purified amphiphilic peptide being capable of undergoing ionic or physical crosslinking with the biocompatible polymer to form a porous film.

3. The preparation of claim 2, further comprising a buffer comprising an effective amount of an ionic salt to induce the purified amphiphilic peptide to form a predetermined secondary structure, wherein the purified amphiphilic peptide and the buffer are in the form of the predetermined secondary structure.

4. The preparation of claim 3, formulated as film comprising a hydrogel in the form of at least one of a cryogel, a dehydrated hydrogel, and a hydrated hydrogel.

5. The preparation of claim 2, comprising between about 0.15% by weight to about 10% by weight of the purified amphiphilic peptide.

6.-7. (canceled)

8. The preparation of claim 2, wherein the hydrophobic amino acid residues are independently selected from glycine, alanine, valine, leucine, isoleucine, proline, phenylalanine, methionine, threonine, tryptophan, and combinations thereof.

9. (canceled)

10. The preparation of claim 2, wherein the charged amino acid residues are independently selected from arginine, lysine, histidine, and combinations thereof.

11.-14. (canceled)

15. The preparation of claim 1, wherein the at least one functional group of the purified amphiphilic peptide comprises an amine, carboxyl, thiol, succinimidyl ester, maleimide, alkene, transcyclooctene, alkyne, ring-strained alkyne, dibenzylcyclooctyne, tetrazine, azide, or combinations thereof.

16.-20. (canceled)

21. The preparation of claim 2, wherein the folding group has a sequence comprising Y[XY]_{N}[T][YX]_{M}Y, where X is 1-3 charged amino acids, Y is 1-3 hydrophobic amino acids, T is 2-8 turn sequence amino acids, and N and M are each independently between 2 and 10.

22. The preparation of claim 2, wherein the turn sequence has 2-8 amino acid residues independently selected from a D-proline, an L-proline, aspartic acid, threonine, and asparagine.

23.-36. (canceled)

37. The preparation of claim 2, wherein the purified amphiphilic peptide is anionic or cationic, the cationic peptide having a net charge of from +2 to +11.

38.-57. (canceled)

58. The preparation of claim 2, comprising from about 0.5% by weight to about 10% by weight of the biocompatible polymer.

59. The preparation of claim 2, wherein the biocompatible polymer has a molecular weight of less than about 50 kDa.

60. The preparation of claim 2, wherein the biocompatible polymer is at least partially biodegradable, non-biodegradable, or combinations thereof.

61. The preparation of claim 1, wherein the at least two functional groups of the biocompatible polymer are independently selected from an amine, carboxyl, hydroxyl, thiol, succinimidyl ester, alkene, transcyclooctene, alkyne, ring-strained alkyne, dibenzylcyclooctyne, tetrazine, azide, maleimide, and combinations thereof.

62. (canceled)

63. The preparation of claim 2, wherein the biocompatible polymer is selected from a polyethylene glycol (PEG), derivative thereof or peptide conjugate thereof, polyethylene glycol-poly(lactide-co-glycolide) copolymer (PEG-PLGA), poly(ethylene glycol)-co-poly(glycolic acid) copolymer (PEG-co-PGA), polyvinyl alcohol (PVA), derivative thereof or peptide conjugate thereof, poly(2-hydroxyethyl methacrylate) (PHEMA), poly(N-isopropylacrylamide) (PNIPAAm), poly(acrylic acid) (PAAc), polyurethane, poloxamer, pluronics, polyurethane, polysaccharide, cellulose, carboxymethylcellulose, dextran, oxidized dextran, alginate, oxidized alginate, hyaluronic acid, chitosan, gelatin, elastin, collagen, carob gum, pullulan, and combinations thereof.

64. (canceled)

65. The preparation of claim 1, wherein the at least one chemical crosslinker molecule comprises glutaraldehyde, genipin, or a photo-crosslinker.

66. The preparation of claim 65, comprising from about 0.1% by volume to about 2% by volume of the glutaraldehyde.

67.-69. (canceled)

70. The preparation of claim 1, wherein the coupling chemical agent comprises an amide bond forming agent, a carbodiimide activation agent, a click chemistry agent, a copper-free click chemistry agent, a Michael-type addition agent, a Schiff base reaction agent, or combinations thereof.

71.-75. (canceled)

76. The preparation of claim 2, formulated as a film having a thickness of from about 0.1 mm to about 100 mm when hydrated and a thickness of from about 5 μm to 1000 μm when dehydrated.

77. (canceled)

78. The preparation of claim 2, wherein the film is at least partially nanoporous, microporous, macroporous, or combinations thereof.

79. The preparation of claim 78, formulated as a film having at least 75% pores by volume, wherein at least some of the pores are interconnected.

80. (canceled)

81. The preparation of claim 2, formulated as a film that is at least partially cryogelated, lyophilized, or combinations thereof, effective to control porosity.

82. The preparation of claim 2, formulated as a film for topical, buccal, or parenteral administration.

83.-97. (canceled)

98. The preparation of claim 2, formulated as a film, wherein the film is thermally stable between −20° C. and 150° C.

99. The preparation of claim 98, wherein the film is sterilized by terminal and/or autoclave sterilization.

100.-119. (canceled)

120. A kit for producing a film, comprising:
a biocompatible polymer capable of undergoing covalent, ionic or physical crosslinking with a peptide group;
a purified amphiphilic peptide comprising a folding group having a plurality of charged amino acid residues and hydrophobic amino acid residues arranged in a substantially alternating pattern and a turn sequence, and at least one functional group available for crosslinking;
a buffer comprising an effective amount of an ionic salt to induce the purified amphiphilic peptide to form a predetermined secondary structure; and
instructions to form the film by crosslinking the purified amphiphilic peptide with the biocompatible polymer.

121. The kit of claim 120, further comprising instructions to induce the purified amphiphilic peptide to form the predetermined secondary structure by combining the purified amphiphilic peptide with the buffer.

122. (canceled)

123. A method of producing a film, comprising:
crosslinking a purified amphiphilic peptide and a biocompatible polymer,
the biocompatible polymer being capable of undergoing crosslinking with a peptide group, the purified amphiphilic peptide comprising a folding group having a plurality of charged amino acid residues and hydrophobic amino acid residues arranged in a substantially alternating pattern and a turn sequence, and at least one functional group available for crosslinking.

124.-127. (canceled)

128. The method of claim 123, comprising covalently, ionically, or physically crosslinking the purified amphiphilic peptide and the biocompatible polymer, wherein the biocompatible polymer comprises a functional group capable of undergoing covalent, ionic, or physical crosslinking with the peptide group.
